(19) United States
(12) Patent Application Publication
(10) Pub. No.: US 2010/0003753 A1
(43) Pub. Date: Jan. 7, 2010

(54) Multivalent rna nanoparticles for delivery of active agents to a cell

(75) Inventor: Peixuan Guo, Mason, OH (US)

Correspondence Address: SEED INTELLECTUAL PROPERTY LAW GROUP PLLC, 701 FIFTH AVE, SUITE 5400, SEATTLE, WA, 98104, US

(73) Assignee: PURDUE RESEARCH FOUNDATION, WEST LAFAYETTE, IN (US)

(21) Appl. No.: 11/989,590

(22) PCT Filed: Aug. 1, 2006
(86) PCT No.: PCT/US2006/029787

§ 371 Date: Aug. 7, 2009

Related U.S. Application Data
(60) Provisional application No. 60/704,261, filed on Aug. 1, 2005.

(51) Int. Cl. C12N 5/00; C07H 21/02

(52) U.S. Cl. 435/377; 536/24.1

(57) ABSTRACT

A polyvalent multimeric complex formed from a plurality of chimeric pRNA molecules, each carrying at least one biologically active moiety, detectable label, or other heterologous component.

This application claims the benefit of U.S. provisional application Ser. No. 60/704,261, filed Aug. 1, 2005, and is a continuation-in-part patent application of U.S. patent application Ser. No. 10/539,241, filed Jun. 16, 2005; U.S. patent application Ser. No. 10/539,241 is a U.S. National Stage patent application of International patent application No. PCT/US 2003/039950, filed 16 Dec. 2003 (published as WO2005/003293 on Jan. 13, 2005); which claims the benefit of U.S. provisional patent application Ser. No. 60/433,697, filed Dec. 16, 2002, and is a continuation-in-part patent application of U.S. patent application Ser. No. 10/373,612, filed Feb. 24, 2003, which is a continuation-in-part application of International patent application PCT/US01/26333, filed Aug. 23, 2001, which in turn claims the benefit of U.S. provisional patent application Ser. No. 60/227,393, filed Aug. 23, 2000, each of which patent applications is incorporated herein by reference in its entirety.

### STATEMENT OF GOVERNMENT RIGHTS

This invention was made with government support under grants from the National Institutes of Health (Grant Nos. GM59944, GM48159 and R01-EB003730), the Department of Defense (Breast Cancer Concept award BC024308 and Grant Nos. W81XWH-05-0158 and DAMD17-03-1-0589); and the National Science Foundation (Grant No. MCB-9723923). The government has certain rights in this invention.

### BACKGROUND

Nanotechnology has brought about an unprecedented variety of revolutionary approaches for the detection and therapy of diseases. Due to their small size, nanoparticles can readily interact with biomolecules either on the surface of or within cells. To take advantage of this, it is desirable to develop multifunctional engineered, targeted complexes capable of bypassing biological barriers to deliver multiple therapeutic agents directly to specific cells or tissues. Due to their easy access to many areas of the body, multivalent nanoparticles offer the possibility of a wealth of innovative tools with the potential to combine detection and therapy in ways previously unimaginable.

At different stages of cancer development, the abnormal and malfunctioning cells express a variety of cellular factors, signaling molecules, markers, receptors and other specific antigens. Earlier detection and treatment of cancer with unique signatures by multivalent therapeutic agents or detection sensors could greatly benefit patients and save lives. (Hood, et al., Science 2002; 296:2404-2407). However, developing the ability to detect stage-specific characteristics of cancer cells and thereby produce targeted imaging and multiple-drug delivery systems remains a challenge. It is difficult for particles larger than 50 nm to enter cells; the size limit for endocytosis is about 100 nm. Molecules smaller than 20 nm could move out of blood vessels during circulation and have a shorter retention time in the body.

Small interfering siRNA (Elbashir et al. (2001), Nature, 411, 494-498; McCaffrey et al. (2002), Nature, 418, 38-39; Li et al., Science 2002; 296:1319-1321; Ryther et al., Gene Ther 2005; 12:5-11; Brummelkamp et al., Science 2002; 296;550-553; Jacque et al., Nature 2002; 418:435-438; Carmichael, Nature 2002; 418:379-380; Soutschek et al., Nature 2004; 432(7014):173-178), ribozymes (Hoeprich et al., Gene Therapy 2003, 10(15), 1258-1267; Rossi, Adv Drug Deliv Rev 2000; 44:71-78) and anti-sense RNA (Coleman et al., Nature 1985; 315:601-603; Knecht et al., Science 1987; 236:1081-1086; Kumar et al. (1998), Microbiol Mol Biol Rev, 62, 1415-1434) all show significant potential in new molecular approaches to down-regulate specific gene expression in cancerous or viral-infected cells.

siRNA and ribozyme RNA have been extensively utilized for post-transcriptional gene silencing in a sequence-specific manner. The delivery of siRNA has been studied by various methods, including viral vector delivery (Devroe et al., Expert Opin Biol Ther 2004; 4:319-327) and lipid-encapsulated RNA injection (Morrissey et al., Nat Biotechnol 2005; 23:1002-1007). Recently, siRNAs joined to a cholesterol group have been reported to silence target gene expression in mice via intravenous injection. (Soutschek et al., Nature 2004; 432:173-178). siRNA was also successfully used for knocking down HIV-related gene expression (Novina et al. (2002), Nat Med, 8, 681-686; Akkina et al. (2003), Anticancer Res, 23, 1997-2005; Mariadason et al., Cancer Res 2003; 63:8791-8812).

A ribozyme is an RNA molecule capable of cleaving a target RNA molecule, or carrying out other catalytic and enzymatic functions. Structurally, it is single-stranded RNA characterized by two “arms” positioned either side of a small loop. The ribozyme base pairs to a region on the target RNA that is complementary to the nucleotide sequence of its two arms. The loop region serves as an active catalytic center that performs the cleaving function on the target RNA (FIG. 1).

The use of ribozymes for treatment and prevention of diseases in plants, humans and animals has the potential to revolutionize biotechnology. Hammerhead ribozymes have, for example, been used to cleave RNA in transgenic plants and animals. However, despite numerous publications reporting the results of investigations in test tubes, reports on the successful use of hammerhead ribozymes in living organisms are relatively few (Perriman et al., Proc. Natl. Acad. Sci. USA 92:6175-6179 (1995)). Although it is clear that hammerhead ribozymes can cleave specific viral RNA or mRNA in test tubes, the efficiency of cleavage in cells is dramatically reduced due to instability and misfolding of the ribozyme in cells.

A major cause for the instability of ribozymes in an intracellular environment is degradation of the ribozyme by exonuclease present in the cells (Cotton et al., EMBO J. 8:3861-3866 (1989)). Exonucleases are enzymes that nonspecifically trim RNA from both ends. One method that has been used to block the intracellular degradation of ribozymes is to protect the ribozyme by connecting it at one end to a vector RNA, such as tRNA (Vaish et al., Nucl. Acids Res. 26:5237-5242 (1998)). However, due to refolding of the resulting chimera RNA, the ribozyme varied in efficiency compared to the unprotected ribozyme (Bertrand et al., RNA 3:75-88 (1997)). Tethering of a ribozyme to both ends of a tRNA has also been reported, but folding and/or activity was compromised (Vaish et al., Nucl. Acids Res. 26:5237-5242 (1998)).

The potential to treat disease by using libozymes to cleave RNA involved in cancer and pathogen infection is tremendous. The availability of a stabilized ribozyme that is resistant to degradation and is correctly folded such that it remains active in an intracellular environment would pave the way for the development of many important medical therapies.

The successful application of siRNAs and ribozymes for the treatment of cancer and other diseases and conditions requires overcoming the following obstacles: 1) difficulty entering the cell due to the size limit for membrane penetration; 2) degradation by exonucleases within the cell; 3) trafficking into the appropriate cell compartment; 4) correct folding of the ribozymes or siRNA in the cell if fused to a carrier; and 5) the recognition of target cells. At this time, the development of a safe, efficient, specific and nonpathogenic nanoparticle for the delivery of multiple therapeutic RNAs is highly desirable.

### SUMMARY OF THE INVENTION

The present invention provides a chimeric pRNA that includes a paired double-stranded helical domain, an intermolecular interaction domain, and a heterologous component. The heterologous component confers a desired property on the chimeric pRNA, and may take the form of a biologically active moiety (e.g., a therapeutic agent), a detectable label, a stabilizing agent, and the like.

In one embodiment, the chimeric pRNA is a circularly permuted chimeric pRNA molecule. The pRNA chimera is formed from a circularly permuted pRNA region, and a spacer region that includes the heterologous component. The heterologous component is not limited to any chemical structure but is preferably a biologically active moiety, more preferably an RNA, such as a ribozyme, siRNA (small, interfering RNA), an RNA aptamer or an antisense RNA. The spacer region is covalently linked at its 5′ and 3′ ends to the pRNA region. Optionally, the spacer region includes first and second nucleotide strings interposed between the biologically active moiety and the pRNA region.

In another embodiment, the pRNA chimera of the invention includes, as its heterologous component, an siRNA, such that the paired double-stranded helical region includes or is formed from the siRNA. The siRNA is effective to silence a gene expressed in cell, for example a cancer cell or a cell infected by a virus. Examples of genes that can be silenced include survivin and various viral genes.

In another embodiment of the pRNA chimera of the invention, the heterologous component is linked, covalently or noncovalently, to the pRNA at or near the 5′ or 3′ end of the pRNA. Preferred heterologous components for linking at or near the 5′ or 3′ ends of the pRNA include targeting moieties, such as folate, and detectable labels, such as biotin, fluorescent labels, and radiolabels. In a particularly preferred embodiment, the pRNA has a 5′ overhanging end, and the heterologous component is linked to the 5′ overhanging end.

In yet another embodiment of the pRNA chimera of the invention, the heterologous component includes an oligonucleotide annealed to (via base-pairing/hybridization) the 5′ or 3′ end of the pRNA. Preferably, the oligonucleotide is a DNA oligonucleotide although it may be an RNA oligonucleotide. In a particularly preferred embodiment, the pRNA has a 5′ or a 3′ overhanging end, and the oligonucleotide anneals to the overhanging end, preferably the 3′ overhanging end. Optionally, the oligonucleotide includes a detectable label, such as a biotin or a radiolabel.

The chimeric pRNA of the invention can be monomeric or multimeric. When multimeric, the pRNA is preferably a dimer, a trimer or a hexamer, allowing the multimeric complex to be polyvalent. In a polyvalent multimeric complex, the multiple heterologous components may be the same or different. The multimeric complex may advantageously contain one or more biologically active moieties that facilitate specific targeting to deliver one or more therapeutic agents carried by other constituent chimeric pRNAs, such as biological moieties involved in cell surface binding, membrane diffusion or endocytosis. For example, the SELEX approach has been commonly used to screen for RNA aptamers that bind cell surface markers (Ellington et al., Nature 346, 818-822 (1990); Tuerk et al., Science 249, 505-510 (1990)). Such RNA aptamers can be attached to one of more subunits of the pRNA dimer, trimer or hexamer for specific cell recognition during delivery of the therapeutic agent. Other heterologous components that can be included in the multimeric complex include those involved in intracellular targeting and release of the therapeutic agent, labeling components, stabilizing agents such as complementary oligonucleotides, and the like.

The pRNA region has a compact stable secondary structure characteristic of bacteriophage pRNA sequences. Thus, in one embodiment of the pRNA chimera, the pRNA region includes a pRNA of a bacteriophage selected from the group consisting of φ29, SF5′, B103, PZA, M2, NF and GA1. The pRNA may be circularly permuted or not circularly permuted. In another embodiment of the pRNA chimera, the pRNA region includes:
(i) in the 5′ to 3′ direction beginning at the covalent linkage of the pRNA with the 3′ end of the spacer region
   a first loop;
   a second loop; and
   a lower stem-loop structure comprising a bulge,
a first stem section and a third loop;
(ii) a second stem section interposed between the spacer region and the stem-loop structure;
(iii) a third stem section interposed between the stem-loop structure and the first loop;
(iv) a fourth stem section interposed between the first loop and the second loop; and
(v) an opening defining 5′ and 3′ ends of the pRNA chimera, positioned anywhere within the pRNA region.

The invention also provides a method for making a pRNA chimera of the invention. In one embodiment, DNA encoding a pRNA chimera containing a pRNA region and a spacer region that includes a biologically active RNA is transcribed in vitro to yield the pRNA chimera. Optionally, the DNA encoding the pRNA chimera is generated using polymerase chain reaction on a DNA template, or the DNA is generated by cloning the DNA into a plasmid and replicating the plasmid. In another embodiment, the pRNA is chemically synthesized using smaller RNA fragments (modular components).

The invention further provides a method for determining whether an RNA molecule interacts with a test molecule. A pRNA chimera that includes the RNA molecule of interest is immobilized on a substrate, then contacted with test molecule. Whether or not the test molecule interacts with the RNA of interest, such as by binding the RNA of interest, is then detected.

The invention also provides a DNA molecule that includes a nucleotide sequence that encodes a pRNA chimera containing a pRNA region and a spacer region that includes a biologically active RNA.

Also provided by the invention is a method for delivering a biologically active RNA to a cell, preferably a plant cell or an animal cell, such as human cell. In one embodiment, a DNA molecule having a nucleotide sequence that operably encodes a pRNA chimera of the invention is introduced into the cell and transcribed to yield a biologically active RNA. In another embodiment, the pRNA chimera is directly transfected into the cell. Alternatively, the chimeric pRNA complex can be delivered to the cell via endocytosis by the incorporation of RNA aptamers that specifically bind to cell surface markers (Ellington et al., Nature 346, 818-822 (1990); Tuerk et al., Science 249, 505-510 (1990)).

### BRIEF DESCRIPTION OF THE FIGURES

FIG. 1 is a schematic depiction of target RNA cleavage by a representative ribozyme.

FIG. 2 depicts the nucleotide sequence (SEQ ID NO:1) and secondary structure of wild-type φ29 (phi29) pRNA (SEQ ID NO: 27) indicating (a) the location and nomenclature of the loops and bulges (Zhang et al., RNA 3:315-323 (1997)); and (b) the procapsid binding domain and the DNA packaging domain; the right and left-hand loops, the head loop, the U⁷²U⁷³U⁷⁴ bulge, and the C¹⁸C¹⁹A²⁰ bulge are in boxes; the DNA-packaging domain (5′/3′ ends) and the procapsid binding domain (the larger area) are shaded; the curved line points to the two interacting loops; note that the three base UAA 3′ overhang shown in (a) is absent in this diagram.

FIG. 3 depicts that nucleotide sequences of several pRNAs prior to circular permutation: (a) bacteriophage SF5′ (SEQ ID NOS:11 and 28), (b) bacteriophage B103 (SEQ ID NOS:12 and 29), (c) bacteriophages φ29 and PZA (SEQ ID NOS:13 and 30)_, (d) bacteriophage M2 and NF (SEQ ID NOS:14 and 31), and (e) bacteriophage GA1 (SEQ ID NOS:15 and 32) (Chen et al., RNA 5:805-818 (1999); and (f) aptRNA (SEQ ID NOS:16 and 33).

FIG. 4 is a schematic depiction of various structural features of a pRNA chimera of the invention: (a) a whole pRNA chimera; (b) a spacer region component; (c) a pRNA region component.

FIG. 5 is a schematic depiction of (a) the design of one embodiment of the pRNA chimera of the invention; and (b) exemplary circularly permuted pRNA (cpRNA) molecules showing various locations for the circle openings.

FIG. 6 depicts (a) a possible mechanism of pRNA-ribozyme cleavage activity; and (b) the structural arrangement of the chimeric pRNA/ribozyme complex.

FIG. 7 depicts (a) the sequence and predicted secondary structure of wild-type pRNA (SEQ ID NO:1); (b) the secondary structure of a pRNA dimer (SEQ ID NO:26) (Trottier et al., RNA 6:1257-66 (2000)); (c) a three dimensional computer model of a pRNA dimer (Hoeprich and Guo, J Biol Chem 277:20794-803 (2002)); wherein the lines between residues of the monomer subunits of the dimer in (b) show the bases of the left and right hand loops interact intermolecularly via hand-in-hand interaction (Guo et al., Mol Cell 2:149-55 (1998); Zhang et al., Mol Cell 2:141-47 (1998)); (d) and (e) diagrams depicting the formation of a pRNA hexameric ring by upper and lower loop sequence interaction.

FIG. 8 depicts various embodiments of a pRNA dimer, trimer and hexamer as a polyvalent gene delivery vector.

FIG. 9 depicts the use of circularly permuted pRNA in the SELEX method to identify RNA aptamers that bind to a pre-identified substrate. NNN . . . N(25-100) . . . NNN, random sequence of template; template (SEQ ID NO: 36), template primer; primer 1 (SEQ ID NO: 35), 3′ end primer; primer 2 (SEQ ID NO: 34), 5′ end primer.

FIG. 10 presents the impact of various extensions (SEQ ID NOs:19-22) of the 3′ end of the pRNA on viral activity as measured by plaque forming units.

FIG. 11 depicts the design and production of circularly permutated pRNAs. The DNA template in (a) (SEQ ID NO:2) uses a short (AAA) sequence to join the native 5′/3′ ends, while the template in (b) (SEQ ID NO:38) uses a longer sequence (SEQ ID NO:8) to join the native 5′/3′ ends. New openings of the cpRNA are indicated by the wedges pointing to places in the transcript sequences (SEQ ID NOS: 37 and 39). (See Zhang et al., RNA 3:315-323 (1997)).

FIG. 12 depicts an RNA chimera (residues 1-167 of SEQ ID NO:3) bound to a portion of the U7snRNA substrate (SEQ ID NO:4).

FIG. 13 depicts in vitro cleavage of substrates by chimeric ribozyme carried by pRNA. (a) Schematic showing secondary structure of RzU7, the U7 snRNA targeting ribozyme (SEQ ID NO:10), base-pairing with its substrate (SEQ ID NO:4). (b) Denaturing urea gel showing cleavage of the substrate U7snRNA into its expected 69 mer and 25 mer cleavage products by both the ribozyme RzU7 and the chimera ribozyme pRNA-RzU7.

FIG. 14 depicts a denaturing urea gel evidencing successful cleavage of the substrate HBV-polyA into its expected 70 mer and 67 mer cleavage products.

FIG. 15 depicts the design and construction of plasmid encoding the self-process ribozyme targeting at the HBV polyA signal. (a) shows the design of plasmid encoding ribozyme pRNA-RzA. (b) shows the processed chimeric ribozyme after transcription and cis-cleavage. (c) shows the secondary structure of the hammerhead ribozyme (RzA) (SEQ ID NO:23) base paired to the HBV polyA target sequence (SEQ ID NO:24). An indicated change from “G” to “A” generated an inactive enzyme as negative control. (d) shows secondary structure of the ribozyme pRNA-RzA (SEQ ID NO:25) base paired to the HBV polyA substrate (SEQ ID NO:24).

FIG. 16 depicts the effect of ribozymes on HBV RNA levels in HepG2 cells.

FIG. 17 depicts an anti-12-LOX ribozyme (SEQ ID NO:5) bound to substrate RNA (SEQ ID NO:6).

FIG. 18 depicts formation of pRNA dimer, trimer and hexamer via the interaction of the right (uppercase letter) and left (lower case letter) hand loop. The same letters in upper and lower cases, e.g. A and a′, indicate complementary sequences, while different letters, e. g. A and b′, indicate non-complementary loops.

FIG. 19 depicts secondary structure, domain and location of pRNA on phi29 viral particle: (a) secondary structure of pRNA A-b′ (SEQ ID NO: 40). The intermolecular binding domain (shaded area) and the reactive DNA translocation domain are marked with bold lines. The four bases in the right and left loops, which are responsible for inter-RNA interactions, are boxed; (b) Power Rangers depict pRNA hexamer by hand-in-hand interaction; (c) phi29 DNA-packaging motor with pRNA hexamer formed by pRNA A-b′ and B-a′. The surrounding pentagon stands for the fivefold symmetrical capsid vertex, viewed as end-on with the virion at side-view. The central region of pRNA binds to the connector and the 5′/3′ paired region extends outward (Chen et al., RNA, 5:805-818 (1999)).

FIG. 20 depicts (a) two; (b) three and (c) six interlocking pRNAs.

FIG. 21 depicts a representative chimeric pRNA design. I. The chimeric pRNA harboring a ribozyme hybridized to a target. II. The secondary structure of a pRNA monomer (SEQ ID NO: 1). III. The secondary structure of a chimeric pRNA (SEQ ID NO: 41) harboring a ribozyme (SEQ ID NO: 44) targeting an HIV tat/rev substrate. IV. The secondary structure of a chimeric pRNA harboring an adenovirus knob-binding aptamer (SEQ ID NO: 43). V. The secondary structure of a chimeric pRNA harboring a CD4-binding RNA aptamer (SEQ ID NO: 42). The three pRNAs combine to form a trimer.

FIG. 22 shows a schematic diagram of the engineering and fabrication of RNA nanoparticles. (I) Size and three-dimensional computer model of phi29 pRNA vector. (II) The fabricated trimer.

FIG. 23 shows six different configurations of trimer designs containing aptamer, siRNA, ligand, and fluorescence.

FIG. 24 shows confocal microscopy showing the specific and simultaneous delivery of three components to CD4-overexpressing cells. I. Assay for the binding of pRNA trimer containing pRNA(A-b′)/aptamer(CD4), pRNA(B-e′)-FITC, and pRNA(E-a′)-Rhodamine to CD4^{hi} T cells (A-D of left column, and I-L of right column) and CD4^{neg} T cells (E-H of middle column). A, E, and I were imaged with an FITC filter; while B, F, and J were viewed with a Rhodamine filter; C, G, and K are overlays; and D, H and L are DIC images. The right column represents a close-up view of CD4^{hi} cells. Arrows point to the complexes that had entered the cell. II. Section of confocal microscopy images to differentiate between binding (M) and cell entry (arrows in N and O) as well as negative control (P). Binding of FITC-labeled pRNA trimer containing CD4-binding aptamer to lymphocytes was shown as a circle, and entry was shown as a green spot inside cell (arrow in O). The red color in N is a positive entry control of transferrin labeled with Texas red.

FIG. 25 shows native polyacrylamide gel (A), AFM imaging (B) and sucrose gradient sedimentation (C) to detect the formation of the fabricated pRNA trimers. (A) Native PAGE gel showing pRNA monomer and trimer of pRNA chimeras exhibiting different migration rates. (B). AFM images of pRNA monomer and trimer with low and high magnification. The pRNA monomers folded into a checkmark shape, and the trimer exhibited a triangular shape. The color within each image reflects the thickness and height of the molecule. Brighter (or whiter) color indicates a thicker or taller molecule; darker color indicates a thinner molecule. (C.) Separation of pRNA monomers and trimers by 5-20% sucrose gradient sedimentation. All particles were loaded onto the top of the gradient and separated by ultracentrifugation. Sedimentation is from right to left.

FIG. 26 shows a GFP knockdown assay of different chimeric siRNA constructs. Two days after cells being transfected with various chimeric pRNA/siRNA constructs, GFP expression was observed by fluorescence microscope.

FIG. 27 shows a functional assay of siRNA(CD4) co-delivered via the fabricated pRNA trimer harboring aptamer(CD4), siRNA(CD4), and pRNA/FITC. (I) Positive control showing the co-delivery of pRNA(A-b′)/aptamer(CD4), pRNA(B-e′)-FITC, and pRNA(E-a′)-Rhodamine in trimers (FIG. 23A). Incubation, not transfection, of CD4^{hi} T cells with the trimer resulted in 95% of the cells taking up both FITC and Rhodamine fluorescent RNAs mediated by aptamer(CD4), as assessed by flow cytometry. (II) Incubation, not transfection, of CD4^{hi} T cells with pRNA trimer containing pRNA(A-b′)/aptamer(CD4), pRNA(B-e′)-FITC, and pRNA(E-a′)-siRNA(CD4) (FIG. 1-III-C) resulted in 85% of the cells taking up FITC. The CD4^{hi} T cells taking up the pRNA trimer were divided into FITC-positive and FITC-negative cells, and CD4 levels were determined by surface staining with a PE-labeled antibody. In the FITC-positive cells, the CD4 level was reduced to 17.8%, in comparison with 42.56% for the FITC-negative cells, demonstrating the delivery and function of the siRNA(CD4) in the trimers. (III) Negative controls showing that incubation of CD4^{hi} T cells with pRNA dimers or trimers harboring aptamer(CD4) but not siRNA(CD4) did not make difference in CD4 level (all around 90%) and cell viability (all around 70%). The control dimers or trimers contained pRNA/siRNA(BIM) instead of pRNA/siRNA(CD4).

FIG. 28 shows processing of chimeric pRNA/siRNA complex into siRNA by cell lysates (C) or purified Dicer (D). The pRNA/siRNA monomer (lane b-e), phi29 pRNA vector (lane f-I, & m-o), or the trimeric chimera (j-l) were labeled with ³²P at the 5′-end and incubated with cell lysates (C) or Dicer (D) and analyzed by denaturing gel. (E) and (F) shows the inhibition of pro-apoptosis factor BIM with specific pRNA/siRNAs protected lymphocytes from cytokine withdrawal and induced apoptosis. D1 (T cells) is cytokine-dependent lymphocyte cell lines that express BIM, and exhibit apoptosis in the absence of cytokines. Introduction of pRNA/siRNA(BIM) into D1 cells (E) resulted in protection from IL-3 withdrawal-induced cell death. Protein levels of BIM (F) were assayed by Western blot.

FIG. 29 shows animal trials for cancer therapy using the fabricated RNA nanoparticles. (A) Injection without the pRNA/siRNA chimera (No RNA); (B) Treatment with RNA chimera containing folate-pRNA and siRNA(survivin); (C) Treatment with RNA chimera containing folate-pRNA and siRNA(survivin) with mutations in the siRNA sequence; (D) Treatment with pRNA-siRNA chimera that does not contain a folate at its 5′ end.

FIG. 30 shows a sketch of sequence and structure of pRNA chimeras. (A) phi29 pRNA sequence and secondary structure. The double-stranded helical domain on the 5′/3′ ends is framed, and the domain for dimer formation is shaded. The curved line points to the two interacting loops. (B) Three-dimensional structure of pRNA dimer. (C) Native polyacrylamide gel showing monomer and dimer of the pRNA chimeras exhibiting different migration rates. Below the gel are cryo-AFM images of phi29 pRNA monomer and dimer. The colors reflect the thickness and height of the molecule; the brighter the color, the thicker or taller the molecule. (D) Design of chimeric pRNA dimers harboring foreign moieties (see Nomenclature of RNA Subunits, under Results).

FIG. 31 shows processing of chimeric pRNA/siRNA complex by Dicer. The structures of pRNA/siRNA and pRNA vector are shown in (A) and (B). Processing of pRNA/siRNA into 22-bp siRNAs by recombinant Dicer. The chimeric pRNA/siRNA with 5′-end 32P labeling was incubated with purified recombinant Dicer for 30 min and 2 hr, respectively, and then separated on a denaturing PAGE/urea gel. A radiolabeled 22-nucleotide RNA was used as a molecular weight marker.

FIG. 32 shows a functional assay of chimeric pRNA/siRNA(GFP) by transfection. (A-C) Fluorescence microscopy images showing the silencing of GFP gene by transfection. (A) Dose-dependent silencing of GFP gene by chimeric pRNA/siRNA(GFP) (left column). A mutant pRNA/siRNA (right column) served as negative control. (B) GFP expression of cells transfected with various RNAs: (a) no RNA; (b) synthesized double-stranded siRNA(GFP); (c) double-stranded siRNA(LacZ) control; (d) pRNA/siRNA(GFP); (e) pRNA/siRNA(mutant); (f) pRNA vector alone. (C) Comparison of the performance of (a) chimeric pRNA/siRNA(GFP) and (b) conventional double-stranded siRNA(GFP) at the same molar concentration; (c) control with no siRNA treatment. (D) Northern blot to examine the effect of chimeric pRNA/siRNA(GFP) on GFP mRNA level after transfection. Lanes 1 and 2 show the effects of two different constructs of pRNA/siRNA(GFP); lane 3, double-stranded siRNA; lane 4, cells without RNA treatment rRNA was used as loading control.

FIG. 33 shows a functional assay of chimeric pRNA/siRNA targeting luciferase by transfection. (A) Dual reporter luciferase assay showing the specific knockdown of firefly luciferase or Renilla luciferase expression by pRNA/siRNA(firefly) or pRNA/siRNA(Renilla), respectively, in a dose-dependent manner. (B) Comparison of the activities of conventional hairpin siRNA(luciferase) and pRNA/siRNA(luciferase). pRNA/siRNA(mutant) with mutations in siRNA sequences was included as a nonspecific control.

FIG. 34 shows apoptosis and cell death induced by transfection of chimeric pRNA harboring siRNA targeting survivin. (I) Breast cancer MCF-7 cells were transfected with pRNA/siRNA(survivin) and apoptosis was monitored by PI-annexin V double-labeling followed by flow cytometry. Cells in the bottom right quadrant represent apoptotic cells. (II) Breast cancer cells (MDA-231) and prostate cancer cells (PC-3) were transfected with 20 pmol of pRNA/siRNA(survivin) in 24-well plates and images were taken 24 hr after transfection. The mutant pRNA/siRNA was transfected in parallel as a negative control.

FIG. 35 shows a functional assay of pRNA/siRNA chimera targeting proapoptotic factor BAD. (A) pRNA/siRNA(BAD) and controlsiRNAs (10 nM) were introduced into pro-B cells by electroporation, combined with a transfection reagent on day 1. Cells were washed to remove IL-3 on day 2 and assayed for viability on day 3. (B) BAD protein levels were compared in cells transfected with chimeric siRNA(BAD) or two mutant controls containing different mutations within siRNA sequences. Control cells were treated with pRNA alone. Cell lysates were prepared (Khaled et al., 2001) on day 3 and proteins were separated by 12% SDS-PAGE followed by Western blot with BAD antibody (Cell Signaling Technology, Beverly, Mass.). Numbers below the panel indicate the remaining BAD level, expressed as a percentage compared with control cells. (C) Pro-B cells transfected with pRNA/siRNA(survivin) or mutant were grown in complete medium containing IL-3 or deprived of IL-3. The impact of RNA on cell morphology was observed by microscopy.

FIG. 36 shows specific delivery of chimeric pRNA/siRNA by CD4 receptor. (I) Green circles corresponding to the binding of FITClabeled pRNA dimer containing CD4-binding aptamer to lymphocytes were shown by confocal microscopy (a) and the entry of RNA was shown as a green spot inside the cell (d). Texas Red-labeled transferrin was used as a positive control of internalization (b). No binding was observed in the control cell line without CD4 expression (c). Inset: Differential interference contrast (DIC) picture of the cells used for staining. (II) Incubation of RNA dimer led to the specific suppression of cell viability, as measured by trypan blue exclusion assay. Three different cell lines with no, low, or high CD4 expression level were incubated with the pRNA/siRNA(survivin)-pRNA/aptamer(CD4) complex in the presence or absence of IL-7.

FIG. 37 shows a toxicity assay of chimeric pRNA targeting survivin. HeLaT4 cells were seeded in 24-well plates so that they will be 30-50% confluent at the time of incubation. Twenty-four hours after seeding, the indicated amount of RNA was added into each well. Cells were further incubated in incubator for 24, 48, and 72 hr and the number of viable cells was counted by hemacytometer. The relative survival rates displayed were obtained by dividing the number of surviving cells in each treatment by the number of surviving cells without treatment with RNA.

FIG. 38 shows specific delivery of chimeric pRNA/siRNA by folate-pRNA. (A) Flow cytometry analyses of the binding of FITC-labeled folate-pRNA to KB cells. Left: Cells were incubated with folate-pRNA labeled with FITC. Middle: Cells were preincubated with free folate, which served as a blocking agent to compete with folate-pRNA for binding to the receptor. Right: Binding was also tested using folate-free pRNA labeled with FITC as a negative control. The percentages of FITC-positive cells are shown in the top right quadrants. (B) Specific binding of folate-pRNA dimer to KB cells. After incubation of cells with the [3H]folate-pRNA dimer in the presence (middle column) or absence (left column) of free folate, cells were isolated and subjected to scintillation counting. The right-hand column represent 3H-labeled dimer without folate labeling as a negative control. (C) In a knockdown assay by incubation, folate-chimeric dimer complex containing pRNA(B-a′)/folate and pRNA(A-b′)/siRNA(firefly) was incubated with IB cells for 3 hr to allow the binding and entry of RNA. The luciferase level was measured the next day in the dual reporter system. The control dimer was identical to the folate dimer except for its lack of folate labeling.

FIG. 39 shows the potential use of pRNA hexamers as polyvalent gene delivery vectors. Six copies of pRNA have been found to form a hexameric ring to drive the DNA-packaging motor of bacterial virus phi29. There would therefore be six positions available to carry foreign moieties for targeting, therapy, and detection.

FIG. 40 shows structure, purification and characterization of folate-AMP. (A). The binding of folate-AMP to the folate receptor shown by a competition assay. KB cells were left without treatment (a) or incubated with folate-FITC (c). Also shows the binding of FITC without folate labeling (b). The numbers shown in the upper right quadrants represent the percentage of fluorescence-positive cells. The binding of folate-FITC to KB cells was blocked with free folate (d) or folate-AMP (e). (B). Structure of folate-AMP. (C). Purification of folate-AMP by reverse phase HPLC under the following conditions: Column: Delta Pak C18 7.8×300 mm, pre-equilibrated with 20 mM phosphate, pH 7.0; flow rate at 6 mL/min. After sample loading, the eluting solvent was changed manually according to the following orders: from 100% 20 mM phosphate to 100% water at 8 min, to 10% MeOH/90% water at 19 min, and finally to 15% MeOH/90% water at 20 min. The 22-31 min folate-AMP fraction was collected and lyophilized.

FIG. 41 shows 5′ labeling of pRNA by folate and binding of folate-pRNA to KB cells. (A) Folate-AMP was included in the in vitro transcription together with non-modified NTPs. Trace amounts of [α³²P] ATP were included to indicate the position of RNA. Only one major band was detected on the PAGE/Urea gel when folate-AMP was skipped. About 50%-60% of the RNA shifted to the upper position when 2 mM and 4 mM folate-AMP were utilized, respectively. (B) Binding of [³H]-labeled folate-RNA to KB cells.

FIG. 42 shows a sketch of the sequence and structure of pRNA chimeras. (A) Phi29 pRNA sequence and secondary structure. The right- and left-hand loops are circled, the double helical domain is framed, and the intermolecular interacting domain is shaded. The curved line points to the two interacting loops. (B) 3D structure of pRNA monomer. (C) Illustration of the construction of folate-labeled pRNA (7-106) with truncated sequences. (D) Folate-labeled chimeric pRNA dimers harboring siRNA against survivin (E) The high-efficiency formation of pRNA dimer was demonstrated by native-PAGE. (F) Specific binding of folate-pRNA dimer to KB cells. Cells were incubated with RNA dimer composed of folate-pRNA (A-b′) and [³H]-pRNA (B-a′) in the presence (center column) or absence (left column) of free folate. The right column is the [³H]-dimer without folate labeling as a negative control.

FIG. 43 shows specific knockdown of gene expression by transfection of pRNA/siRNA. Dual luciferase assay showing the specific silencing of the gene for firefly luciferase (left) and renilla luciferase (right) by pRNA/siRNA(Firefly) and pRNA/siRNA(Renilla), respectively, by transient transfection.

FIG. 44 shows processing of chimeric pRNA/siRNA complex by recombinant Dicer into 22-bp siRNA. The chimeric pRNA/siRNA (lane a-c) or pRNA vector (lane e-f) with 5′-end [γ³²P] labeling was incubated with purified recombinant Dicer for 30 min and 2 h, respectively, and then separated on denaturant PAGE/Urea gel. 22 nt RNA was used as marker. The processing of dimeric RNA (lane a & d) was also examined.

FIG. 45 shows a sketch of sequence and structure of pRNA chimera. (A) Sketch of chimeric ribozyme harbored in pRNA vector. (B) pRNA/RZ(Sur) sequence and secondary structure. The highlighted G75 is deleted in pRNA/RZ(mut1). In pRNA/RZ(mut2), G75 is deleted and G58 is replaced with an “A”. (C) Sequence of pRNA/RZ(mut 3).

FIG. 46 shows (A) Cell morphology of MCF-7 after treatment of pRNA/RZ(Sur) chimera. MCF-7 were transfected with pRNA/RZ(Sur), pRNA/RZ(mut3) at high and low dose. One day after transfection, images were taken using an inverse microscope. (B). PI/annexin V double-staining to differentiate apoptosis from necrosis. Breast cancer MCF-7 cells were transfected with pRNA/RZ(sur) and apoptosis was monitored using PI/annexin V double-labeling followed by flow cytometry. Three parallel experiments were performed and the percentage of apoptotic cells was shown with standard deviation.

FIG. 47 shows the effects of chimeric survivin ribozyme on breast cancer cells. (A). MDA-MB-231 and (B). MDA-MB-453 cells were transfected with indicated amount of RNA and cell viability was measured in the next day.

FIG. 48 shows the effects of chimeric survivin ribozyme on cervix cancer cells. The human cervix cancer Hela T4 cells were transfected with pRNA/RZ(sur) with indicated amount and cell viability was measured by 48 assay in the next day.

FIG. 49 shows he effects of chimeric survivin ribozyme on nasopharyngeal cancer cells. The human nasopharyngeal cancer KB cells were transfected with pRNA/RZ(sur) with indicated amount and cell viability was measured by MTT assay in the next day.

FIG. 50 shows the effects of chimeric survivin ribozyme on prostate cancer cells. The human prostate cancer LNCaP cells were transfected with pRNA/RZ(sur) and cell viability was measured by MTT assay in the next day.

FIG. 51 shows a comparison of mRNA levels of different pRNA chimera treated samples revealed by real-time PCR. Gene expression level was compared to the level of gene expression found in non-transfected sample, arbitrarily assigned the value 1. Bars represent the fold number in gene expression over the expression level in the non-transfected samples. For samples transfected with pRNA/RZ(sur), the mRNA level decreased to 16% of that of the non-transfected cells, 24% of pRNA/RRZ(mut3) treated cells.

FIG. 52 shows a comparison of survivin protein level by Western blot after T47D cells were transfected with chimeric pRNA/RZ(sur) or mutant chimeric ribozyme.

FIG. 53 shows (A). Dimer and trimer formation of pRNA/RZ(sur) as shown in 8% native PAGE. (B). The cleavage of partial sequence of survivin mRNA by pRNA/RZ(sur). The substrate RNA is partial sequence of mRNA of human survivin labeled with [³²P]. (C). Deliverable dimer and trimeric complex.

FIG. 54 shows (A) Table 4. DNA oligonucleotides used for the production of pRNA chimera; and (b) Table 5. Functional assay of chimeric pRNA/ribozyme targeting survivin.

FIG. 55 shows a sph1-pRNA chimera, which has a 3′ RNA extension (i.e., an overhanging, unpaired 3′ end) and a complementary DNA oligonucleotide tag annealed thereto.

FIG. 56 shows gel electrophoresis of the results of a pRNA toxicity study of various sph-1pRNA constructs.

FIG. 57 shows the results of pRNA toxicity studies of (A) sph1-pRNA with different DNA tails annealed to the 3′ end; (B) sph1-pRNA/siRNA(luciferase); (C) sph1-pRNA/siRNA(survivin); and (D) sph1-pRNA/siRNA(GFP), with different DNA tails annealed to the 3′ ends.

FIG. 58 shows inhibition of GFP expression in Drosophila S2 cell by various chimeric pRNA/siRNA. The sph1pRNAs include a complementary oligonucleotide (DNA tag) annealed to the added sequence on the 3′ end.

FIG. 59 shows the inhibition of luciferase expression by various chimeric pRNA/siRNA using a dual luciferase assay.

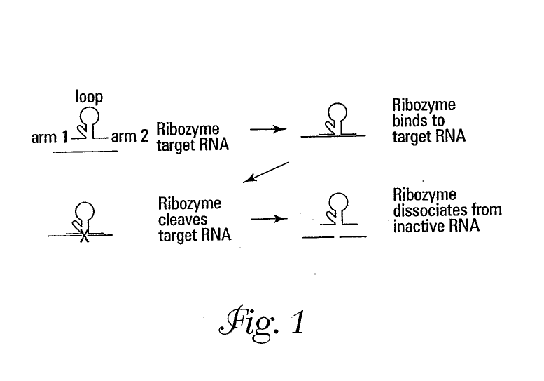

### DETAILED DESCRIPTION

Bacteriophage φ29 (phi29) is a double-stranded DNA virus. In 1987, one of the inventors, Dr. Peixuan Guo, discovered a viral-encoded 120 base RNA that plays a key role in bacteriophage φ29 DNA packaging (Guo et al. Science 236:690-694 (1987)). This RNA is termed packaging RNA or “pRNA”. It binds to viral procapsids at the portal vertex (the site where DNA enters the procapsid) (Guo et al., Nucl. Acids Res. 15:7081-7090 (1987)) and is not present in the mature φ29 virion.

Six copies of pRNA are needed to package one genomic DNA (Trottier et al., J. Virol. 70:55-61 (1996); Trottier et al., J. Virol. 71, 487-494 (1997); Guo et al., Mol Cell. 2, 149-155 (1998)). DNA packaging is completely blocked when one of the six slots is occupied by one inactive pRNA with a mutation at the 5′ or 3′ end (Trottier et al., J. Virol. 70:55-61 (1996); Trottier et al., J. Virol. 71:487-494 (1997)). Bacteriophage φ29 pRNA is associated with procapsids during the DNA translocation process (Chen et al., J. Virol. 71:3864-3871 (1997)). Inhibition data also suggests that the pRNA plays an essential role in DNA translocation (Trottier et al., J. Virol. 71:487-494 (1997)); Trottier et al. J. Virol70:55-6 (1996)). A Mg²⁺-induced conformational change of pRNA leads to its binding to the portal vertex (Chen et al. J. Virol. 71, 495-500 (1997)). The tertiary structure of the pRNA monomer and dimer has also reported (Zhang et al., Virology 81:281-93 (2001); Trottier et al., RNA 6(9):1257-1266 (2000); Chen et al. J. Biol. Chem. 275(23): 17510-17516 (2000); Garver et al., J. Biol. Chem. 275(4): 2817-2824 (2000)).

Recently, a computer model of the three-dimensional structure of a pRNA monomer has been constructed (Hoeprich and Guo, J. Biol. Chem. 277:20794-803 (2002)) based on experimental data derived from photo-affinity cross-linking (Garver and Guo, RNA 3:1068-79 (1997); Chen and Guo, J Virol 71:495-500(1997)); chemical modification and chemical modification interference (Mat-Arip et al., J Biol Chem 276:32575-84 (2001); Zhang et al., Virology 281:281-93 (2001); Trottier et al., RNA 6:1257-66 (2000)); complementary modification (Zhang et al., RNA 1:1041-50 (1995); Zhang et al., Virology 201:77-85 (1994); Zhang et al., RNA 3:315-22 (1997); Reid et al., J Biol Chem 269:18656-61 (1994); Wichitwechkarn et al., Mol Biol 223:991-98 (1992)); nuclease probing (Chen and Guo, J Virol 71:495-500 (1997); Reid et al., J Biol Chem 269:5157-62 (1994); Zhang et al., Virology 211:568-76 (1995)); oligo targeting competition assays (Trottier and Guo, J Virol 71:487-94 (1997); Trottier et al., J Virol 70:55-61(1996)) and cryo-atomic force microscopy (Mat-Arip et al., J Biol Chem 276:32575-84 (2001); Trottier et al., RNA 6:1257-66 (2000); Chen et al., J Biol Chem 275:17510-16 (2000)). pRNA hexamer docking with the connector crystal structure reveals a very impressive match with available biochemical, genetic, and physical data concerning the 3D structure of pRNA (Hoeprich and Guo, J. Biol. Chem 277:20794-803 (2002)).

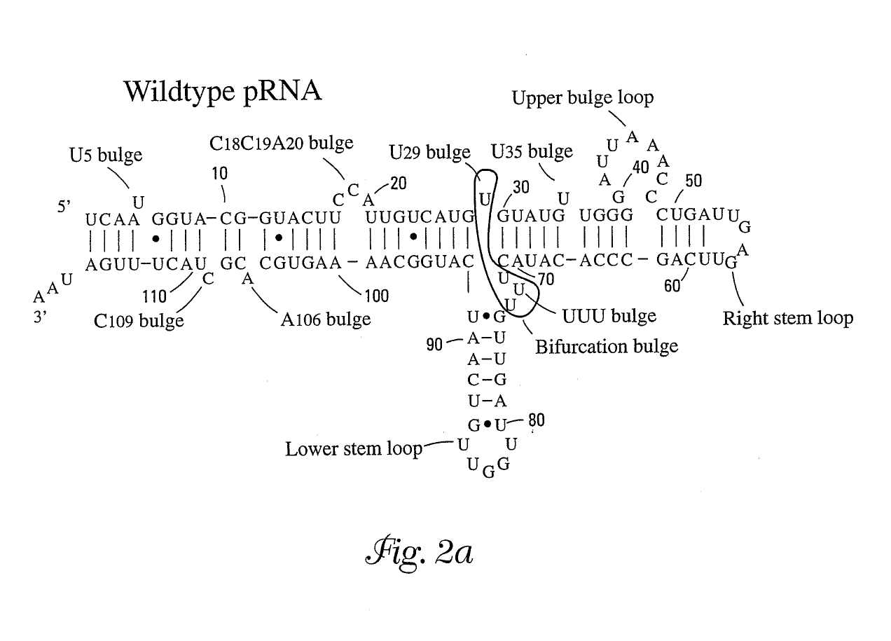

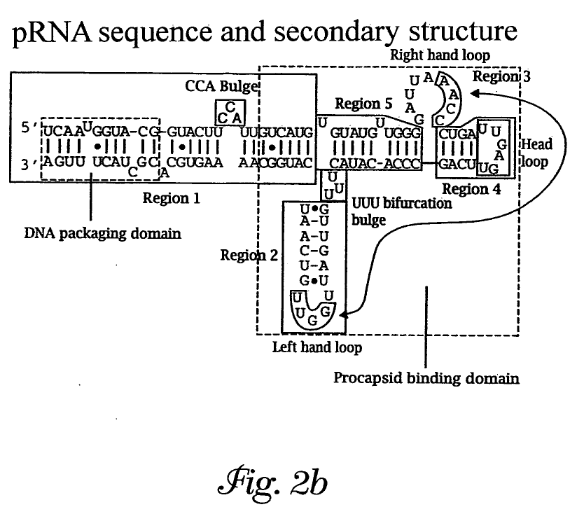

The nucleotide sequence (SEQ ID NO:1) of native full length φ29 pRNA (Guo et al., Nucl. Acids Res. 15:7081-7090 (1987)), as well as its predicted base-paired secondary structure, is shown in FIG. 2(a) (Zhang et al., RNA 3:315-323 (1997); Zhang et al., Virology 207:442-451 (1995)). The predicted secondary structure has been partially confirmed (Zhang et al., RNA 1:1041-1050 (1995); Reid et al., J. Biol. Chem. 269:18656-18661 (1994); Zhang et al., Virology 201:77-85 (1994); Chen et al., J. Virol. 71: 495-500 (1997)).

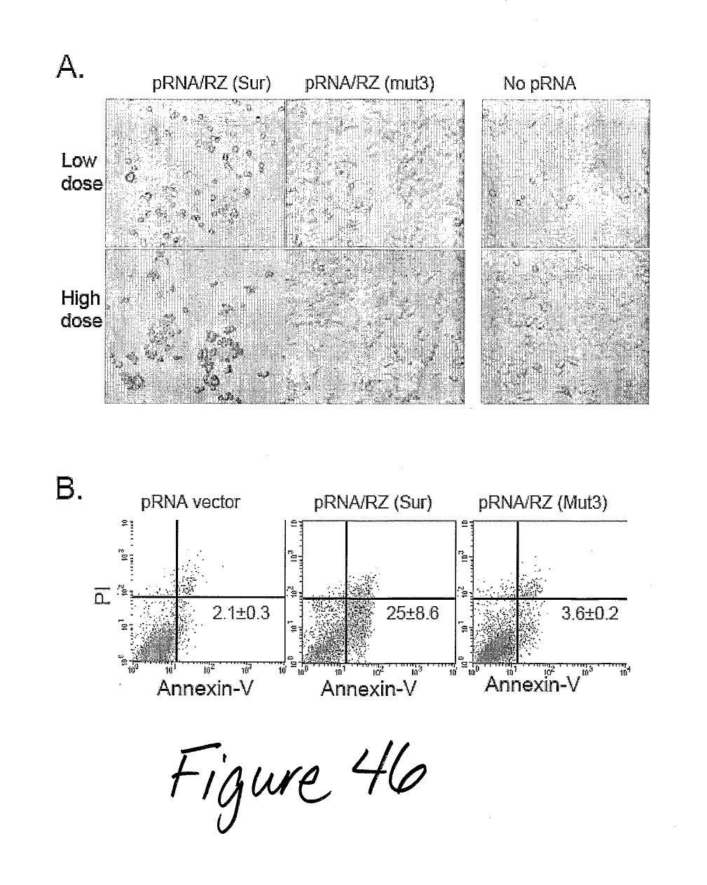

As shown in FIG. 2(b), the pRNA monomer contains two functional domains. One domain is the procapsid binding domain. This domain is inclusive of the “intermolecular interaction domain” (see, e.g., FIG. 45) that facilitates the interactions (e.g., dimerization, trimerization) of pRNA molecules. This domain includes a right hand loop and a left hand loop that are important in the interaction between pRNAs, as described in more detail below. The other domain is the DNA translocating domain, also referred to herein as the 5′/3′ double-stranded (paired) helical domain. The procapsid binding domain is located at the central part of the pRNA molecule at bases 23-97 (Garver et al., RNA 3:1068-79 (1997); Chen et al., J Biol Chem 275:17510-16 (2000)), while the DNA translocation domain is located at the 5′/3′ paired ends. The 5′ and 3′ ends have been found to be proximate, and several kinds of circularly permuted pRNA have been constructed (Zhang et al., RNA 3:315-22 (1997); Zhang et al., Virology 207:442-51 (1995); Guo, Prog in Nucl Acid Res & Mole Biol 72:415-72 (2002)). These two domains are compact and fold independently, suggesting that exogenous RNA can be connected to the end of the pRNA without affecting pRNA folding and that phi29 pRNA could be used as a vector to escort and chaperone small therapeutic RNA molecules. Indeed, removal of the DNA translocating domain does not change the nature of pRNA's intermolecular interaction, i.e., replacement or insertion of nucleotides before residue #23 or after residue #97 does not interfere with the formation of dimers, trimers, and hexamers (Hoeprich et al., Gene Therapy, 10(15):1258-1267 (2003); Chen et al., RNA, 5:805-818 (1999); and Shu et al., J Nanosci and Nanotech (JNN), 4:295-302 (2003)). We have confirmed that exogenous RNA can be connected to the 3′ or 5′ end of the pRNA without affecting pRNA folding; this foreign RNA molecule also folds independently (Hoeprich et al., Gene Therapy, 10(15):1258-1267 (2003); Shu et al., J Nanosci and Nanotech (JNN), 4:295-302 (2003); Guo, J. Nanosci Nanothechnol, 2005, 5(12):1964-1982).

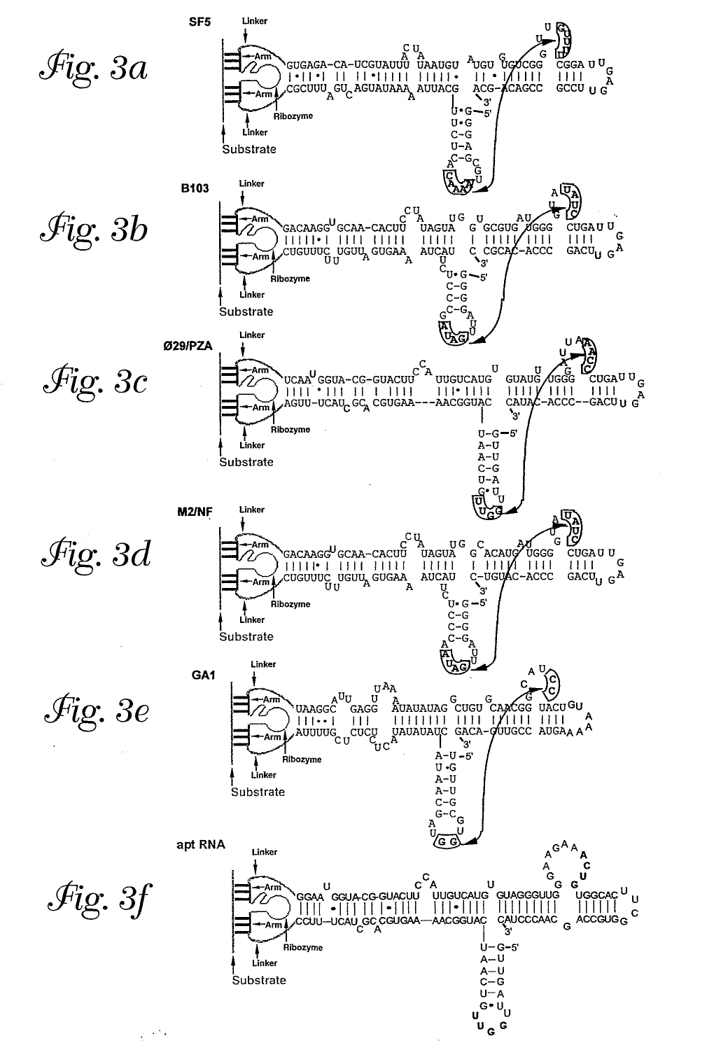

Phylogenetic analysis of pRNAs from phages SF5′, B103, φ29, PZA, M2, NF and GA1 (Chen et al., RNA 5:805-818 (1999)) shows very low sequence identity and few conserved bases, yet the family of pRNAs appear to have strikingly similar and stable predicted secondary structures (FIG. 3). The pRNAs from bacteriophages SF5′ (SEQ ID NOS:11 and 28), B103 (SEQ ID NOS:12 and 29), φ29/PZA (SEQ ID NOS:13 and 30), M2/NF (SEQ ID NOS:14 and 31), GA1 (SEQ ID NOS:15 and 32) of Bacillus subtilis (Chen et al., RNA 5:805-818 (1999); and aptRNA (SEQ ID NOS:16 and 33) are all predicted to have a secondary structure that exhibits essentially the same structural features as shown in FIG. 2 for φ29 pRNA (Chen et al., RNA 5:805-818 (1999)). All have native 5′ and 3′ ends at the left end of a stem structure (as shown in FIG. 3) and contain the same structural features positioned at the same relative locations.

The pRNA of these bacteriophages, sharing as they do a single stable secondary structure, provide the framework for the pRNA chimera of the invention.

Secondary structure in an RNA molecule is formed by base pairing among ribonucleotides. RNA base pairs commonly include G-C, A-T and U-G. Predictions of secondary structure are preferably made according to the method of Zuker and Jaeger, for example by using a program known by the trade designation RNASTRUCTURE 3.6, written by David H. Mathews (Mathews et al., J. Mol. Biol. 288:911-940 (1999); see also Zuker, Science 244:48-52 (1989); Jaeger et al., Proc. Natl. Acad. Sci. USA 86:7706-7710 (1989); Jaeger et al., Meth. Enzymol. 183:281-306 (1990)). This program is publicly available on the worldwide web at the homepage of the laboratory of Douglas Turner at the University of Rochester at ma.chem.rochester.edu/RNAstructure.html and runs on MS Windows 95, 98, ME, 2000 and NT4. The program is also publicly available on the worldwide web at Michael Zuker's homepage at Rensselaer Polytechnic Institute (bioinfo.math.rpi.edu/˜zukerm/home.html); his homepage offers online folding and a version of the algorithm that can be compiled on Silicon Graphics, Sun, or DEC Alpha workstations. The structure with the lowest energy (i.e., the optimal structure) is chosen.

Secondary structures of RNA can be characterized by stems, loops and bulges. A “stem” is a double-stranded section of two lengths of base-paired ribonucleotides. Stem sections contain at least 2 base pairs and are limited in size only by the length of the RNA molecule. A “loop” is a single-stranded section that typically includes at least 3 ribonucleotides and is also limited in size only by the length of the RNA molecule. In a “stem loop”, the 5′ and 3′ ends of the loop coincide with the end of a base-paired stem section. In a “bulge loop”, the loop emerges from along the length of a stem section. The 5′ and 3′ ends of a bulge loop are typically not base paired although they may potentially be (see, e.g., G40 and C48 of the bulge loop in the φ29 pRNA structure; FIG. 2). A “bulge” is an unpaired single stranded section of about 1 to about 6 ribonucleotides present along the length of (or between) stem sections. Note that there is no clear line between a large “bulge” and a small “bulge loop.” Herein, where the term “bulge” is used, it also includes a small “bulge loop” (i.e., a bulge loop of less than about 7 ribonucleotides).

The secondary structure of an RNA molecule is determined by the nature and location of the base pairing options along its length. RNA secondary structure is degenerate; that is, different primary ribonucleotide sequences can yield the same base pairing configurations and hence the same secondary structure. In a way, it is akin to the way multiple amino acid sequences can produce the same secondary structure, for example an α-helix.

A single secondary structure is dictated by a number of different primary sequences in predictable and well-understood ways. For example, single or pairs of nucleotides can generally be added, removed, or substituted without altering the overall base pairing interactions within the RNA molecule and without interfering with its biological function. This is particularly true if one or a few base pairs of nucleotides are removed, added or substituted along double-stranded hybridized length of the molecule, or if one or more nucleotides are removed, added or substituted in the single-stranded loop regions. For example, although GC base pairs and AT base pairs differ slightly in their thermodynamic stability, one can generally be substituted for another at a site within the double-stranded length without altering the secondary structure of an RNA molecule. GC base pairs are preferred in the stem region due to their added stability. Changes in secondary structure as a result of addition, deletion or modification of nucleotides can be readily assessed by applying the secondary structure prediction algorithm of Zuker and Jaeger as described above. The 5′/3′ double-stranded helical region of the pRNA can accommodate substantial variation in primary sequence without an appreciable change in secondary structure.

The pRNA chimera of the invention is useful as a vehicle to carry and deliver one or more biologically active moieties, detectable labels, and the like to a target molecule, cell or location. The biologically active moieties, detectable labels and the like are considered herein as “heterologous” components of the pRNA chimera (that is, they are not present in the naturally occurring pRNA) and are sometimes referred herein the “cargo” carried by the pRNA chimera. Heterologous components of pRNA chimera of the invention that are, like the pRNA itself, RNAs are sometimes referred to herein as “daughter” RNAs. The cargo components can be oligonucleotides, polynucleotides, peptides, polypeptides, carbohydrates, lipids, hormones, labeling agents, small organic molecules, and the like, without limitation, and any pRNA that has been derivatized with, conjugated to, or otherwise contains or is associated with a cargo component is considered a “pRNA chimera” or a “chimeric pRNA” of the invention. As described in more detail below, the chimeric pRNAs can advantageously be considered as “building blocks” that can be customized, selected, mixed and matched to produce multimeric, polyvalent pRNA complexes tailor-made for a desired application or purpose.

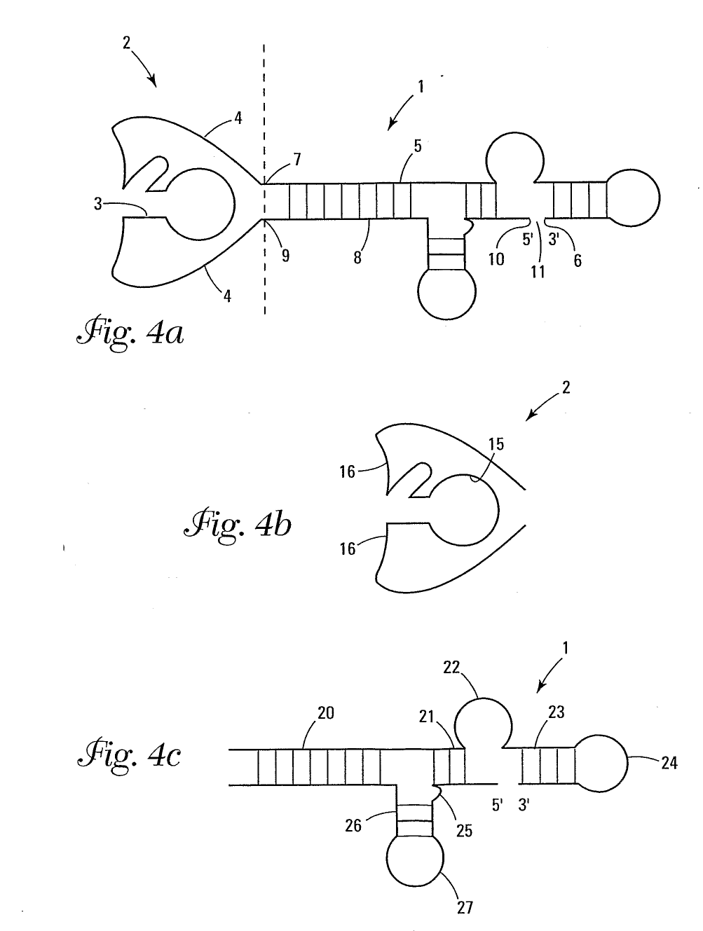

The pRNA chimera of the invention can take either of two general forms. In a one embodiment, the pRNA chimera consists essentially of a pRNA region having the secondary structure exemplified in FIG. 3 (and schematically depicted in FIG. 4, as detailed below), interrupted by (i.e., flanking) a heterologous spacer region that contains a biologically active moiety, preferably an RNA such as an RNA aptamer for targeting to a cell-surface receptor or a ribozyme. This embodiment of the pRNA chimera, which contains a heterologous spacer region attached to the naturally occurring 5′ and 3′ ends, has its 5′ and 3′ ends at non-native positions and represents a “circular permutation” of the RNA sequence when compared to naturally occurring pRNA, as described in more detail below.

The secondary structure of the pRNA region of the pRNA chimera is the common secondary structure that characterizes the pRNA from bacteriophages φ29, SF5′, B103, PZA, M2, NF and GA1. The spacer region is termed “heterologous” because all or a portion of its nucleotide sequence is engineered or it is obtained from an organism other than the bacteriophage. It is the presence of the heterologous spacer region that renders the construct “chimeric” for the purposes of this invention. Since both ends of the cargo RNA are connected to pRNA, the linkage is expected to protect sensitive cargo, such as a ribozyme, from degradation and to assist the biologically active moiety to fold appropriately.

Notably, the ability of the pRNA chimera to perform its intended function of protecting and carrying a biologically active moiety depends not on the primary nucleotide sequence of the pRNA region (the primary structure), but on the secondary structure (base pairing interactions) that the pRNA region assumes as a result of its primary ribonucleotide sequence. The “pRNA region” of the pRNA chimera is so termed because it has a secondary structure, although not necessarily an RNA sequence, characteristic of a native bacteriophage pRNA molecule. Therefore, unless otherwise specified, the term “pRNA region” as used herein includes naturally occurring (native) pRNA sequences, nonnaturally occurring (nonnative) sequences, and combinations thereof provided that they yield the secondary structure characteristic of naturally occurring (native) bacteriophage pRNA as described herein. Stated another way, the term “pRNA region” is not intended to be limited to only those particular nucleotide sequences native to pRNA. The pRNA region can thus contain any nucleotide sequence which results in the secondary structure shown in FIG. 4. Nucleotide sequences that fold into the aforesaid secondary structure include naturally occurring sequences, those that are derived by modifying naturally occurring pRNA sequences, and those that are designed de novo, as well as combinations thereof. One of skill in the art can readily determine whether a nucleotide sequence will fold into the secondary structure shown in FIG. 4 and described herein by applying a secondary structure algorithm, such as RNASTRUCTURE as described above, to the nucleotide sequence.

Examples of nucleotide sequences that, when folded, yield the secondary structure of the pRNA region of the pRNA chimera of the invention are shown in FIG. 3. They include pRNA sequences from bacteriophages SF5′ (SEQ ID NOS:11 and 28), B103 (SEQ ID NOS:12 and 29), φ29/PZA (SEQ ID NOS:13 and 30), M2/NF (SEQ ID NOS:14 and 31), GA1 (SEQ ID NOS:15 and 32) as well as the aptRNA (SEQ ID NOS:16 and 33).

In embodiments of the circularly permitted pRNA chimera wherein the pRNA region includes or is derived from a naturally occurring pRNA, the spacer region of the pRNA chimera is covalently linked to the pRNA region at what can be considered the “native” 5′ and 3′ ends of a pRNA sequence, thereby joining the native ends of the pRNA region. The pRNA region of the pRNA chimera is optionally truncated when compared to the native bacteriophage pRNA; in those embodiments, and that as a result the “native” 5′ and 3′ ends of the pRNA region simply refer to the nucleotides that terminate or comprise the actual end of the truncated native pRNA. An opening is formed in the pRNA region to linearize the resulting pRNA chimera, effecting a “circular permutation” of the pRNA as detailed below. It should nonetheless be understood that the term “circularly permuted pRNA region” is not limited to naturally occurring pRNAs that have been circularly permuted but instead is intended to have the broader meaning of RNA having a pRNA-like secondary structure as shown in FIG. 4(c), including an opening in the pRNA region that forms the 5′ and 3′ ends of the pRNA chimera.

Examples of pRNA chimera of the invention are those formed from the pRNAs of bacteriophages SF5′ (SEQ ID NOS: 11 and 28), B103 (SEQ ID NOS:12 and 29), φ29/PZA (SEQ ID NOS: 13 and 30), M2/NF (SEQ ID NOS:14 and 31), GA1 (SEQ ID NOS:15 and 32) as well as aptRNA (SEQ ID NOS:16 and 33) by joining the native 5′ and 3′ ends to the spacer region and introducing an opening elsewhere in the pRNA region, as described herein. Another example of a pRNA chimera of the invention is: 5+-GUUGAUNⱼGUCAAUCAUGGCAA-spacer region-UUGUCAUGUGUAUGUUGGGGAUUANⱼCUGAUUGAGUUCAGCCCAC AUAC-3′ (SEQ ID NOS: 45 and 7 respectively) where N represents any nucleotide, without limitation and j is an integer between about 4 to about 8. Preferably j is 4 or 5. The spacer region is represented by Nₘ—B—Nₙ where Nₙ and Nₘ are nucleotide strings that are optionally included in the spacer region, and B includes the biologically active moiety. Preferably, B is a ribonucleotide sequence that includes a biologically active RNA. Both m and n can be independently zero or any integer. Preferably, m and n are independently at least about 3, more preferably at least about 5, and most preferably at least about 10. Further, n and m are independently preferably at most about 300, more preferably at most about 50, and most preferably at most about 30.

Further, since the pRNA region of the pRNA chimera is defined by its secondary structure, still other examples of a pRNA chimera can be readily made by “mixing and matching” nucleotide fragments from, for example, SEQ ID NO:s 1, 2, 7, 11, 12, 14, 15 and 16 that fold into particular secondary structural features (bulges, loops, stem-loops, etc.) provided that the resulting nucleotide sequence folds into the overall secondary structure as shown in FIG. 4. For example, nucleotides encoding bulge loop 22 from bacteriophage SF5′ pRNA (SEQ ID NO:11) could be substituted for the nucleotides encoding bulge loop 22 in the φ29 pRNA (SEQ ID NO:1) to yield a pRNA region as described herein. Likewise, any number of artificial sequences can be substituted into SEQ ID NO:s 1, 2, 7, 11, 12, 14, 15 and 16 to replace nucleotide sequences that fold into one or more structural features (or portions thereof) to form a pRNA region as described herein. See, for example, aptRNA (FIG. 3(f)) which was derived in that fashion from φ29 pRNA. The overarching principle is that the overall secondary structure of the pRNA region is the secondary structure common to the bacteriophage pRNAs, as schematically depicted in FIG. 4.

Importantly, the pRNA chimera embodiment that is circularly permuted is not a circular molecule; rather, it is linearized due to a circular permutation of the pRNA region (Zhang et al., RNA 3:315-323 (1997); Zhang et al., Virology 207:442-451 (1995)). Briefly, an opening (i.e., a cleavage or break point) is provided in the pRNA region at any designated site to form the actual 5′ and 3′ ends of the RNA chimera. These 5′ and 3′ ends are at “nonnative” positions with respect to a naturally occurring linear pRNA.

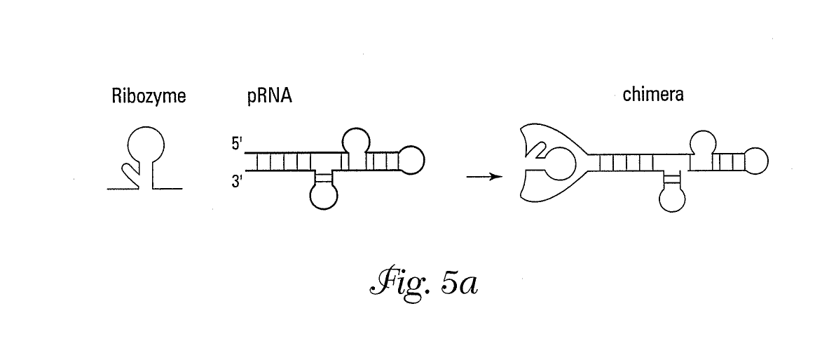

FIG. 5(a) shows how a pRNA chimera of the invention can be formed from a ribozyme and a pRNA region. The 5′ and 3′ ends of the pRNA can be engineered into any desired site on the circularly permuted pRNA chimera. FIG. 5(b) shows exemplary circularly permuted RNA molecules showing various locations for the circle openings.

FIG. 4 depicts various structural features that characterize a circularly permuted pRNA chimera of the invention. As shown in FIG. 4(a), the linear molecule includes a pRNA region 1 and, in the case of a pRNA chimera that is circularly permuted, a spacer region 2. Spacer region 2 contains a biologically active moiety 3, in this case a ribozyme, flanked by ribonucleotide strings 4. The pRNA region 1 is bifurcated; it includes a first pRNA segment 5 having 3′ end 6 and “native” 5′ end 7, and a second pRNA segment 8 having “native” 3′ end 9 and 5′ end 10. Ends 6 and 10 are the actual terminal ends of the pRNA chimera. Opening 11 renders the molecule linear and can be positioned anywhere in pRNA region 1 by the relocation of ends 6 and 10.

Spacer region 2 is shown in detail in FIG. 4(b). Ribozyme 3 is composed of a catalytic domain 15 flanked by target-binding sequences 16.

pRNA region 1 is shown in detail in FIG. 4(c). Overall, pRNA region 1 is characterized by a stem-loop secondary structure, wherein the head loop, loop 24 is relatively small and the base-pairing in the stem (essentially stem sections 20, 21 and 23) is interrupted by structures on either side of loop 24. Bulge loop 22, the “right hand loop” is positioned 5′ of loop 24. Positioned 3′ of loop 24 is a stem-loop structure that contains bulge 25, stem 26 and loop 27, the “left hand loop”.

Stem section 20 can be any number of ribonucleotides in length and can contain an unlimited number of bulges provided it is still able to base pair. Preferably, stem section 20 contains at least about 4, more preferably at least about 10 base pairs; further, it preferably it contains at most about 50, more preferably at most about 40 base pairs. Preferably stem section 20 contains about 0 to about 8 bulges; more preferably it contains about 0 to about 4 bulges.

Note that in non-circularly permuted embodiments of the pRNA chimera of the invention, described in more detail below, stem section 20 can be replaced by a double-stranded siRNA. In this embodiment of the chimeric pRNA, the “cargo” carried by the chimeric pRNA takes the form of stem section 20 itself, which constitutes biologically active siRNA. As shown in Examples 11 and 12, there is evidence that upon delivery to the cell, the siRNA is cleaved from the pRNA molecule and is effective to silence the target gene.

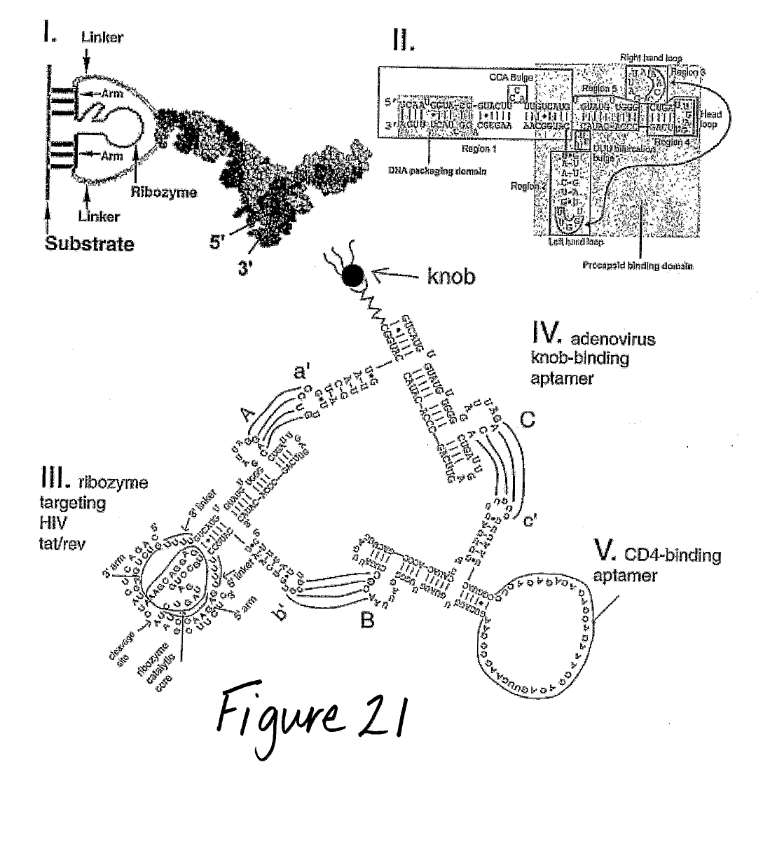

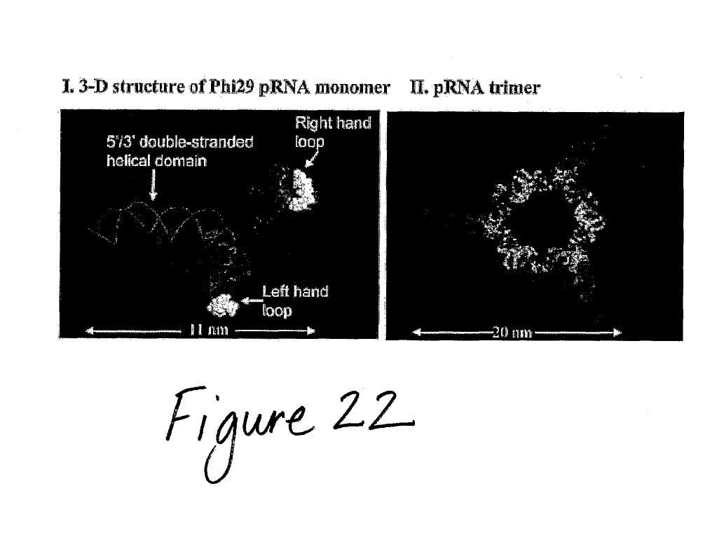

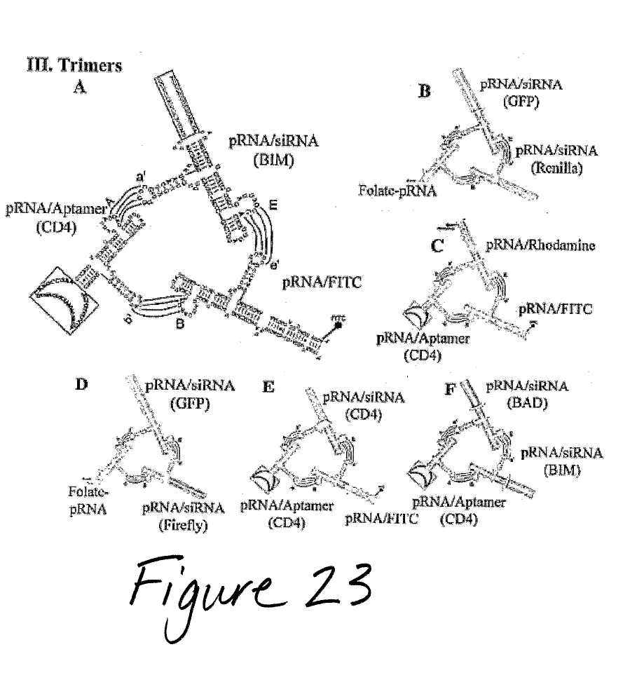

In other embodiments of non-circularly permuted chimeric pRNA, which embodiments by virtue of not being circularly permuted exhibit 5′ and 3′ ends at or near their “native” positions, stem section 20 can be derivatized at either or both of said 5′ or 3′ ends with a biologically active moiety, detectable label, or the like, as its heterologous component “cargo”. For example, the 5′ end of the pRNA present in stem section 20 can be derivatized with folate (to facilitate targeting) or with a fluorescent label (to facilitate detection). See Example 11 and FIG. 23 for examples of various stem sections 20 that can be employed in forming a pRNA chimera of the invention.

Stem section 21 preferably contains 5-13 base pairs and 0-2 bulges.

Bulge loop 22 preferably contains 5-12 bases.

Stem section 23 preferably contains 3-12 base pairs and 0-2 bulges.

Loop 24 preferably contains 3-8 bases.

Bulge 25 preferably contains 0-5 bases.

Stem 26 preferably contains 4-8 base pairs and 0-2 bulges.

Loop 27 preferably contains 3 -10 bases.

Tertiary interactions within an RNA molecule may result from nonlocal interactions of areas of the RNA molecule that are not near to each other in the primary sequence. Although native bacteriophage pRNA appears to exhibit tertiary interactions between bulge loop 22 (the “right hand” loop) and loop 27 (the “left hand” loop) (Chen et al., RNA 5:805-818 (1999); Guo et al, Mol. Cell. 2:149-155 (1998)), it should be understood that the pRNA chimera of the invention is not limited to RNA molecules exhibiting any particular tertiary interactions. On the other hand, it will be appreciated that these intramolecular, tertiary interactions can be used to advantage. For example, in the mutimeric pRNA complexes provided by the invention, the interactions between the right and left hand loops of the various monomers can be controlled by engineering in the desired complementarity, advantageously resulting in customized dimers, trimers, and hexamers for use in therapeutic applications; see Example 7, for instance.

In one embodiment, the pRNA chimera of the invention contains at least 8, more preferably at least 15, most preferably at least 30 consecutive ribonucleotides found in native SF5′ pRNA (FIG. 3(a)), B103 pRNA (FIG. 3(b)), φ29/PZA pRNA (FIG. 3(c)), M2/NF pRNA (FIG. 3(d)), GA1 pRNA (FIG. 3(e)), or aptRNA (FIG. 3(f)), preferably native φ29 pRNA. Most preferably, the pRNA region of the pRNA chimera contains at least a φ29 pRNA sequence that starts at ribonucleotide 23, preferably at ribonucleotide 20, and ends at ribonucleotide 95, preferably ribonucleotide 97, in the φ29 pRNA sequence (FIG. 2). In addition or in the alternative, the nucleotide sequence of the pRNA region of the pRNA chimera is preferably at least 60% identical to, more preferably 80% identical to, even more preferably 90% identical to, and most preferably 95% identical to the nucleotide sequence of a corresponding native SF5′ pRNA (FIG. 3(a)), B103 pRNA (FIG. 3(b)), φ29/PZA pRNA (FIG. 3(c)), M2/NF pRNA (FIG. 3(d)), GA1 pRNA (FIG. 3(e)), or the aptRNA chimera (FIG. 3(f)), most preferably φ29 pRNA (particularly bases 20-97).

Percent identity is determined by aligning two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. For example, the two nucleotide sequences are readily compared using the Blastn program of the BLAST 2 search algorithm, as described by Tatusova et al. (FEMS Microbiol Lett 1999, 174:247-250). Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on.

In circularly permuted embodiments of the pRNA chimera, the covalent linkages between the biologically active moiety and the pRNA region can be direct or indirect but preferably are indirect. In an indirect linkage, the spacer region includes additional string(s) of ribonucleotides at one or both ends of the biologically active moiety. These ribonucleotide strings, if present, contain preferably at least about 3 ribonucleotides; and preferably contain at most about 300, more preferably at most about 30 ribonucleotides. Compositionally, the strings can contain any desired ribonucleotides, however it is preferably that ribonucleotide compositions are selected so as to prevent the ribonucleotide strings on either side of the biological moiety from base pairing with each other or with other parts of the pRNA chimera.

Exemplary biologically active moieties include, without limitation, DNA, RNA, DNA or RNA analogs, including a ribozyme, a siRNA, an RNA aptamer, or an antisense RNA, peptide nucleic acid (PNA), a peptide, a protein such as an antibody, a polysaccharide, a lipid, a virus, a plasmid, a cofactor, or a combination thereof. Biologically active moieties can be selected without limitation, and include those having desired activity or characteristic, such as binding activity, enzymatic activity, and the like. Preferably the biological activity of the biologically active moieties is an enzymatic activity or binding activity or both; for example, the biologically active moiety may function as or encode a ribozyme or other catalytic moiety. Since siRNA is a double-stranded RNA, the effective siRNA moiety could include any sequence to replace the 5′/3′ paired helical region, instead of being positioned in the spacer region of a circularly permuted pRNA chimera, as described in more detail below.

The biologically active moiety is preferably a polynucleotide. A preferred biologically active polynucleotide is a polyribonucleotide, more preferably the biologically active polynucleotide is a ribozyme such as a hammerhead ribozyme or a hairpin ribozyme. Antisense RNA and other bioactive RNAs are also preferred.

It should be understood that the terms “nucleotide,” “oligonucleotide,” and “polynucleotide” as used herein encompass DNA, RNA, or combinations thereof, unless otherwise indicated. Further, the terms DNA and RNA should be understood to include not only naturally occurring nucleic acids, but also sequences containing nucleotide analogs or modified nucleotides, such as those that have been chemically or enzymatically modified, for example DNA phosphorothioates, RNA phosphorothioates, and 2′-O-methyl ribonucleotides.

In a preferred embodiment of a pRNA chimera of the invention, one or more nucleotide derivatives, such as 2′-NH₂-2′-deoxy CTP, 2′-CH₃-2′-deoxy CTP, 2′-F-2′ deoxy CTP, 2′-F-2′ deoxy UTP, and spiegelmers (L-nucleotide aptamers) are incorporated into the pRNA during synthesis to produce stable RNA transcripts that are resistant to RNase digestion. The stabilizing modification is preferably made at the 2′ position of the ribonucleotide or at other positions. In most cases, incorporation of the stablizing nucleotide derivatives is not expected to significantly interfere with dimerization or trimerization of the pRNAs to form a multimeric complex, nor is it expected to adversely impact the activity or function of the “cargo” moiety. Since biological function of the pRNA itself (other than its ability to form multimeric complexes) is not a concern, inclusion of non-natural nucleotide derivatives is suitable, especially for the receptor-binding aptamers selected from a random pool (e.g., using SELEX). If the incorporation of RNase-resistant nucleotide derivatives into a therapeutic RNA tethered to the pRNA does happen to interfere with the biological activity, such as a catalytic function, of a cargo RNA, for example, the cargo RNA can be synthesized with regular nucleotides and ligated to the pRNA molecule.

A ribozyme is generally characterized by:
arm 1—active enzyme center—arm 2
   where arm 1 and arm 2 are sequences complementary to the target substrate to be cleaved by the ribozyme, and the active enzyme center is the catalytic center that cleaves the target RNA. The “arms” of the ribozyme typically contain at least about 7 nucleotides, preferably at least about 12 nucleotides; and typically contain at most about 100 nucleotides, preferably at most about 30 nucleotides. The nucleotide sequence of the arms can be engineered to hybridize to the nucleotide sequence of any desired target nucleic acid.

Advantageously, incorporating a biologically active polynucleotide, e.g., a ribozyme, into the circularly permuted pRNA chimera of the invention protects the ends of the ribozyme thereby rendering it resistant to exonuclease degradation. Moreover, the secondary structure of pRNA is compact and very stable. A pRNA domain defined by nucleotides 30-91 of φ29 pRNA is especially stable.

The compactness and stability of pRNA allows the pRNA region and the ribozyme to fold independently. Proper folding of the inserted RNA is facilitated, thereby preserving its biological activity. The stable structure of the carrier pRNA region is retained as well. A major obstacle in designing molecules to deliver ribozymes, i.e., misfolding of the ribozyme and carrier region as a result of interactions between them, has thus been overcome by utilizing the very stable pRNA molecule as the carrier. That the activity of the ribozyme is retained in the circularly permuted pRNA chimera is especially significant because it means that the new 5′ and 3′ ends of the pRNA chimera can be positioned so as to “bury” them in the folded pRNA structure, thereby further protecting the pRNA chimera from degradation. These features suggest great promise for the use of the pRNA chimera of the invention as a ribozyme delivery vehicle in medical and veterinary applications.

As shown in the Examples below, circularly permuted pRNAs were constructed without impacting pRNA folding. In addition, connecting the pRNA 5′/3′ ends with variable sequences did not disturb its folding and function. These unique features, which help prevent two common problems—exonuclease degradation and misfolding in the cell, make pRNA an ideal vector to carry therapeutic RNAs.

As already noted, in one embodiment the pRNA chimera of the invention employs a “circular permutation” of a bacteriophage pRNA. A “circularly permuted” RNA molecule (cpRNA) is a linear RNA molecule in which the native 5′ and 3′ ends are covalently linked. The linkage can be direct, or it can be indirect by using a spacer region. Since a cpRNA molecule is linear, new nonnative 5′ and 3′ ends are created by forming an opening in the molecule (i.e., a discontinuity in the pRNA sequence) at a different location. The pRNA chimera of the invention is linear as a result of a nonnative opening in the bacteriophage pRNA framework at a designated site, which circularly permutes the bacteriophage framework and forms the actual 5′ and 3′ ends of the pRNA chimera. As already noted, the nonnative opening can be at any desired location in the pRNA region. Examples of selected locations in, for example in φ29 pRNA can be found in Zhang et al., RNA 3:315-323 (1997) and Zhang et al., Virology 207:442-451 (1995). See also Garver et al., J. Biol. Chem. 275:2817-2824 (2000); Chen et al., J. Virology 71:495-500 (1997); Trottier et al., RNA 6:1257-1266 (2000); and Zhang et al., Virology 281:281-293 (2001).

The pRNA chimera of the invention can be synthesized chemically or enzymatically using standard laboratory protocols. The pRNA region is preferably transcribed from a DNA template that encodes it, although if desired it can be synthesized chemically. Alternatively, the chimeric pRNA can be assembled from RNA fragments (modular components) that have been chemically synthesized (see, e.g., Fang et al., Biochemistry, 2005, 44(26):9348-9358). If synthesized chemically, the pRNA region optionally contains nonnative nucleotides (e.g., derivatized or substituted nucleotides) and/or nonnative bonds analogous to the phosphodiester bonds that characterize naturally occurring nucleic acids. The inclusion of nonnative nucleotides or nonnative bonds can increase the stability of the pRNA chimera and make it more resistant to enzymatic degradation.

Preferably the pRNA region is transcribed or synthesized as a single RNA molecule. In one embodiment of the method as it applies to the creation of a circularly permuted pRNA, the spacer region is chemically or enzymatically linked to the “native” ends of the pRNA region to form a circular chimeric molecule. The pRNA is then cleaved at a predetermined site to form the linear, circularly permuted pRNA chimera.

When the spacer region is RNA, another embodiment of the method as it applies to the creation of a circularly permuted pRNA includes transcribing the entire pRNA chimera from a single DNA template that encodes the entire chimeric molecule. In another embodiment of the method, the RNA spacer region is produced separately, either via transcription from its own template or by chemical synthesis, after which it is ligated to the pRNA region.

In addition to a circularly permuted pRNA chimera, which has the “cargo” moiety (e.g., RNA aptamer or ribozyme) positioned in the spacer region, the invention also provides another embodiment of a pRNA chimera wherein the “cargo” moiety is incorporated into or attached elsewhere in the pRNA structure. This embodiment of the invention, while constituting a linear pRNA like the circularly permuted embodiment, is not circularly permuted vis a vis native phi29 pRNA. Both embodiments (i.e., circularly permuted and non-circularly permuted pRNAs) are termed “chimeric pRNA” or “pRNA chimera”, provided they contain a heterologous “cargo”, as described in more detail above.

One example of a non-circularly permuted pRNA chimera is a pRNA that includes an siRNA in place of the 5′/3′ double-stranded paired helical region of pRNA (see FIG. 23 and the accompanying text in Example 11 for several examples). Replacement or insertion of nucleotides preceding residue #23 or following residue #97 has been shown not to interfere with the formation of dimers as long as the strands are paired (Chen et al., RNA 1999; 5:805-818). siRNAs are short (typically 19-25 nucleotides, but can be shorter or longer) and thus are ideally suited for incorporation into a pRNA to form the 5′/3′ paired helical region. It should be noted that a pRNA chimera containing siRNA as its 5′/3′ paired helical region is preferably not circularly permuted, but it may be; circular permutation of the resulting pRNA may not eliminate the activity of the siRNA (see Example 11) and therefore is optional. A pRNA containing siRNA as its 5′/3′ paired helical region folds properly and can be used in conjunction with other pRNA chimera as described herein to form a polyvalent dimer, trimer, or hexamer.

When replacing the 5′/3′ paired helical region of a pRNA with an siRNA, substantial tolerance has been observed concerning the positions in the pRNA sequence at which the siRNA strands can be attached. It has been shown, for example, that the addition or deletion of nucleotides at the 5′ end preceding nucleotide 23, and at the 3′ end following nucleotide 97, does not affect the correct folding of the intermolecular interaction (procapsid binding) domain (Zhang et al., RNA 1995; 1:1041-1050). For example, gene silencing was achieved using complementary siRNA attached at positions 29 and 91 to form the paired helical region, and with the siRNA attached at positions 21 and 99 to form the paired helical region (see Example 11). What is important in incorporating the siRNA as the “stem” (e.g., stem 20 in FIG. 4c) to form the pRNA chimera is that the siRNA not intrude into the intermolecular interaction region (containing the right hand and left hand loops) such that it interferes with the interactions of the right and left hand loops in the formation of dimers, trimers and hexamers.

Another example of a non-circularly permuted pRNA is a pRNA that has been derivatized at or near its “native” 5′ and/or 3′ end with a biologically active moiety, detectable label, or other moiety of interest. A preferred moiety for attachment at or near the 5′ or 3′ end of the pRNA is a targeting moiety, such as an antibody or a receptor ligand. For example, covalent linkage of a folate molecule (see Example 13) targets the modified pRNA to cells having on their cell surface a folate receptor. It should be noted that in another aspect, the invention encompasses a method for conjugating folate to RNA, preferably pRNA, as illustrated in Example 13.

Another moiety that can be attached at or near the 5′ and/or 3′ end of a pRNA is a detectable label. One or more 5′ or 3′ end of a pRNA or a pRNA chimera (e.g., a pRNA that incorporates an siRNA as the paired helical region) may also be derivatized to include a detectable label, such as a fluorescent label, a radioactive label, or a paramagnetic label. Labeling at least one component of a multimeric pRNA complex allows the complex to be detected. Likewise, a therapeutic agent, such as a radionuclide, can be linked at or near a 5′ or 3′ end of a pRNA, a pRNA chimera or a modified pRNA to effect treatment of a subject once the multimeric complex has bound to and/or been internalized by the target cell.

It should be understood that derivitization of a pRNA at or near its 5′ or 3′ end encompasses linking the cargo moiety to whatever nucleotide presently constitutes the 5′ or 3′ end of the pRNA. In other words, a pRNA may be truncated (at either the 5′ or 3′ ends) with respect to a naturally occurring pRNA, or it may have one or more additional nucleotides added to its 5′ and/or 3′ end when compared to a naturally occurring pRNA. In either event, what is contemplated by the invention is derivatization of the first and/or last nucleotides of the linear pRNA; i.e., the 5′ and/or the 3′ nucleotides of that particular pRNA molecule. Furthermore, it should be understood that the heterologous component can be linked either covalently or noncovalently to the pRNA at or near the 5′ or 3′ end of the pRNA. Preferably, the linkage is covalent, except in the case where a complementary oligonucleotide constitutes the heterologous component, as discussed elsewhere herein.

In a particularly preferred embodiment, the 5′ or 3′ end of the pRNA is an overhanging end; that is, it is unpaired and extends past the paired helical region by one or more bases, and the heterologous component is linked to the overhanging end. Preferably, the heterologous component is linked to a 5′ overhanging end.

In another embodiment the overhanging end itself constitutes the heterologous component, as when the pRNA has been engineered to include an antisense RNA as a 5′ or 3′ extension, preferably as a 3′ extension Further, it is contemplated that other nucleotide positions on the 5′/3′ paired helical region can, if desired, be derivatized with a cargo moiety without adversely impacting dimerization, trimerization, or activity of the cargo moiety.

In yet another embodiment of the pRNA chimera of the invention, the heterologous component includes an oligonucleotide that is annealed to the 5′ or 3′ end of the pRNA. The interactions between the oligonucleotide and the pRNA chimera are preferably non-convalent; that is, the two molecules are associated via base-pairing interactions (e.g., hybridization). Preferably, the oligonucleotide is a DNA oligonucleotide although it may be an RNA oligonucleotide. In a particularly preferred embodiment, the pRNA has a 5′ or a 3′ overhanging end, and the oligonucleotide is annealed to the overhanging end, preferably the 3′ overhanging end. Optionally, the annealed oligonucleotide includes a detectable label, such as a biotin or a radiolabel. The oligonucleotide is preferably at least 5 nucleotides in length, more preferably at least 10 nucleotides in length, and most preferably at least 15 nucleotides in length. The oligonucleotide is preferably at most 70 nucleotides in length, more preferably at most 50 nucleotides in length, more preferably at most 40 nucletoides in length.

Finally, it should be noted that a single pRNA chimera may include more than one type heterologous component. For example, a pRNA chimera may include a folate conjugated to its 5′ end and a detectable label conjugated to its 3′ end. Or, for example, a circularly permuted pRNA chimera may include an RNA aptamer in the spacer region, and a therapeutic siRNA as its paired helical region.

Also included in the invention is a DNA molecule that includes a nucleotide sequence that encodes the pRNA chimera of the invention. The spacer region of the encoded chimera is necessarily RNA in this aspect of the invention. The DNA molecule can be linear or circular. It can be double stranded or single stranded; if single stranded, its complement is included in the term “DNA molecule” as well.

The pRNA chimera of the invention can be introduced into a host cell in a number of different ways. For example, the pRNA chimera can be synthesized outside the cell, contacted with the cell surface such that a constituent RNA aptamer or other targeting agent binds to a component of the cell surface, and taken up by the cell via receptor-mediated endocytosis, membrane diffusion, transport through a pore, or the like. Alternatively, it can be delivered as part of the genetic cargo carried by a viral delivery agent (either an RNA virus or a DNA virus). It can also be delivered as a plasmid, i.e., as a DNA molecule that encodes the desired pRNA chimera. It is also possible to directly transfect the pRNA chimera into the host cell. For example, a product available under the trade designation TRANSMESSENGER TRANSFECTION REAGENT (available from Qiagen), which a lipid-based formulation that is used in conjunction with a specific RNA-condensing enhancer and an optimized buffer, can be used to transfect the pRNA chimera into eukaryotic cells.

A DNA molecule for use in introducing a pRNA into a cell preferably contains regulatory elements such that the pRNA chimera is operably encoded. A pRNA chimera is “operably encoded” by a DNA molecule when the DNA molecule contains regulatory elements that allow the pRNA chimera to be produced by transcription of the DNA molecule inside the cell. Such regulatory elements include at least a promoter. Optionally, the DNA molecule includes additional regulatory motifs that promote transcription of the RNA chimera, such as, but not limited to, an enhancer. The DNA molecule can be introduced into the host cell using anionic or cationic lipid-mediated delivery or other standard transfection mechanisms including electroporation, adsorption, particle bombardment or microinjection, or through the use of a viral or retroviral vector.

Optionally, the DNA molecule can contain one or more features that allow it to integrate into the cell's genome. For example, it can be delivered in the form of a transposon, a retrotransposon, or an integrating vector; alternatively, it can contain sequences that are homologous to genomic sequences that allow it to integrate via homologous recombination. On the other hand, the DNA molecule can be designed to exist within a cell as nongenomic DNA, e.g., as a plasmid, cosmid, episome and the like.

Transcription from a DNA template encoding the entire chimeric RNA molecule can occur in vitro or within a cell. The cell can be in cell culture, or in an organism (in vivo) such as a plant or an animal, especially a human, or in a cell explanted from an organism (ex vivo).

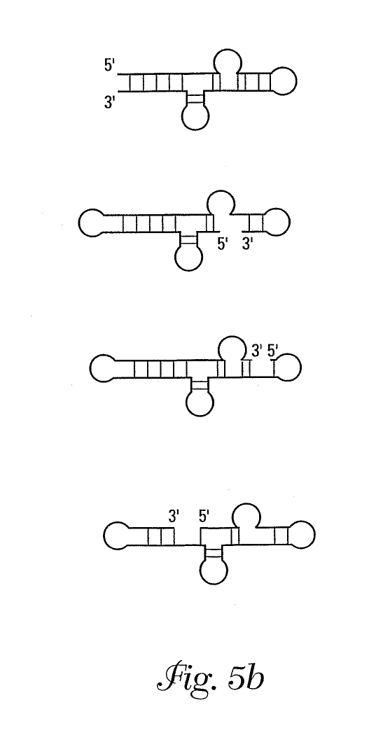

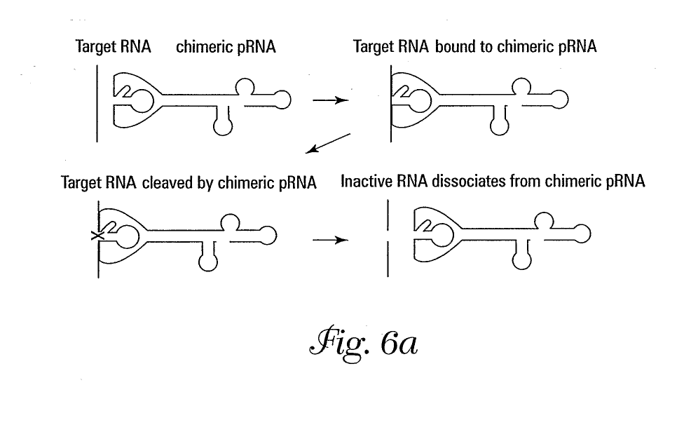

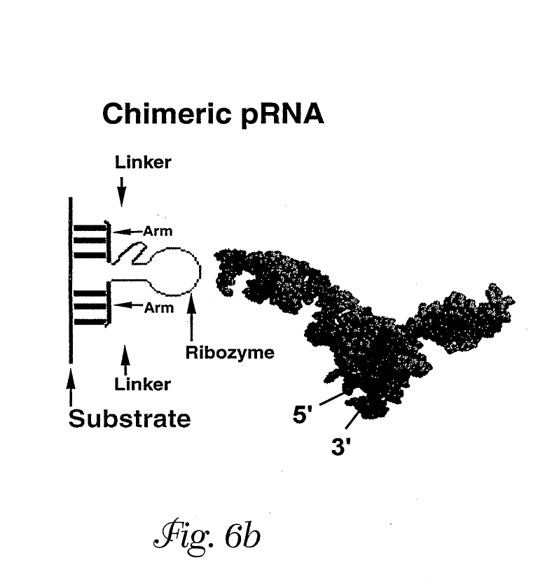

Advantageously, the pRNA chimera of the invention can be used to deliver a biologically active RNA molecule to a target within a cell. A DNA molecule having nucleotide sequence that operably encodes a circularly permuted pRNA region and a spacer region is introduced into a cell. The spacer region includes a biologically active RNA, and transcription of the DNA to yields the biologically active RNA. The biologically active molecule thus delivered is preferably a ribozyme, and the target is preferably viral or mRNA associated with a gene whose expression it is desirable to reduce. FIG. 6(a) shows a proposed mechanism for cleavage of a target RNA by a pRNA ribozyme chimera. The ribozyme targeting the HBV polyA signal is connected to the native 5′/3′ ends of the phi29 pRNA (FIG. 6(b)). An antisense RNA, which can target intracellular DNA or RNA, is also preferred as the biologically active molecule.

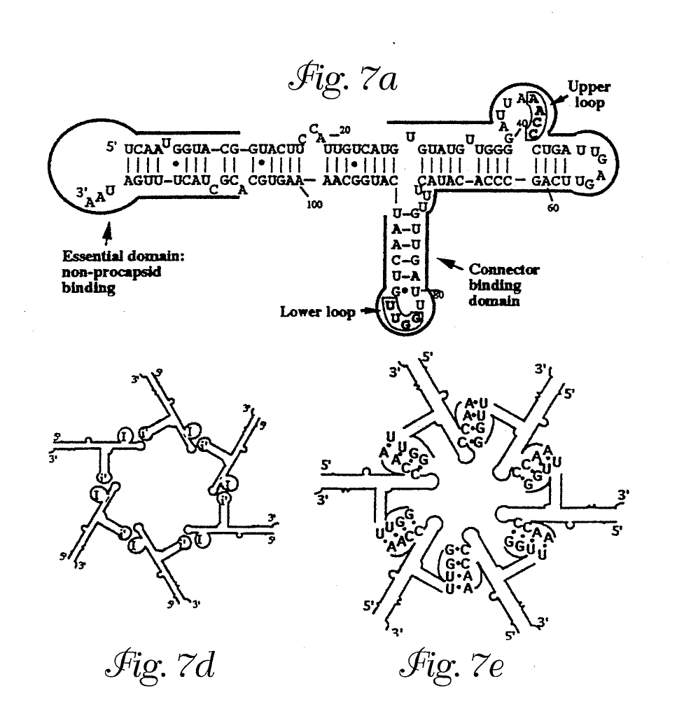

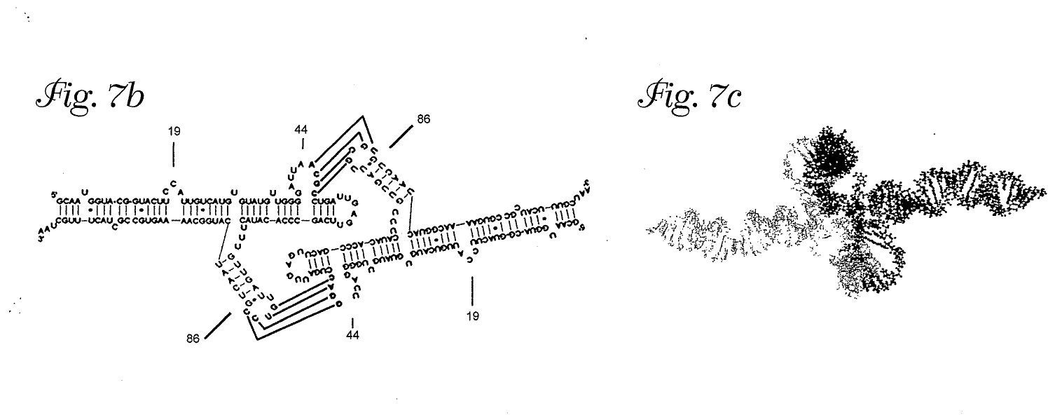

φ29 pRNA has a strong drive to form dimers (FIG. 7), and dimers are the building blocks of hexamers (Hoeprich and Guo, J Biol Chem 277:20794-20803 (2001); Mat-Arip et al., J Biol Chem 276:32575-32584 (2001); Trottier et al., RNA 6:1257-1266 (2000); Chen et al., RNA 5:805-818 (1999); Guo et al., Mol Cell 2:149-155 (1998); Zhang et al., Mol Cell 2:141-147 (1998); Hendrix, Cell 94:147-150 (1998)). The formation of monomers or dimers can be controlled by manipulating and controlling the sequences of the two interacting loops (Hoeprich and Guo., J Biol Chem 277:20794-20803 (2001); Mat-Arip et al., J Biol Chem 276:32575-32584 (2001); Trottier et al., RNA 6:1257-1266 (2000)); Chen et al., RNA 5:805-818 (1999); and Zhang et al., Mol Cell 2:141-147 (1998)).

We have shown previously that six copies of pRNA form a hexameric ring (Guo et al., Mol. Cell., 2:149-155 (1998); Hendrix et al., Cell, 94:147-150 (1998); and Zhang et al., Mol. Cell., 2:141-147 (1998)) to drive the DNA-packaging motor (see Grimes et al., Adv. Virus Res., 58:255-294 (2002) and Guo, Prog in Nucl Acid Res & Mole Biol., 72:415-472 (2002) for a review). pRNA dimers are the building blocks of hexamers (Chen et al., J Biol Chem, 275(23):17510-17516 (2000)). Hand-in-hand interaction of the right and left interlocking loops can be manipulated to produce desired stable dimers and trimers (Chen et al., RNA, 5:805-818 (1999); Guo et al., Mol. Cell., 2:149-155 (1998); Shu et al., J Nanosci and Nanotech (JNN), 4:295-302 (2003); and Zhang et al., Mol. Cell., 2:141-147 (1998)); hexamers are formed via hand-in-hand interaction by base-pairing of two interlocking left- and right-hand loops (Chen et al., RNA, 5:805-818 (1999); Guo et al., Mol. Cell., 2:149-155 (1998); and Zhang et al., Mol. Cell., 2:141-147 (1998)). Thus, pRNA has a strong tendency to form circular rings by hand-in-hand interaction, whether it is in dimer, trimer or hexamer form (Chen et al., RNA, 5:805-818 (1999) and Shu et al., J Nanosci and Nanotech (JNN), 4:295-302 (2003)).

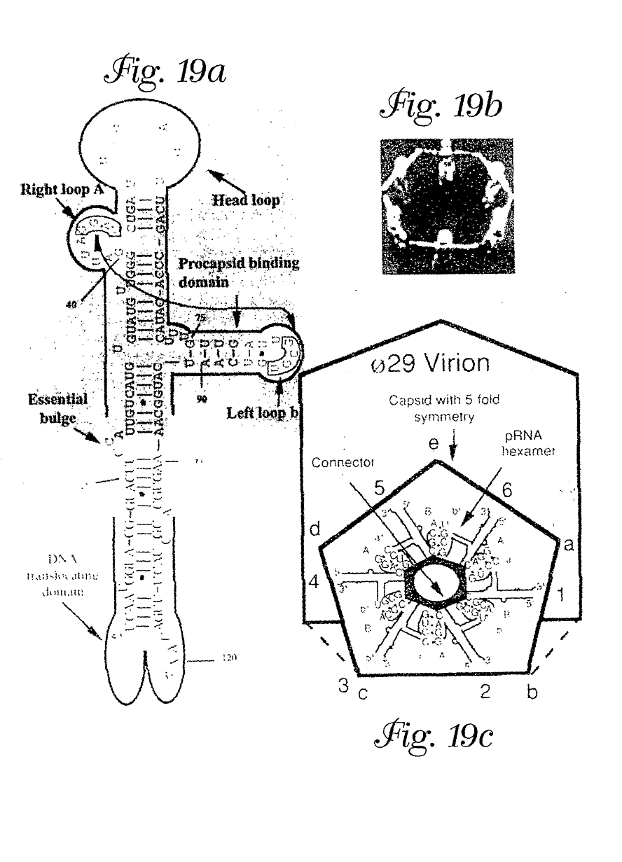

The stoichiometry of pRNA has been investigated by gel, sedimentation (Shu et al., J Nanosci and Nanotech (JNN), 4:295-302 (2003)), binomial distribution (Trottier et al., J. Virol., 71:487-494 (1997)), cryo-AFM(atomic force microscopy), and by mixing two inactive mutant pRNAs with complementary loops intermolecularly, then assaying the activity of the mixtures in DNA packaging assays (Guo et al., Mol. Cell., 2:149-155 (1998) and Zhang et al., Mol. Cell., 2:141-147 (1998)). The predicted secondary structure of the pRNA (FIG. 19) reveals two loops, called left- and right-hand loops (12). The sequences of the two naturally occurring loops (bases 45-48 of right hand loop and bases 85-82 of left-hand loop) are complementary. Chemical modification interference was used to distinguish the bases that are involved in intermolecular associations (i.e., dimer formation) from those which are not involved. Bases 45-49, 52, 54-55, 59-62, 65-66, 68-71, 82-85, and 88-90 showed very strong involvement in dimer formation. Chemical modification interference, chemical probing and cryo-AFM revealed that the dimer was formed via hand-in-hand and head-to-head contacts, an atypical and novel RNA dimerization that is distinct from other reported interactions such as pseudoknots or the kissing loops of HIV (Chang et al., J Mol Biol, 269(1):52-66 (1997); Laughrea et al., Biochemistry, 35(5):1589-1598 (1996); Muriaux et al., J Biol Chem, 271(52):33686-33692 (1996); Paillart et al., Proc Natl Acad Sci U.S.A, 93:5572-5577 (1996); and Puglisi et al., Nature, 331:283 (1988)).

Chemical modification experiments suggests that C¹⁸C¹⁹A²⁰ is present on the surface of the pRNA as a bulge used to interact with other DNA-packaging components (Trottier et al., RNA, 6:1257-1266 (2000)). Chemical modification also revealed unpaired bases in loops and bulges such as bases 18-20, 42-48, 55-57, 82-86 as well as single-base bulges A⁹, C¹⁰, U³⁶, A⁹³ and A¹⁰⁰. And U⁷²U⁷³U⁷⁴ bases exist as a bulge present at the three-way junction in order to provide flexibility in folding and serve as a hinge for the twisting of the left hand stem-loop (Trottier et al., RNA, 6:1257-1266 (2000)). Chemical modification revealed that three of the major loops were strongly modified in monomers but were protected from modification in dimers.

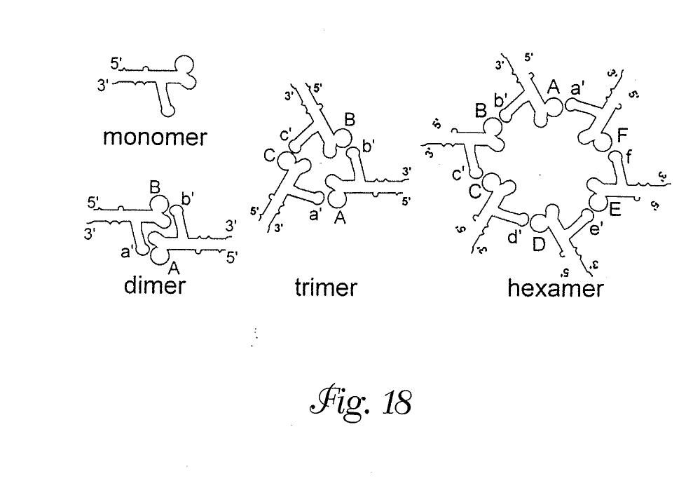

To simplify the description of the subunits in the deliverable complex, we use uppercase letters to represent the right hand loop of the pRNA and lowercase to represent the left hand loop (FIGS. 7, 18). The same letters in upper and lower cases indicate complementary sequences, while different letters indicate non-complementary loops. For example, pRNA A-a′ represents a pRNA with complementary right loop A (^{5′}G₄₅G₄₆A₄₇C₄₈) and left loop ‘a’ (^{3′}C₈₅C₈₄U₈₃G₈₂), while pRNA A-b′ represents a pRNA with unpaired right loop A and unpaired left loop ‘b’ (^{3′}U₈₅G₈₄C₈₃G₈₂). See FIG. 18.

The formation of pRNA dimers (FIG. 7) might also assist in stabilizing pRNA/ribozyme chimera molecules. As long as the openings of the circularly permutated pRNAs are close to an area of dimer formation, the tertiary structure can help prevent exonucleases from accessing the ends of the RNA molecules.

When delivered systemically using prior art methods, the efficiency with which biologically active RNAs such as siRNAs and ribozymes enter the cell is very low due to the large size of the RNA. Currently, most delivery methodologies rely upon transfection and viral vectors. Chemically-mediated transfection procedures can be used in cell cultures but would clearly not be appropriate for delivery to patients. Viral vectors are efficient, but the problems in targeting to specific cells remain to be resolved.

The uptake of extracellular macromolecules and particles by receptor-mediated endocytosis (RME) is a process common to almost all eukaryotic cells. The mechanism for receptor-mediated endocytosis has been subjected to intense scrutiny and its overall feasibility for the delivery of therapeutic molecules, such as antibodies (Becerril et al., Biochem.Biophys.Res. Commun., 255:386-393 (1999) and Poul et al., J Mol. Biol., 301:1149-1161 (2000)), drugs or RNA aptamers (Homann et al., Bioorg. Med Chem, 9:2571-2580 (2001)) has been reported. However, difficulties in exploiting receptor-mediated endocytosis (RME) for the targeting and delivery of therapeutic agents have been encountered and include 1) lack of specificity for the targeted cell versus healthy cells; 2) lysosomal degradation of the therapeutic molecules in the endocytic pathway; 3) instability of the targeting and delivery system in the body, and 4) adverse immunological response associated with repeated doses.

The ability of the pRNA chimera of the invention to form dimers, trimers, tetramers and hexamers opens up exciting new opportunities for diagnosis and treatment of disease. Each individual pRNA chimera can be viewed as a chimeric pRNA “building block”. These building blocks can be designed and selected to serve as components of a customized or “designer” multimeric pRNA complex. The monomeric building blocks are engineered to include right hand and left hand loops in the intermolecular interacting domain that promote the desired interactions (e.g., A-b′, B-e′, E-a′ to form a trimer). See Shu et al., Nano Letters 2004;4:1717-1724; WO2005/035760, published Apr. 21, 2005. The right hand loop includes at least nucleotides 45-48, and the left hand loop includes at least nucleotides 82-85 (see Example 11).

The polyvalent nature of these multimeric complexes makes possible an integrated, multi-faceted approach to treatment and/or diagnosis. As illustrated in Examples 11 and 12 and FIG. 23, there are many permutations of multimeric pRNA complexes that can be envisioned and created using the derivatizations and modifications described herein. A library of pRNA monomers can be designed, and a multimeric complex customized for a particular application can be readily constructed by selecting the desired chimeric pRNA building block from the library. For example, a multimeric complex can be made from a pRNA chimera that includes a therapeutic siRNA in the helical region, a pRNA chimera that includes an RNA aptamer for guidance and targeting, and/or a pRNA chimera that has been derivatized on one or both ends with a detectable label.

The present invention thus offers a mechanism for addressing the difficulties previously encountered in attempts to use receptor-mediated endocytosis for delivery of therapeutic agents. The multimeric nature of pRNA facilitates the construction of a stable, polyvalent pRNA chimera (i.e., a multimeric pRNA complex) according to the invention that carries multiple components for specific cell recognition, endosome escape, and/or delivery of one or more therapeutic molecules.

A dimeric complex, for example, can contain two spacer regions and hence two biologically active moieties. A preferred dimeric complex is one that includes a first chimeric pRNA that includes a targeting moiety (such as an RNA aptamer, and antibody, or a receptor ligand, like folate), and a second chimeric pRNA that includes a biologically active RNA, such as an siRNA, a ribozyme or an antisense RNA.

The strategy of pRNA dimer-mediated delivery is that the receptor-binding moiety mediates cell recognition and subsequent internalization, and the therapeutic RNA, e.g., siRNA, is then released to down-regulate specific genes. The targeting moiety targets the pRNA dimer to the intended cells, and binds to a cell surface receptor. In doing so, it preferably stimulates receptor-mediated endocytosis, thereby causing internalization of the pRNA dimer. When the disease being treated is a viral disease such as infection with HIV or hepatitis virus, the targeting moiety may target specific virus-glycoproteins incorporated on the infected cell surface. The biologically active RNA is an RNA regulates a cell function, and thereby affects cell growth, death, physiology, and the like. Preferably, the biologically active RNA is a therapeutic siRNA or a ribozyme.

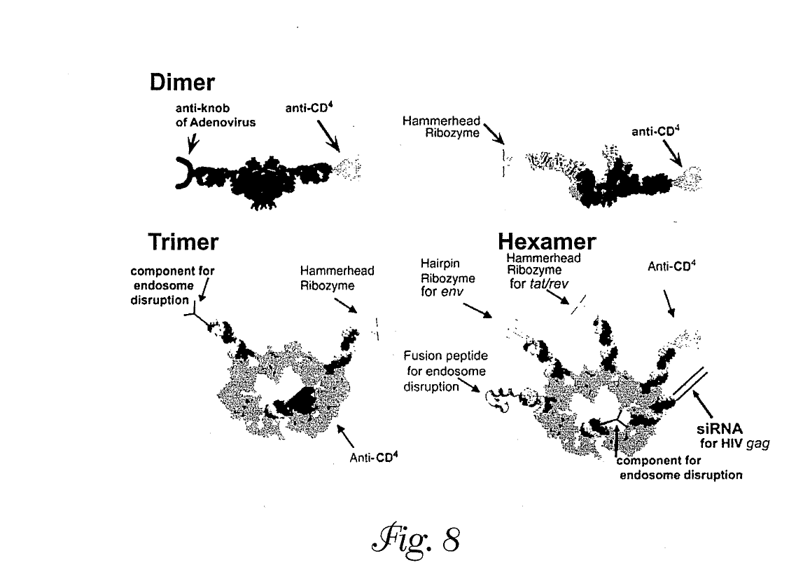

A more complicated hexameric complex is illustrated in FIG. 8. A hexameric complex could include, in addition to a pRNA harboring a targeting moiety, multiple pRNAs harboring different therapeutic agents (e.g., one that contains an siRNA, another that contains a ribozyme). Additionally, a hexamer could include a pRNA chimera that carries a labeling agent, such as a heavy metal, quantum dot, fluorescent dye or bead, or radioisotope. Likewise, a hexamer could include a one or more pRNA chimera that carry a component, such as a fusion peptide, capable of enhancing endosome disruption so that the therapeutic molecules are released. The hexamer could also include a pRNA chimera designed to allow for the detection of apoptosis.

In a preferred embodiment, one subunit of the polyvalent pRNA complex carries a targeting agent, preferably an RNA aptamer, such as a CD4 aptamer (described in more detail below), an antibody, or a ligand that binds cell surface receptor, thereby inducing receptor-mediated endocytosis. The targeting moiety could also interact with some component of the cell membrane or cell wall, and gain entry into the cell by a mechanism other than receptor-mediated endocytosis. As noted previously, targeting moieties, like other “cargo” (heterologous components) that can be carried by the pRNA chimera, can be included in the spacer region of a circularly permuted pRNA chimera (particularly if they are formed from RNA, like an RNA aptamer) or they may be covalently linked to 5′ or 3′ end of a non-circularly permuted pRNA. A preferred receptor ligand useful as a targeting moiety is folic acid (folate). Folate binds to the folate receptor, which is often overexpressed in cancer cells (see Example 13). In a preferred embodiment, designed to increase the accessibility of the folate to folate receptor on the cell surface, the chimeric pRNA is designed with a 5′ overhang, and folate is conjugated to the 5′ end of the pRNA. This can be accomplished by adding nucleotides to the 5′ end, or deleting nucleotides from the 3′ end of the pRNA. Thus, a pRNA chimera conjugated to folate is a preferred component of a multimeric pRNA complex designed to treat or detect cancer.

Another one or two subunits of the pRNA complex optionally carry components that enhance endosome disruption for the release of the delivered therapeutic molecules from the endosome. A number of substances that disrupt endosomes and mediate endosome escape of therapeutic molecules are described in the literature. Defective or psoralen-inactivated adenovirus particles have shown promise since they have considerable endosomolytic activity (Cotton et al., Proc Natl Acad Sci USA, 89:6094-6098 (1992)). Synthetic peptides that mimic the membrane-fusing region of the hemaglutinin of influenza virus have also been successfully used in gene delivery systems to facilitate endosomal escape (Mastrobattista et al., J Biol Chem, 277:27135-27143 (2002); Plank et al., J Biol Chem, 269:12918-12924 (1994); and Van Rossenberg et al., J Biol Chem, 277:45803-45810 (2002)). Polymeric endosome disrupting gene delivery vectors, such as poly(amino ester)(n-PAE) (Lim et al., Bioconjug. Chem, 13:952-957 (2002)) or poly (DL-lactide-co-glycolide) (PLGA) (Panyam et al., FASEB J, 16:1217-1226 (2002)) will also be tested. Endosome disrupting agents can be conveniently linked to the polyvalent pRNA complex by including one pRNA chimeric subunit that contains an RNA aptamer (described in more detail below) designed to specifically bind the endosome disrupting agent. The pRNA chimera thus preferably includes, or is co-delivered with, an endosome disrupting agent, Another pRNA chimera can contain a labeling agent, for example, a heavy metal, quantum dot, optically active moieties (such as fluorescence labels), biotin, or radioisotopes. A multimeric pRNA complex containing such a pRNA chimera may be useful for detection of an affected cell, such as a cancer cell.

Another pRNA chimera can include, as a heterologous component, a separate DNA or RNA oligonucleotide annealed to the 5′ or 3′ end of the pRNA. Preferably, in this embodiment, the pRNA chimera includes a DNA oligonucleotide annealed to the 3′ end of the pRNA. Optionally, the DNA oligonucleotide is derivatized; for example it may contain a detectable label, such as a radiolabel, or a biotin moiety, or the like.

Therapeutic agent(s) (e.g., a biologically active RNA such as a ribozyme or a siRNA, or other drug) can be carried by another of the pRNA monomers that make up a dimeric, trimeric or hexameric polyvalent pRNA chimera. Therapeutic agents can include biologically active RNAs, enzymes, chemotherapeutic drugs, and the like. They can be selected to be effective against cancer or infections disease, including those caused by human immunodeficiency virus (HIV) and hepatitis virus, particularly hepatitis B virus (HBV). An example of a therapeutic agent can be incorporated into a pRNA chimera for use as a component of an anti-cancer multimeric complex is siRNA directed against the gene encoding survivin. Survivin inhibits apoptosis in certain cancer cells, thus survivin siRNA, which silences survivin, induces apoptosis of cancer cells, as illustrated in Examples 11 and 14.

The dimeric, trimeric and hexameric polyvalent pRNA complexes of the invention are thus ideally suited for therapeutic RNAs or other chemical drugs for the treatment of cancers, viral infections and genetic diseases. Applications of multiple therapeutic agents are expected to enhance the efficiency of the in vivo therapy.

RNA molecules that bind other molecules (such as cell surface receptor-binding RNA molecules or RNA molecules that bind endosome disrupting agents) can, for example, be identified and isolated through SELEX (Systematic Evolution of Ligands by Exponential Enrichment) (Tuerk et al., Science 249:505-510 (1990); and Ellington et al., Nature 346:818-822 (1990)). Such RNA molecules are known as “RNA aptamers.” Starting with a library containing random RNA sequences, in vitro evolution techniques allow for the selection of the RNA molecules that are able to bind a specific pre-identified substrate, such as a ligand or receptor (Ciesiolka et al., RNA 1:538-550 (1995); Klug and Famulok, Molecular Biology Reports 20:97-107 (1994). Receptor-binding (“anti-receptor”) RNA can be inserted into the pRNA vector to form circularly permuted pRNA as described herein. The chimeric RNA carrying the hammerhead ribozyme and the chimeric RNA carrying the anti-receptor could be mixed to form dimers or higher order structures via inter-RNA loop/loop interaction as reported previously (Chen et al., J Biol Chem 275:17510-17516 (2000); Guo et al,. Mol Cell 2:149-155 (1998); Zhang et al., Mol Cell 2:141-147 (1998); and Hendrix, Cell 94:147-150 (1998)). The use of a polyvalent RNA containing an RNA aptamer as an anti-receptor is expected to yield superior specificity compared to protein anti-receptors.

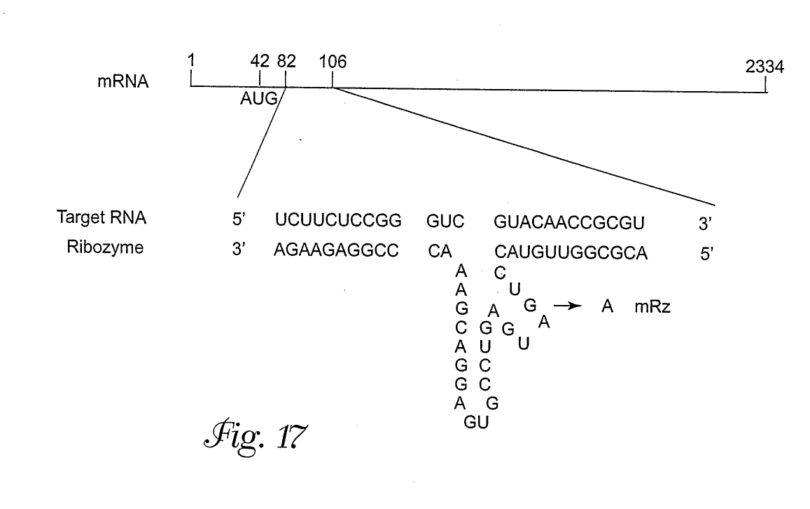

In addition, the basic principles of the SELEX method can be employed to create RNA aptamers using the basic pRNA chimera design of the invention. RNA molecules useful for the identification of RNA aptamers that bind a pre-identified substrate contain a random sequence, preferably 25-100 bases, present at one end of the pRNA of the present invention, preferably connected where the native 5′/3′ ends are located. Optionally, linker sequences connecting the random sequence connected to both ends of the 5′/3′ ends can be included. Such RNA molecules may be made by chemical or enzymatic synthesis. For instance, RNA molecules useful for the identification of RNA aptamers can be made using three primers; a template primer, a 3′ end primer, and a 5′ end primer (see FIG. 17). The DNA primers are designed and defined with reference to a pRNA sequence or its derivatives and counterparts. The template primer includes the random sequence flanked by two nucleotide sequences that bind the 3′ and 5′ end primers. Preferably, each flanking sequence of the DNA template contains a nucleotide sequence having at least 14 bases that are complimentary to the sequences of the 3′ end primer and the 5′ end primer corresponding to the 5′ and 3′ ends of the pRNA.

The 3′ and 5′ end primers can be used to make by PCR the RNA molecules useful for the identification of RNA aptamers, and also for amplification during the SELEX method. The 3′ end primer contains nucleotides that are complementary to an RNA sequence to make a 5′ end of a pRNA sequence, beginning at or about at a 5′ end and ending at any nascent 3′-end, e.g., base 71. Likewise, the 5′ end primer contains the nucleotides that are complementary to an RNA sequence at the 3′ end of a pRNA sequence, beginning at or about at a 3′ end (e.g., around base 117) and ending at any nascent 5′-end, e.g., base 75 (FIG. 17). Taken together, the 5′ and 3′ end primers contain nucleotide sequences complementary to all or most of a pRNA sequence, preferably the wild-type pRNA sequence, such that after transcription the resultant RNA aptamer structure is that of a pRNA chimera of the invention. For example, if the 3′ end primer terminates at base 71 of the wild-type pRNA, and the 5′ end primer terminates at base 75 of the wild-type pRNA, only pRNA bases 72-74 will be missing from the pRNA chimera produced in the SELEX process and this will not affect the independent folding of the pRNA. The secondary structure of the resultant pRNA chimera is equivalent to the phi29 pRNA structure (see FIG. 3 for examples of equivalent structures). For example, the sequence of the 5′/3′ helical region of the pRNA can vary, as long as it forms a paired double stranded region.

The RNA aptamer molecule resulting from this system, which binds the pre-identified substrate, will contain a newly selected RNA sequence connected to the original 5′ and 3′ end of the cp-pRNA, and will be ready for use in a variety of applications without further modification. Such RNA aptamer containing pRNA moiety will be able to bind a pre-identified substrate in variety of applications, including, but not limiting to, drug or gene delivery, and construction of nanodevices.

The SELEX system is used to identify RNA aptamers that bind specifically to proteins, polysaccharides, lipids, ATP, chemicals and theoretically any substance that has a well defined molecular structure (Bouvet, Methods Mol. Biol, 148:603-610 (2001); Ciesiolka et al., RNA, 1:538-550 (1995); Davis et al., Methods Enzymol., 267:302-314 (1996); Gold, Harvey Lect., 91:47-57 (1995); Kraus et al., J Immunol., 160:5209-5212 (1998); Shu et al., J Biol.Chem., 278(9):7119-7125 (2003); Shultzaberger et al., Nucleic Acids Res., 27:882-887 (1999); Wang et al., Sheng Wu Hua Xue.Yu Sheng Wu Wu Li Xue.Bao.(Shanghai), 30:402-404 (1998); and Zhen et al., Sheng Wu Hua Xue. Yu Sheng Wu Wu Li Xue.Bao. (Shanghai), 34:635-642 (2002)). Indeed, this approach can be generalized well beyond being a means to deliver an endosome disrupting agent or bind a target cell surface receptor, as it provides a way to link essentially any desired molecule (typically, a non-nucleic acid) to the pRNA delivery vehicle once an RNA aptamer that binds it has been identified. The linkage between an RNA aptamer and its target molecule is noncovalent, but cross-linking can, if desired, be achieved in some instances after the initial binding step has taken place.

Alternatively, instead of (or in addition to) using SELEX to identify RNA aptamers for specific binding, functional groups such as biotin, —SH, or —NH₂ can be linked to the end of the pRNA. Once the pRNA has been derivatized, endosome disrupting agents (or other desired molecules, particularly non-nucleic acid molecules) can be linked to the end of pRNA by the streptavidin-biotin interaction or by chemical crosslinking (—SH/maleimide or —NH₂/NHS ester).

The ability, disclosed herein, to design pRNA molecules that assemble to hexamers in a preprogrammed, intentional manner lends unmatched versatility to the process. In addition to an anti-receptor aptamer, for example, the hexamer could harbor up to five other components. These could include poly(amino ester)(n-PAE) (Lim et al., Bioconjug. Chem, 13:952-957 (2002)), synthetic peptides (Mastrobattista et al., J Biol Chem, 277:27135-27143 (2002); Plank et al., J Biol Chem, 269:12918-12924 (1994); and Van Rossenberg et al., J Biol Chem, 277:45803-45810 (2002)), virus-derived particles (Nicklin et al., Circulation, 102:231-237 (2000)) for lysosome escape, adjuvants, drugs or toxins. Using the same principle, dimers or trimers could be utilized. Even the hexamer-bound empty procapsid could prove useful, serving as a nanocapsule to harbor DNA coding specific genes for delivery.

Importantly, RNA is uniquely suitable for use in treating chronic diseases since it has a low or undetectable level of immunogenicity except when complexed with protein. (Goldsby et al. In Immunology, 5th ed.; W. H. Freeman and Company: New York, 2002; pp 57-61; Madaio et al., J. Immunol. 1984; 132:872-876). The monomeric or multimeric pRNA chimera of the invention do not contain protein or peptides, and thus the use of such protein-free nanoparticles to avoid immune response allows for long-term administration in the treatment of chronic diseases.

The phylogenetic analysis of pRNAs from Bacillus subtilis phages SF5, B103, phi29, PZA, M2, NF, and GA1 shown in FIG. 3 shows very low sequence identity and few conserved bases, yet the family of pRNAs appears to have similar predicted secondary structures (Pecenlova et al., Gene 199:157-163 (1997); Chen et al., RNA 5:805-818 (1999); Bailey et al., J Biol Chem 265:22365-22370 (1990)). All seven pRNAs of these phages contain both the right and left hand loops, which form a loop/loop interaction via Watson-Crick base pairing. Complementary sequences within the two loops are found in each of these pRNAs. Therefore, these pRNAs could also be used as vector to carry small therapeutic RNA molecules (FIG. 3).

The results from these ribozyme-mediated suppression experiments could be applied to other cell types, including those of many plant and animal species. Transgenic plants and animals could then be developed for a variety of purposes, if the chimeric ribozyme-pRNA is incorporated into the genome of cells, animals or plants.

Surprisingly, conjugation of a ribozyme to a bifurcated pRNA region such that both ends of the ribozyme are covalently linked to the pRNA region does not render the ribozyme inactive, nor does it appear to interfere with the independent folding of the pRNA region or the ribozyme region. Because tethering of both ends of the ribozyme RNA is expected to also prevent degradation by exonuclease, the resulting pRNA-ribozyme chimera is expected to be useful to cleave undesired RNAs in plants and animals, including humans. Additionally, transgenic plants and animals with resistance to diseases can be developed by introducing DNA encoding the pRNA-ribozyme chimera into the genomic DNA of the cell.

The pRNA chimera of the invention is also useful in vitro, for example, for the characterization of RNA molecules. RNA molecules, particularly small RNA molecules, can be stabilized or “chaperoned” by inclusion in the spacer region of a pRNA chimera of the invention, which insures that they remain properly folded, active and exposed. For example, pRNA chimera containing an RNA of interest can be immobilized, covalently or noncovalently, on a substrate, such that the RNA of interest is presented. The immobilized pRNA chimera can then be contacted with test molecules, such as cellular extracts or components, to identify the constituents to which the RNA of interest binds or otherwise interacts. This is preferable to immobilizing the RNA of interest directly on the substrate, because direct immobilization can interfere with the folding of the RNA of interest and also block portions of the structure from contact with the test molecules. The pRNA chimera can also be used to stabilize RNAs in solution for use in binding assays, cleavage assays, diagnostics and the like.

### Examples

The present invention is illustrated by the following examples. It is to be understood that the particular examples, materials, amounts, and procedures are to be interpreted broadly in accordance with the scope and spirit of the invention as set forth herein.

### Example 1

### Elongation of phi29 pRNA at the 3′ End and Effect on Activity

To investigate whether additional burdens can be imposed on the pRNA, the 3′ ends of the pRNA were extended with variable lengths.

The RNA products Eco-pRNA and XbHi-pRNA were produced by in vitro T7 RNA polymerase transcription using DNA templates from plasmid pCRTMII that were precleaved with EcoRI or XbaI/HindIII, respectively. To generate the plasmid pCRTM2, a PCR DNA fragment was produced with the primer pair P7/P11 to flank the pRNA coding sequence (Zhang et al., Virology 207:442-51 (1995)). The PCR fragment was then cloned into the PCR cloning vector pCRTMII (Invitrogen, Carlsbad, Calif.). DNA sequencing after colony isolation confirmed the presence of the PCR fragment in the plasmid. The RNA product 174-pRNA was either extracted from procapsids, as described by Guo et al. (Science 236:690-94 (1987)) and Wichitwechkam et al. (Nucl. Acids Res. 17:3459-68 (1989)) or transcribed in vitro with a PCR DNA fragment generated using the plasmid pCl3-12A(RNA) as template, following the method described in Wichitwechkarn et al. (Mol Biol 223:991-98 (1992)). The RNA product Di-RNA with a 120-base extension from the 3′-end of pRNA was transcribed in vitro with a PCR DNA fragment using cpDNAT7, as described by Zhang et al. (Virology 207:442-51 (1995)) as template for a PCR reaction.

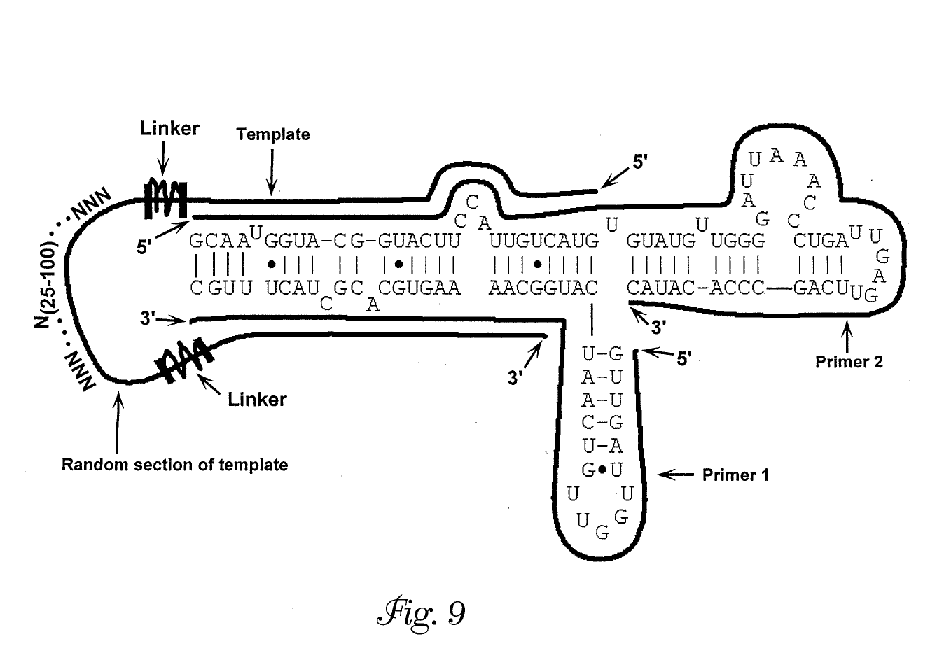

It was found that at least 120 bases could be added to the 3′ end of the pRNA without significant interference of pRNA function (FIG. 9). Such additions included end labeling of pRNA with biotin, pCp, DIG and phosphate. Variable lengths of sequences added to the 3′ end of pRNA had undetectable or minimal impact on viral activity. These results indicated that the 117-base pRNA folded independent of bases extended from its 3′-end.

### Example 2

### Circularly Permuted φ29 pRNA

Circularly permuted pRNA (cpRNA) from bacteriophage φ29 was synthesized by way of transcription form a DNA template. The feasibility of constructing circularly permuted RNAs lies in the close proximity of the native φ29 RNA 5′ and 3′ ends (Zhang et al., Virology 201:77-85 (1994)). φ29 pRNA 5′ and 3′ ends are in close proximity. Construction of biologically active circularly permuted pRNAs revealed that interruption of pRNA internal bases did not affect the global folding of pRNA.

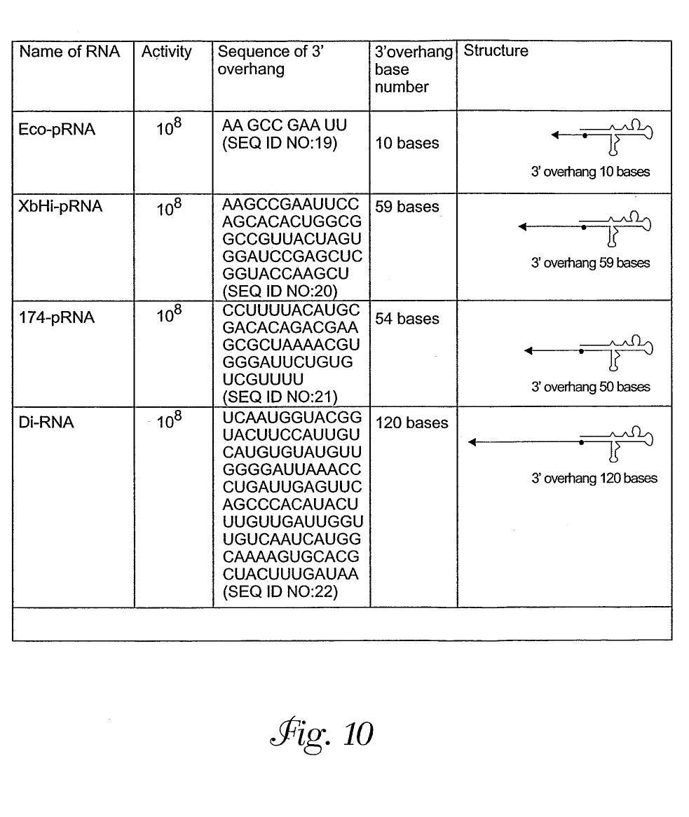

To construct circularly permuted pRNA, two tandem pRNA-coding sequences separated by a 3-base or 17-base loop sequence were cloned into a plasmid (FIG. 10) (see, Zhang et al., Virology 207:442-451 (1995). Plasmids cpDNA3A (I) and cpDNAT7 (II) containing a tandem pRNA coding sequence were connected by 3- or 17-nucleotide synthetic loops, respectively. PCR was used to create dsDNA fragments with non-native 5′/3′ ends. In vitro transcription was then performed to generate pRNAs with new 5′/3′ ends. PCR primer pairs, such as P6/P5, complementary to various locations within pRNA coding sequences, were designed to synthesize PCR fragments for the transcription of cp-pRNAs. The PCR DNA fragments were directly used as templates for in vitro transcription with SP₆ RNA polymerase. The resulting linear cpRNA transcript linked the native 5′-end of pRNA with its 3′ end by way of small loop: AAA in the case of DNA template cpDNA3A and TAATACGACTCACTATA (SEQ ID NO:8) in the case of DNA template cpDNAT₇.

FIG. 11 shows generalized circularly permuted pRNA structure (SEQ ID NO:2) with arrows indicating various new openings (Zhang et al., RNA 3:315-323 (1997)). Wild-type sequences of 5′U1C2 and 3′A117G116 could be changed to G1G2 and C116C117, respectively, relative to wild-type pRNA to facilitate and enhance transcription by T7 RNA polymerase.

To our surprise we found that insertion of sequences to link the native 5′ and 3′ ends of the pRNA molecule and relocation of the 5′ and 3′ ends somewhere else on the molecule does not interfere with the pRNA activity, since the cpRNA was still able to catalyze φ29 assembly. Therefore, most of the internal bases could be used as new termini for constructing active cp-pRNA (Zhang et al., Virology 207:442-451 (1995); Zhang et al., RNA 3:315-322 (1997)).

Since linking the 3′ and 5′ ends of the pRNA with nucleotide sequences of variable lengths did not affect the pRNA activity, this is an indication that pRNA and the linking sequence fold independently. These findings imply that a ribozyme could be placed between the 3′ and 5′ ends of the pRNA could be able to fold without being influenced by the sequence and folding of pRNA.

### Example 3

### In Vitro Activity of pRNA-Ribozyme Chimera

The loop used to connect the native termini of the pRNA in Example 2 did not itself possess any biological activity. However, we wondered whether an RNA sequence with biological activity would retain its activity if tethered at both ends to pRNA. It was decided to test a hammerhead ribozyme as the loop sequence.

An in vitro model system (FIG. 12) as previously described in Cotton et al. (EMBO J. 8:3861-3866 (1989)) was modified and used as a control to test the functionality of a pRNA-ribozyme chimera. U7snRNA (SEQ ID NO:4) was selected as the target RNA. A chimeric RNA molecule, pRNA-RzU7 (SEQ ID NO:3), was synthesized. This system was used to determine whether the pRNA could harbor other hammerhead ribozymes to function in substrate cleavage (Cotten and Birnstiel, EMBO J 8:3861-3866 (1989)).

RNAs were prepared as described previously by Zhang et al. (Virology 201:77-85 (1994)). Briefly, DNA oligonucleotides were synthesized with the desired sequences and used to produce double-stranded DNA by PCR. The DNA products containing the T7 promoter were cloned into plasmids or used as substrate for direct in vitro transcription. The anti-sense DNA encoding the U7 substrate and the DNA encoding ribozyme RzU7 were mixed with the T7 sense promoter prior to transcription. The dsDNA encoding ribozyme RzU7-pRNA and T7 promoter were made by PCR. RNA was synthesized with T7 RNA polymerase by run-off transcription and purified from polyacrylamide gels. Sequences of the plasmids and PCR products were confirmed by DNA sequencing.

The relative abilities of the U7-targeting ribozyme (47 bases), RzU7, and the U7-targeting pRNA-ribozyme (168 bases), RzU7-pRNA, to cleave an U7snRNA fragment were compared. The ribozyme cleavage reaction was done as a control experiment to demonstrate that ribozyme reactions work correctly without any modifications. The results reveal that the RzU7-pRNA ribozyme was able to cleave the substrate with results comparable to the control RzU7 ribozyme (FIG. 12). Extended investigation revealed that specific hammerhead ribozymes harbored by pRNA, were able to cleave other respective substrates.

The RNAs used in these experiments were generated by T7 polymerase in vitro transcription either using PCR or by cloning into a plasmid. The transcription products are as follows:
T7 transcription of pRNA-RzU7 yields the 168 mer:

(SEQ ID NO: 3)

5′GUUGAUUGGUUGUCAAUCAUGGCAAAAGUGCACGCUACUUUGAAAAAC
AAAUUCUAAAACUGAUGAGUCCGUGAGGACGAAAGCUGUAACACAAAAUC
AAUGGUACGGUACUUCCAUUGUCAUGUGUAUGUUGGGGAUUAAACCCUGA
UUGAGUUCAGCCCACAUACA3′

T7 transcription of U7 template yields the 94 mer:

(SEQ ID NO: 9)

5′GGGAAAGCUUAUAGUGUUACAGCUCUUUUAGAAUUUGUCUAGCAGGUU
UUCUGACUUCGGUCGGAAAACGCCUAACGUUGCAUGCCUGCAGGUC3′

T7 transcription of RzU7 template yields the 47 mer:

(SEQ ID NO: 10)

5′GGCAAAUUCUAAAACUGAUGAGUCCGUGAGGACGAAAGCUGUAACA
C3′.

The abilities of RzU7 (47 bases) (SEQ ID NO:10) and pRNA-RzU7 (168 bases) (SEQ ID NO:3) to cleave U7snRNA (SEQ ID NO:9) were compared. The RzU7 cleavage reaction was done as a control experiment to demonstrate that ribozyme reactions work correctly without any modifications. The cleavage reaction using pRNA-RzU7 was done to confirm that pRNA could be successfully used as a carrier molecule for ribozymes.

The U7-targeting ribozyme RzU7 and the ribozyme RzU7-pRNA cleavage reactions were performed at 37° C. for 90 minutes in the presence of 20 mM Tris pH 7.5, 20 mM MgCl₂, and 150 mM NaCl. Control reactions were performed by substituting water for RNAs. The samples were dialyzed against TE (10 mM Tris, 1 mM EDTA, pH 8.0) for 30 minutes on a Millipore 0.025 μm VS type membrane. 2× loading buffer (8 M urea, TBE, 0.08% bromophenol blue, 0.08% xylene cyanol) was added to the samples prior to loading them on a 15% PAGE/8M urea denaturing gel in TBE (0.09 M Tris-borate, 0.002 M EDTA). The gel was stained with ethidium bromide and visualized using EAGLE EYE II (Stratagene).

FIG. 12(b) shows the successful results of the cleavage reaction. The predicted 69 mer and 25 mer cleavage products can be seen.

This experiment confirmed successfully using pRNA as a carrier molecule for ribozymes. The finding that the hammerhead ribozyme retains activity in the pRNA-RzU7 construct has important implications. Independent folding of pRNA apparently and advantageously allows the ribozyme to fold into the correct structure and perform its function in cleaving target RNA.

Furthermore, since both ends of the ribozyme are connected to pRNA, the linkage is expected to protect the ribozyme from exonuclease digestion in the cell. Thus, the ribozyme will be stable after expression in the transgenic plants or animals, solving a persistent problem that has stood in the way of therapeutic use of ribozymes.

### Example 4

### In Vitro Activity of pRNA-Ribozyme Chimera Against Hepatitis B Virus

Hepatitis is a serious disease that is prevalent in many countries worldwide. Hepatitis B virus (HBV) is one causative agent of this disease. HBV is an RNA virus. The RNA genome of HBV was used as target to test the functionality of a chimera pRNA-ribozyme. This work is important because it provides potential for the treatment of this serious infectious disease.

A pRNA-based vector based on bacteriophage φ29 was designed to carry hammerhead ribozymes that cleave the hepatitis B virus (HBV) polyA signal. This hammerhead ribozyme designed by Feng et al. (Biol. Chem. 382:655-660 (2001)) cleaves a 137-nucleotide HBV-polyA substrate into two fragments of 70 and 67 nucleotides.

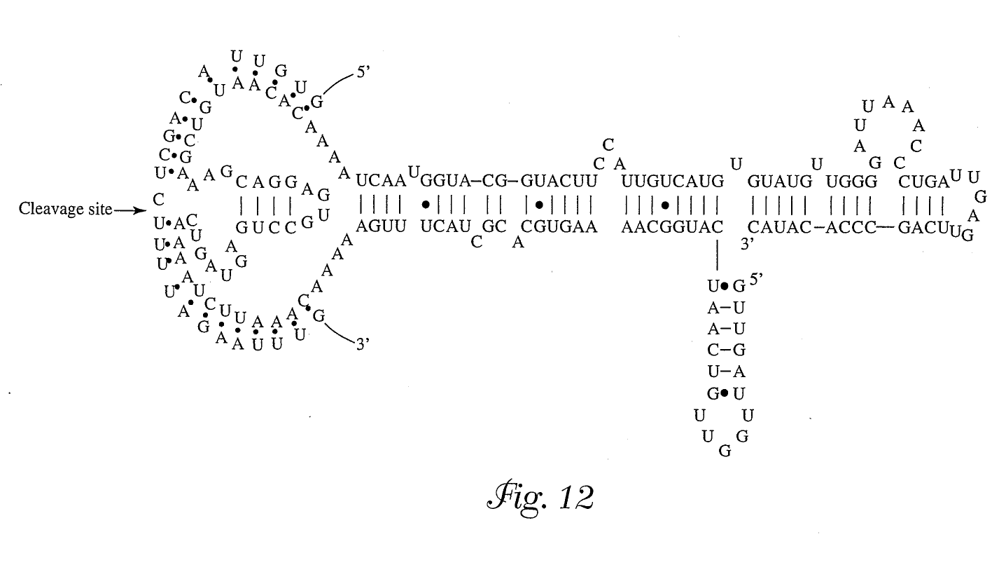

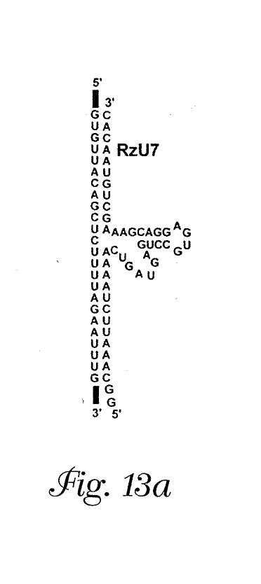

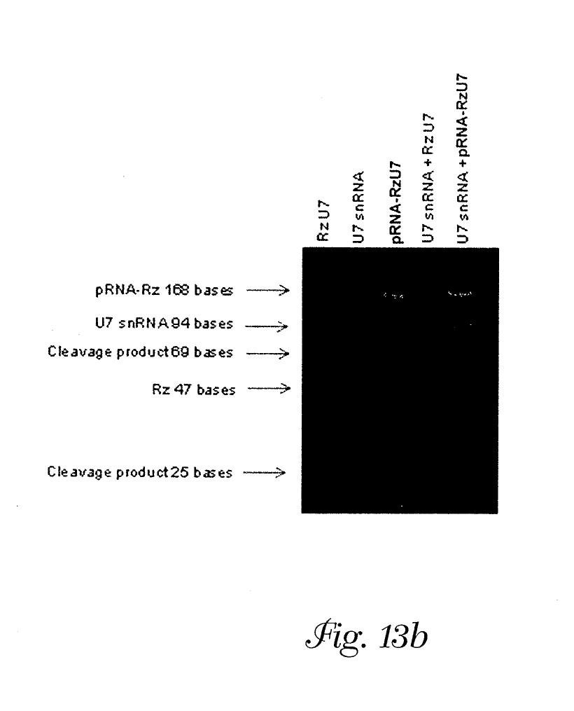

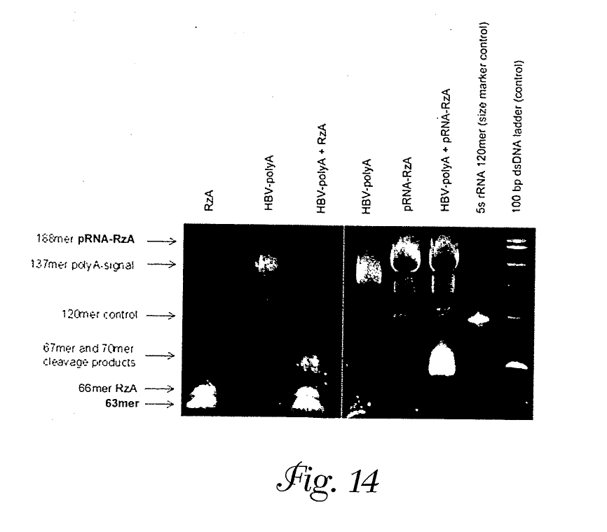

We tested two versions of this ribozyme: pRNA-RzA, which contained a pRNA moiety, and RzA, which did not. The in vitro plasmid pRNA-RzA encoding the chimera ribozyme was constructed by using restriction enzymes XbaI and KpnI to remove the sequence encoding the unmodified ribozyme from the plasmid pRzA, which encoded the ribozyme targeting the HBV polyA signal (Feng et al., Biol Chem 382:655-60 (2001)). Then, a dsDNA fragment made by PCR that encoded the 188 nucleotide chimeric ribozyme was ligated into plasmid pRzA that had been double-digested with Xba I and Kpn I (FIG. 14). The HBV-targeting ribozyme was connected to the 5′ and 3′ ends of pRNA, and the pRNA was reorganized into a circularly permuted form. Two cis-cleaving ribozymes were added to flank the pRNA and HBV-targeting ribozyme.

RNAs were prepared as described previously by Zhang et al. (Virology 201:77-85 (1994)). Briefly, DNA oligonucleotides were synthesized with the desired sequences and used to produce double-stranded DNA by PCR. The DNA products containing the T7 promoter were cloned into plasmids or used as substrate for direct in vitro transcription. The in vitro plasmid pTZS encoding the HBV polyA (Feng et al., Biol Chem 382:655-660(2001)) substrate was linearized with BglII. The in vitro plasmids encoding the HBV polyA substrate targeting ribozyme RzA and the pRNA chimera ribozyme pRNA-RzA were linearized with EcoRI. RNA was produced by in vitro transcription with T7 polymerase using a linear DNA as a template for run-off transcripts. Sequences of the plasmids and PCR products were confirmed by DNA sequencing.

The product of the cis-cleaved transcript, ribozyme pRNA-RzA, was the 188 mer:

(SEQ ID NO: 17)

5′GCUAGUUCUAGAGUUGAUUGGUUGUCAAUCAUGGCAAAAGUGCACGCU
ACUUUGCAAAACAAAUUCUUUACUGAUGAGUCCGUGAGGACGAAACGGGU
CAAAAGCAAUGGUACGGUACUUCCAUUGUCAUGUGUAUGUUGGGGAUUAA
ACCCUGAUUGAGUUCAGCCCACAUACGGUACCUCGACGUC3′

The transcribed ribozyme, RzA, is the 66 mer:

(SEQ ID NO: 18)

5′GCUAGUUCUAGACAAAUUCUUUACUGAUGAGUCCGUGAGGACGAAACG
GGUCGGUACCUCGACGUC3′

The entire cassette of the in vitro plasmid was under the control of a T7 promoter. During transcription of the cassette, the transcript self-cleaved to produce a chimeric ribozyme (pRNA-RzA) containing the HBV-targeting ribozyme that was connected to the pRNA (FIG. 14).

The cleavage reaction was performed at 37° C. for 60 minutes in the presence of 20 mM Tris pH 7.5, and 20 mM MgCl₂. pRNA-RzA (0.539 mmol) was used to cleave HBV-polyA (0.117 mmol). Control reactions were performed by substituting water for certain RNA. The RNA for which water was substituted was omitted from the name of the control. For example, the pRNA-RzA control has no HBV-polyA. The samples were dialyzed against TE (10 mM Tris, 1 mM EDTA, pH 8.0) for 30 minutes on a Millipore 0.025 μm VS type membrane. 2× loading buffer (8 M urea, TBE, 0.08% bromophenol blue, 0.08% xylene cyanol) was added to the samples prior to loading them on a 15% PAGE/8 M urea denaturing gel in TBE (0.09 M Tris-borate, 0.002 M EDTA). The gel was run at 100 volts until the xylene cyanol was 1.5 cm from the bottom of the gel. The gel was stained with ethidium bromide and visualized using EAGLE EYE II by Stratagene.

A dsDNA fragment encoding the pRNA chimera, pRNA-RzA (Table 1), was made by PCR. The pRNA-RzA ribozyme and the HBV-polyA substrate RNA were generated by in vitro transcription with T7 polymerase, using linear DNA as a template for run-off transcripts. This pRNA-RzA ribozyme transcription product then underwent two cis-cleavage reactions to free itself from extraneous RNA flanking sequences. “Cis-cleavage” means a cleavage reaction where both the ribozyme and the substrate are part of the same molecule. These two cis-cleavages were achieved by two ribozymes that flanked the chimera sequence. One cis-ribozyme (63 nt) was 5′ to the chimera, while the other cis-ribozyme (46 nt) was 3′ to the chimera (FIG. 14)). The cis-cleavage reactions predominantly occurred during the time the pRNA-RzA ribozyme was transcribed (FIG. 14).

TABLE 1
Plasmids, oligos and PCR products used
for the assay of ribozyme activities

| | | Pro- | Target or | Contains |
|---|---|---|---|---|
| Name | Function | moter | Purpose | pRNA |
| cpDNA3A | Circularly | SP₆ | Production | Yes |
| (plasmid) | permutated pRNA, | | of cpRNA | |
| | in vitro | | | |
| cpDNAT₇ | Circularly | SP₆ | Construction | Yes |
| (plasmid) | permutated pRNA, | | of chimeric | |
| | in vitro | | ribozyme | |
| pRNA-RzA | Ribozyme, in vitro | T₇ | HBV polyA | Yes |
| (plasmid) | | | | |
| pRzA | Ribozyme, in vitro | T₇ | HBV polyA | No |
| (plasmid) | | | | |
| pTZS | Substrate, in vitro | T₇ | HBV polyA | No |
| (plasmid) | | | | |
| pRNA- | Ribozyme, | CMV | HBV polyA | Yes |
| CRzA | tissue culture | | | |
| (plasmid) | | | | |
| pCRzA | Ribozyme, | CMV | HBV polyA | No |
| (plasmid) | tissue culture | | | |
| pCdRzA | Disabled ribozyme, | CMV | HBV polyA | No |
| (plasmid) | tissue culture | | | |
| P3.6II | HBV genomic RNAs, | | HBV polyA | No |
| (plasmid) | tissue culture | | | |
| U7 | Substrate, in vitro | T₇ | U7 | No |
| (oligos) | | | | |
| RzU7 | Ribozyme, in vitro | T₇ | U7 | No |
| (oligos) | | | | |
| PRNA- | Ribozyme, in vitro | T₇ | U7 | Yes |
| RzU7 | | | | |
| (PCR) | | | | |
| 12-LOX | Substrate, in vitro | T₇ | 12-LOX | No |
| (oligos) | | | | |
| Rz12lox | Ribozyme, in vitro | T₇ | 12-LOX | No |
| (oligos) | | | | |
| PRNA- | Ribozyme, in vitro | T₇ | 12-LOX | Yes |
| Rz12lox | | | | |
| (PCR) | | | | |

The processed product of the cis-cleaved transcript, a 188 mer, was a major band in the gel and was purified. Examination of the gels used to purify pRNA-RzA ribozyme under UV light produced three distinct shadows. The slowly migrating band was the pRNA-RzA ribozyme. The other two bands that migrated much more quickly were the 5′ and 3′-cis cleaving ribozymes. This indicates that the cis-cleavage is complete.

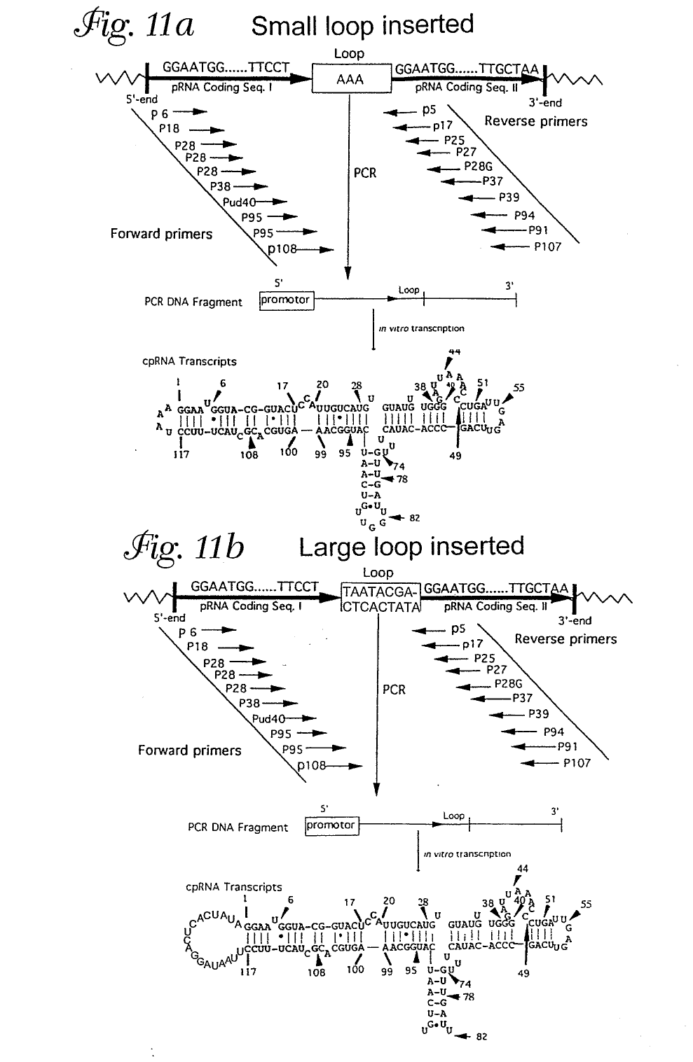

Cleavage of HBV-polyA substrate by the functional chimera pRNA-RzA ribozyme is shown in FIG. 13. The ribozyme pRNA-RzA, which contains a pRNA moiety, was able to cleave the substrate HBV-polyA with nearly 100% efficiency. The predicted 67 base and 70 base cleavage products are seen as one band for the cleavage reaction that included both HBV-polyA and pRNA-RzA ribozyme. The lane labeled pRNA-RzA shows a control reaction that did not contain HBV-polyA, and the lane labeled HBV-polyA shows a control reaction that did not contain pRNA-RzA ribozyme.

The lane labeled RzA in FIG. 13 shows two bands. The upper band (66 nt) is the ribozyme that cleaves the HBV-polyA substrate. The lower band (63 nt) is a cis-cleaving ribozyme produced in the RzA ribozyme transcription reaction. The two ribozymes migrate closely on the gels. The lane labeled RzA-pRNA shows more than one band. The top band is the chimeric ribozyme pRNA-RzA. The lower band is the cleaved products as noted above. No un-cleaved substrate was seen.

In order to use equal molar concentrations of RzA and pRNA-RzA in cleavage reaction, a large mass of pRNA-RzA was used. The other materials shown between the chimeric ribozyme and the cleaved products are degraded chimera ribozyme due to the high RNA concentration in this gel and the large size of the chimeric ribozyme. Even a small percent of degradation resulted in visible degradation products. Due to the secondary structure and incomplete denaturation by urea, the migration rate of RNAs did not match perfectly with the size.

It was found that the hammerhead ribozyme including its two arms for HBV targeting was able to fold correctly while escorted by the pRNA. Comparison of the cleavage efficiency of the ribozyme with and without the pRNA vector revealed a significant difference. The ribozyme pRNA-RzA, which contains a pRNA moiety, was able to cleave the substrate HBV-polyA with nearly 100% efficiency. The chimeric ribozyme cleaved the polyA signal of HBV mRNA in vitro almost completely. However, the ribozyme RzA without the pRNA moiety cleaved the substrate with an efficiency much lower than 70% (not shown).

### Example 5

### Activity of pRNA-Ribozyme Chimera Against Hepatitis B Virus in Cell Culture

A plasmid pCRzA was obtained from Professor Guorong Qi in Shanghai. This plasmid contains sequences coding for a cis-acting hammerhead ribozyme flanked by two sequences targeting hepatitis B virus polyA signal. When this plasmid was co-transfected into HepG2 cells with the HBV genome, HBV RNA level was decreased, and hepatitis B virus replication was inhibited in a dose dependant fashion.

We constructed a plasmid pRNA-CRzA substantially in accordance with Example 3. In pRNA-CRzA, the hammerhead ribozyme and its flanking sequence were carried by the phi29 pRNA, generating a pRNA chimera.

The design of the pRNA-CRzA plasmid used for cell culture studies was basically the same as the one used for in vitro, except that the CMV promoter was used instead of the T7 promoter that was used for the in vitro studies (Table 1). Two versions of this ribozyme were tested: pRNA-RzA ribozyme, which contained a pRNA moiety, and RzA ribozyme, which did not. Both plasmids contain sequences coding for a hammerhead ribozyme targeting the HBV-polyA signal including the two cis-cleaving hammerhead ribozymes.

The tissue culture plasmid pRNA-CRzA encoding the chimera ribozyme was constructed by using XbaI and KpnI to remove the sequence encoding the unmodified ribozyme from the plasmid pCRzA that encoded the ribozyme targeting the HBV polyA signal (Feng et al., Biol Chem 382:655-60 (2001)). Then, a dsDNA fragment made by PCR that encoded the 188 nt chimeric ribozyme was ligated into the position of the plasmid pCRzA that had been double-digested with XbaI and KpnI (FIG. 14).

The HepG2 cells were maintained in Dulbecco's modified Eagle's medium (DMEM) supplemented with 10% fetal calf serum and antibiotics at 37° C. and 10% CO₂. Transient transfection was carried out with the method of calcium phosphate precipitation. In general, cells in 60-mm dishes were transient transfected with 1 μg of HBV expression plasmid p3.6II (Feng et al., Biol Chem 382:655-660 (2001)) and 5 μg of expression construct (CMV vector, pCRzA plasmid (Feng et al., Biol Chem 382:655-660 (2001)) or pRNA-RzA plasmid). 1 μg of pcDNA4LacZ carrying lacZ gene (Invitrogen) was also included in each transfection as internal control. β-galactosidase activity was detected to normalize the transfection efficiency among different dishes.

To analyze HBV viral RNA transcription, seventy-two hours after transfection, the cells were harvested and lysed in TRIZOL reagents (Gibcol-BRL) for total RNA extraction. For northern blot, 20 μg of denatured RNA was resolved in a 0.6M formaldehyde-1% agarose gel and transferred onto HYBOND N+ nylon membrane (Amersham). Probes were prepared by random priming with the 1.8 kb XbaI fragment of HBV (adr) from plasmid p3.6 II and [α-32P] dATP according to the supplier (Promega, Madison, Wis.). After hybridization with HBV probe, the blot was stripped and re-hybridized with a probe of GAPDH (glyceraldehyde-3-phosphate dehydrogenase) that served as an internal control for normalizing the level of total cell RNA.

To analyze e-antigen, seventy-two hours after transfection, cells were harvested and lysed in a buffer (1% NP-40, 50 mM Tris-HCl and 1 mM EDTA) at 37° C. for 10 minutes. Activity of β-galactosidase in cell lysate was determined to normalize the variation of transfection efficiency among different samples. The e-Ag in cell lysates and media was assayed with a commercial ELISA kit (Sino-American Co.) and normalized against β-galactosidase activity. The CMV vector, pCRzA, pRNA-RzA, and disabled ribozyme plasmid pCdRzA were transformed into HepG2 cells together with HBV expressing plamid p3.6II and the β-galactosidae expressing plasmid pcDNA4LacZ serving as an internal control. See Table 2. The amount of CMV vector was arbitrarily taken as 1.

The e-antigen assay was performed to investigate whether the pRNA could enhance the inhibition of HBV replication by hanmmerhead ribozyme. The e-Ag is expressed by translation from a start site upstream of the pre-core (pre-c) coding region, having a nearly identical amino acid sequence as the core antigen, while possessing different antigenicity due to the difference in location of protein expression. The e-Ag appears early during acute HBV

TABLE 2
Comparison of the e-antigen (e-Ag) level of HBV in medium and
cytoplasm of HepG2 cells transfected with different plasmids.

| | e-Ag in media | | e-Ag in cell lysate | | Number of |
|---|---|---|---|---|---|
| | X̅ | | X̅ | | experiments |
| Plasmids | (Normalized) | S.D. | (Normalized) | S.D. | (n) |
| Vector | 1 | — | 1 | — | 3 |
| CrzA | 0.790 | 0.072 | 0.816 | 0.176 | 3 |
| pRNA-RzA | 0.503 | 0.055 | 0.563 | 0.153 | 3 |
| CdRzA | 0.830 | 0.052 | 0.760 | 0.052 | 3 |

infection and is suitable for antigen assay in cell culture.

Assay of e-Ag revealed that pRNA enhanced the inhibition effect of ribozyme by comparing the e-Ag level of cells transfected with plasmids pcRzA (expressing hammerhead ribozyme only), pRNA-RzA (expressing the chimeric ribozyme with pRNA vector), pCdRzA (expressing the disabled ribozyme), and vector only (Table 2). The inhibition by the catalytically inactive ribozyme may be due to an antisense mechanism that involves the hybridization of arm I and arm II to the complementary HBV sequences.

To evaluate the effect of pRNA-RzA ribozyme in cell cultures, ribozyme-expressing plasmids pCRzA, pRNA-RzA, pCdRzA or empty vector was co-transfected with HBV genome-expressing plasmid p3.6 II into hepatoma HepG2 cells. The p3.6II contains 1.2 copies of HBV (adr) genome and produces all viral RNA transcripts (3.51(b pre-core and pre-genomic RNA; 2.4Kb Pre-S RNA, 2.1 kb S RNA and 0.8Kb X RNA) in HepG2 cells without any additional factor. Total cellular RNA was extracted seventy-two hours post-transfection. After normalizing against β-galactosidase activity as an internal control, comparable amounts of RNA (the amount of RNA sample loaded in each lane can be evaluated by GAPDH level) were applied to gel and detected by Northern blotting with an HBV-specific DNA probe. The probe was used to detect the 3.5 Kb and 2.1/2.4 Kb viral RNA as indicated. The presence of pRNA-RzA ribozyme caused an obvious decrease in both 3.5 and 2.1/2.4 Kb HBV RNA level.

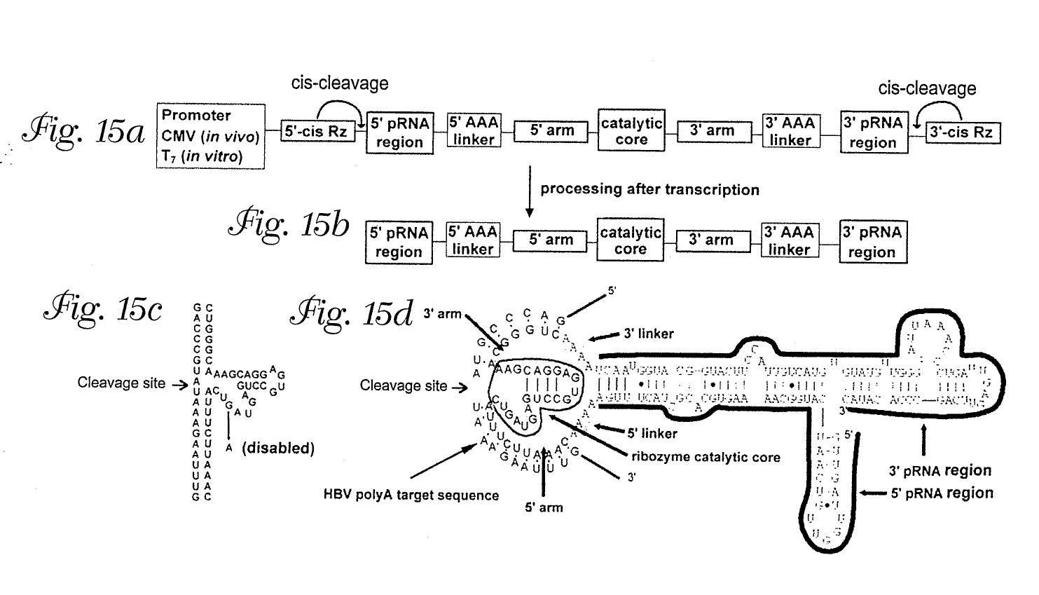

The inhibition by this modified ribozyme was more significant compared with the CRzA ribozyme especially in affecting 2.1/2.4 Kb viral RNA level. The disabled ribozyme CdRzA (encoded by plasmid pCdRzA) bearing one base mutation in Helix II was also used in parallel with CRzA ribozyme and pRNA-RzA ribozyme (FIG. 15).

Antigen assays and Northern blot have demonstrated that phi29 pRNA can chaperone and escort the hammerhead ribozyme to function in the cell, enhancing the cleavage efficiency and inhibition effect of the ribozyme on HBV. The mechanism for such increase in ribozyme activity is probably due to the fact that the pRNA can prevent the ribozyme from misfolding and protect the ribozyme from degradation by exonucleases present in cells. The pRNA molecule contains two independently functional domains: the procapsid binding domain and the DNA-translocation domain (FIG. 2(a)). It was demonstrated that exogenous RNA can be connected to the end of the pRNA without affecting pRNA folding. At least 120 nonspecific bases were extended from the 3′ end of aptRNA without hindering the folding or function of the pRNA, indicating that the 117-base pRNA was folded independent of bases extended from its 3′-end. In addition, construction of biologically active circularly permuted pRNAs revealed that interruption of pRNA internal bases did not affect the global folding of the pRNA. The demonstration that the linking of the 3′ and 5′ ends of pRNA with variable lengths of nucleotide sequence, which did not affect the pRNA activity, is an indication that pRNA and the linking sequence fold independently.

These cell culture studies showed that the chimeric ribozyme was able to enhance the inhibition of HBV replication when compared with the ribozyme not escorted by pRNA, as demonstrated by Northern blot and e-antigen assays. pRNA could also carry another hammerhead ribozyme to cleave other RNA substrate. These studies show that a ribozyme could be placed between the 3′ and 5′ ends of the pRNA and will be able to fold without being influenced by the original pRNA sequence. These findings suggest that pRNA can be used as a vector for imparting stability to ribozymes, antisense, and other therapeutic RNA molecules in intracellular environments.

### Example 6

### Activity of pRNA-Ribozyme Chimera Against Cancer in Cell Culture

Growth and metastasis of solid tumors requires persistent angiogenesis. Angiogenesis is a important process by which new blood vessels are formed. The protein type 12 lipoxygenase (12-LOX) in platelets makes 12-HETE (12-hydroxy-5,8,10,14-eicosatetraenoic acid) by adding O₂ to C-12 arachidonic acid. 12-LOX and its metabolites may be important factors in tumor angiogenesis. The application of this research could restrict tumor growth by preventing cancer cells from prompting blood vessels to grow in the surrounding tissue.

In vitro studies by Liu et al. have shown that this ribozyme, 121oxRz, efficiently cleaved the substrate (Cancer Gene Ther. 7:671-675 (2000)). Efficiency was increased when changing the reaction temperature from 37° C. 20 to 50° C. Studies in cell culture showed that cells expressing the ribozyme from a plasmid had such a decreased level of 12-LOX mRNA that it was undetectable by Northern blotting. A control group of cells that only had a nonfunctional mutant ribozyme had only a slight decrease in the level of 12-LOX mRNA. This slight reduction in 12-LOX mRNA expression could have been the result of an antisense effect by the mutant ribozyme by merely binding to the 12-LOX mRNA without cleaving it. Cell extract was assayed for 12-LOX enzyme activity. Cells expressing ribozymes had 13% of 12-LOX enzyme activity after 6 months compared to parental cells. Cells expressing the mutant nonfunctional ribozyme had 80% of 12-LOX enzyme activity compared to parental cells (Liu et al., Cancer Gene Ther., 7:671-675, 2000). This demonstrates the activity of the ribozyme.

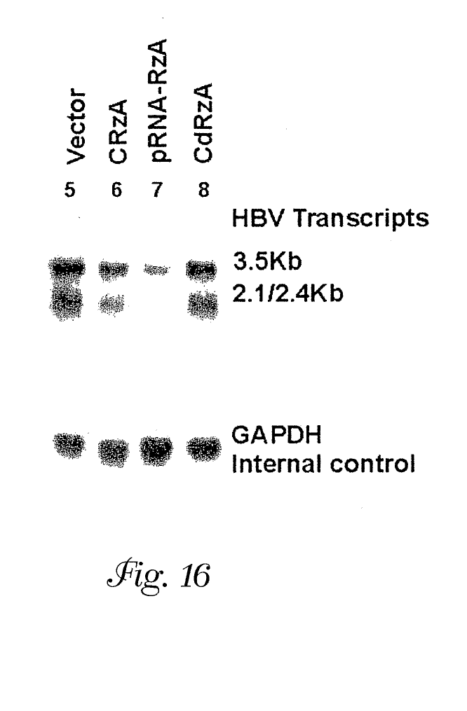

Platelet-type 12-lipoxygenase (12-lox) mRNA (FIG. 16) was selected as a target to test whether a chimera hammerhead ribozyme can function to suppress mRNA levels in human erythroleukemia (HEL) cells. We obtained the in vitro and tissue culture plasmids that encode the ribozyme from Professor Tien, Director of the National Key Lab of Virus Research in which the inventor Peixuan Guo is the Advisor and Visiting Professor. The hammerhead ribozyme was inserted into our pRNA essentially using the method described in Example 3. We created the chimerical ribozyme, 12loxRzpRNA, first constructing a dsDNA template in a two step PCR reaction from oligonucleotides encoding the T7 promoter and the 12loxRz inserted into the pRNA sequence. This template was subsequently transcribed to give the 12loxRzpRNA.

Experiments to test the activity of 12loxRzpRNA will be performed. For the in vitro experiments, the 12loxRz and a target RNA fragment of the 12-lox mRNA (the mRNA substrate) are produced from oligonucleotides essentially using the method described in Example 2. The 12loxRz and the substrate RNA are each transcribed from their own set of two hybridized DNA oligonucleotides. One encodes the negative sense T7 polymerase promoter and the substrate sequence or the 12loxRz sequence. The other oligonucleotide encodes the positive sense T7 promoter sequence. The RNA substrate is radio-labeled using calf intestine phosphatase (CIP) and then polynucleotide kinase (PNK) with [γ³²P]-ATP.

The cleavage efficiency of two ribozymes with and without the pRNA moiety will be evaluated both in vitro and cells (cell culture). For the in vitro study, we will compare the stability of the ribozymes resistance to pH, ion concentration, RNase and cell lysate. These are factors that affect the ribozyme stability and function in the cell.

HEL cells expressing 12-lox will be used for the cell culture experiments. An empty expression cassette or the 12loxRzpRNA in an expression cassette encoding the tRNA^{val} promoter, the 12loxRzpRNA chimera, and the eukaryote polymerase III terminator sequence (5 T residues) will be delivered by transfection using electroporation. Expression of the 12loxRzpRNA chimera and 12-lox mRNA in the cells will be detected by northern blot. Nontransfected HEL cells will be used as a control. 12-LOX enzyme activity will be evaluated by the determination of whether there is a reduction in 12-HETE production in HEL cells.

For both the in vitro and cell culture experiments, a mutant 12loxRz and a mutant 12loxRzpRNA chimera control will be used as a second control. The mutant 12loxRz has one of its nucleotides in its conserved catalytic core domain substituted with another base, rendering the ribozyme unable to cleave the substrate RNA. The use of the non-catalytic mutant ribozymes as a second control is designed to reveal whether the native ribozyme is capable of inhibiting translation by binding to the RNA substrate (i.e., an antisense effect), as opposed to cleaving it.

### Example 7

### Construction of Active Dimers, Trimers and Hexamers

Hand-in-hand interactions between the right and left interlocking loops result in the formation of stable dimers, trimers, or hexamers. pRNA has a strong tendency to form a circular ring by hand-in-hand interaction, regardless of whether the pRNA is in its dimer, trimer or hexamer form. The sequence responsible for intermolecular pRNA/pRNA interaction is located between residues 23-97 (Chen et al., RNA, 5:805-818 (1999)). Change or insertion of nucleotides before residue #23 or after residue #97 does not interfere with the formation of dimers, trimers, and hexamers. The ability to form dimers or trimers is also not affected by 5′ or 3′ end truncation before residue #23 and after residue #97.

Our approach is to construct individual chimeric pRNA monomers that can be “mixed and matched” to carry a therapeutic agent, e.g., a “daughter” RNA molecule such as an siRNA or ribozyme, to a specific target cell. Each monomer subunit is a circularly permuted pRNA as described herein and is designed to have specific right or left loops, such as A (Right)-b′(Left), designed so as to facilitate intermolecular interactions to form a multimer. Each pRNA carries a specific “payload” (e.g., a recepter-targeting aptamer, an endosomal lysing agent, or a therapeutic RNA). Mixing of individual circularly permuted chimeric pRNA with appropriate interlocking loops results in the efficient formation of dimer, trimer or hexamer deliverable complex.

Construction of a pRNA Dimer

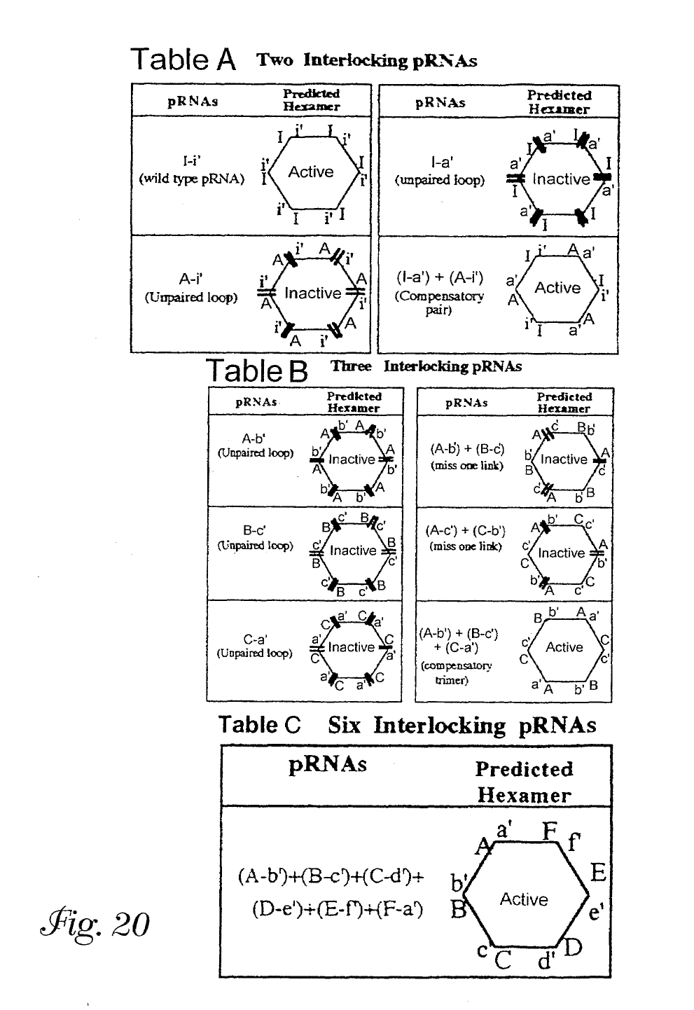

pRNA dimers are formed by intermolecular interaction of the interlocking right and left loops. To simplify the description of the mutants described herein, uppercase and lowercase letters are used to designate the right- and left-hand loop sequences of the pRNA, respectively. The same letter in upper and lower cases symbolizes a pair of complementary sequences. For example, in pRNA A-a′, the right loop A (5′GGAC₄₈) and the left loop a′ (3′CCUG₈₂) are complementary, while in pRNA A-b′, the four bases of right loop A are not complementary to the sequence of left loop b′ (3′UGCG₈₂). Mutant pRNAs with complementary loop sequences (such as pRNA A/a′) are active in phi29 DNA packaging, while mutants with non-complementary loops (such as pRNA A/b′) are inactive (FIG. 20).

We found that pRNAs A-i′ and I-a′ were inactive in DNA packaging alone, but when A-i′ and I-a′ were mixed together, DNA-packaging activity was restored (FIG. 20a; Hoeprich et al., J Biol. Chem., 277(23):20794-20803 (2002)). This result can be explained by the trans-complementarity of pRNA loops, i.e., the right hand loop A of pRNA A-i′ could pair with the left hand loop a′ of pRNA I-a′. Mixing two inactive pRNAs with interlocking loops, such as when pRNA A-b′ and B-a′ are mixed in a 1:1 molar ratio, results in the production of pRNA dimers with up to 100% efficiency. Thus, the stoichiometry of the pRNA is predicted to be a multiple of two (six or twelve).

We constructed several covalently linked dimeric pRNAs that were found to be active in DNA packaging in vitro (Shu et al., J Nanosci and Nanotech (JNN), 4:295-302 (2003)). This further verifies that dimers are the building blocks of the hexamer. Determination of the Hill coefficients of each of these three fully active RNAs implies that for each procapsid, there are three binding sites for dimers and that the binding is cooperative.

Construction of a pRNA Trimer

Another set of mutants is composed of three pRNAs: A-b′, B-c′ and C-a′ (FIG. 20b). This set is expected geometrically to be able to form a 3-, 6-, 9-, or 12-mer ring that carries each of the three mutants. We have constructed several sets of trimers, e.g. A-b′, B-c′ and C-a′. When tested alone, each individual pRNA exhibited little or no activity. When any two of the three mutants are mixed, again little or no activity was detected. However, when all three pRNAs were mixed in a 1:1:1 ratio, DNA packaging activity was restored. Indeed, stable pRNA trimers are formed with very high efficiency, 10 up to 100%, using such sets of three interlocking pRNA (FIGS. 8 and 22). The lack of activity in mixtures of only two mutant pRNAs and the restored activity in mixtures of three mutant pRNAs was expected since the mutations in each RNA were engineered in such a way that only the presence of all three RNAs will produce a closed ring. The fact that the three inactive pRNAs were fully active when mixed together suggests that the number of pRNAs in the DNA-packaging complex was a multiple of 3, in addition to being a multiple of 2 (FIG. 20b). Thus the number of pRNAs required for DNA packaging is a common multiple of 2 and 3, which is 6 (or 12, but this number has been excluded by the approach of binomial distribution and serial dilution analyses that revealed a pRNA stoichiometry of between 5-6) (Trottier et al., J. Virol., 71:487-494 (1997)).

### Construction of a Hexamer

DNA packaging activity is also achieved by mixing six different mutant pRNAs, each of which are being inactive when used alone (FIG. 20c). Thus, an interlocking hexameric ring can be predicted to form by the base pairing of the interlocking loops. The efficiency of formation of pRNA hexamers from dimers in a protein-free solution is low (Guo et al., Mol. Cell., 2:149-155 (1998) and Zhang et al., Mol. Cell., 2:141-147 (1998). However, more than half of the dimer pRNA with appropriate interlocking loops could form hexamers in the presence of an appropriate protein template—the connector or the procapsid (Chen et al., J Biol Chem, 275(23):17510-17516 (2000) and Hoeprich et al., J Biol. Chem., 277(23):20794-20803 (2002)). A hexamer with such a protein template would be useful as a delivery particle since the size of the procapsid particle is only 30 mm×40 mm. The alternative approach would be to make high-yield protein-free hexamer through use of crosslinking agents incorporated into the right or left interlocking loop of the pRNA, as reported in our previous publications (Garver et al., J Biol Chem, 275(4):2817-2824 (2000) and Mat-Arip et al., J Biol Chem, 276:32575-32584 (2001)). These hexamers are generated in the presence of protein, and the protein is removed after crosslinking in order to isolate the hexamer.

### Example 8

### pRNA Molecules Carrying Biologically Active RNA

Chimeric pRNA monomers can be constructed harboring desired “daughter” RNA molecules.

### Hammerhead Ribozyme

Hammerhead ribozymes (Forster et al., Cell, 50:9-16 (1987) and Sarver et al., Science, 247:1222-1225 (1990)) target an RNA substrate sequence by using complementary nucleotides as two arms to base pair to the target RNA. Between the ribozyme's two arms of complementary nucleotides is a short sequence of catalytic RNA that performs cleaving functions against the target RNA. The target site for specific cleavage is the three-base sequence NUH (N=A, C, G, U and H=A, C, U, but not guanosine). The nucleotides on either side of the target sequence should not have a strong secondary or tertiary structure, so that the ribozyme can easily base pair to the target. Methods for the selection of targets for hammerhead ribozyme action have been previously published (Mercatanti et al., J Comput. Biol, 9:641-653 (2002)).

A chimeric pRNA harboring a hammerhead ribozyme that successfully targeted the Hepatitis B virus RNA is described in Example 5. Transcription of the expression cassette resulted in self-cleavage of the transcript, producing a chimeric ribozyme (Example 5). To construct other chimeric pRNA harboring hammerhead ribozymes, the RNA sequence for the ribozyme are likewise connected to the 5′ and 3′ ends of pRNA, and the pRNA is circularly permuted, with the nascent 5′/3-end relocated preferably at residues 71/75 of the original pRNA sequence. The end at 71/75 has been shown to be located in a tightly-folded area (Hoeprich et al., J Biol. Chem., 277(23):20794-20803 (2002)). The chimeric pRNA that harbors the ribozyme contains the appropriate light and left loops for the construction of the dimer, trimer or hexamer complex, as desired. Two cis-acting ribozymes are added to flank the pRNA and ribozyme, as reported in (Hoeprich et al., Gene Therapy, 10(15):1258-1267 (2003)). The entire cassette is preferably under the control of a T₇ promoter for in vitro transcription or a CMV promoter when the cassette is expressed in vivo.

### Hairpin Ribozyme

The hairpin ribozyme (Chowrira et al., Nature, 354:320-322 (1991) and Ojwang et al., Proc Natl Acad Sci USA, 89:10802-10806 (1992)) also targets RNAs by two complementary arms base pairing to the target, but its structure and target sequence requirements are much more restrictive. The sequence requirement of a hairpin ribozyme is BN*GUC, where B is any nucleotide other than adenine. Because a required hairpin of the ribozyme is separated from the rest of the ribozyme by one of the target binding arms, that arm is usually made to be only four nucleotides to keep the ribozyme activity reasonable. But in general, the methods and approach for the construction of chimeric pRNA monomer carrying the specific hairpin ribozyme are similar to those used for the hammerhead ribozyme.

### Antisense RNA

Antisense RNAs are single-stranded RNA molecules complementary to mRNA. It has been shown that antisense RNA can inhibit gene expression in the cell (Coleman et al., Nature, 315:601-603 (1985) and Knecht et al., Science, 236:1081-1086 (1987)). We have previously demonstrated that at least 120 nonspecific bases can be extended from the 3′ end of pRNA without hindering its folding and function (Hoeprich et al., Gene Therapy, 10(15):1258-1267 (2003) and Shu et al., J Nanosci and Nanotech (JNN), 4:295-302 (2003)). Such additions included end labeling of pRNA with biotin, pCp, DIG, SH group and phosphate. Our results indicated that the 117-base pRNA folded independently of bases extended from its 3 ′-end. This finding will apply to the construction of chimeric pRNA monomers carrying antisense RNA that is single-stranded. All antisense RNA used to block gene function is placed at the 3′-end of the pRNA.

siRNA

Recently, post-transcriptional gene-silencing and RNA interference have been investigated extensively in a wide variety of organisms using double-stranded RNAs (McCaffrey et al., Nature, 418:38-39 (2002) and Zilberman et al., Science, 299:716-719 (2003)). This RNA is processed into small interfering double-stranded RNAs (siRNAs) of 19-25 nucleotides (Coburn et al., J. Virol., 76:9225-9231 (2003) and Elbashir et al., Nature, 411:494-498 (2001)), which act as guides for the formation of silencing enzymatic complex required for cleavage of the target mRNAs (Hutvagner et al., Science, 297:2056-2060 (2002) and Volpe et al., Science, 297:1833-1837 (2002)). These siRNAs specifically suppress the expression of a target mRNA with a sequence identical to the siRNA. Although the detailed mechanism of post-transcriptional gene silencing and RNA interference remains to be elucidated, this powerful new technology for selective inhibition of specific gene expression employing siRNAs has shown great promise in the therapy of cancer and viral infections (Carmichael, Nature, 418:379-380 (2002); Li et al., Science, 296:1319-1321 (2002); and Varambally et al., Nature, 419:624-629 (2002)).

We have confirmed that the 5′ and 3′ ends of pRNA are paired to form a double-stranded helix (Hoeprich et al., J. Biol. Chem., 277(23):20794-20803 (2002) and Zhang et al., Virology, 201:77-85 (1994)). This double-stranded region, with more than 30 bases, is an independent domain. Complementary modification studies have revealed that altering the primary sequence does not impact pRNA structure and folding as long as the two strands are paired. We have confirmed that replacement of this double-stranded region with other double-stranded RNA does not hinder the formation of pRNA dimers, trimers and hexamers. This region could therefore be replaced by any double stranded, 19-25-base siRNA. It has been reported recently that hairpin siRNA with a loop to link both ends of the two strands of siRNA could still function in gene silencing (Brummelkamp et al., Science, 296:550-553 (2002); McManus et al., RNA., 8:842-850 (2002); Murchie et al., Mol. Cell, 1:873-881 (1998); Paddison et al., Genes Dev., 16:948-958 (2002); Paul et al., Nat Biotechnol., 20:505-508 (2002); Sui et al., Proc Natl Acad Sci USA, 99:5515-5520 (2002); and Yu et al., Proc Natl Acad Sci USA, 99:6047-6052 (2002)), suggesting that it is possible to connect the siRNA at the end distal to the region for interlocking loops.

### Receptor- or Molecule-Binding Aptamer

In vitro selection of RNA molecules that bind to specific targets has become a powerful tool for the screening of randomized RNA pools to obtain RNA molecules called “aptamers” that specifically bind to target molecules. Starting with a library containing random RNA sequences, in vitro evolution techniques (e.g., SELEX, Systematic Evolution of Ligands by Exponential Enrichment) allow for the selection of RNA molecules that efficiently bind to a specific receptor or ligand with high affinity (Ciesiolka et al., RNA, 1:538-550 (1995) and Klug et al., Molecular Biology Reports, 20:97-107 (1994)). Using this technique, a number of aptamers that specifically recognize many kinds of targets, such as organic compounds, nucleotides, peptides, proteins, and receptors, have been obtained.

Though the SELEX system is powerful, one of its disadvantages is that some of the resultant RNA aptamers bind the substrate with low efficiency. Such a poor result is partially caused by the use of two primers with sequences that are pre-set values rather than being random. We developed a unique system, described herein, for using SELEX to screen RNA aptamers with stable structure and higher affinity for their targets. This system can be used to isolate RNA aptamers that bind to the cell surface receptor with both high specificity and efficiency. Such RNA aptamers are then incorporated into the pRNA via connection to the original 5′/3′ end of the pRNA, and through use of an approach similar to that used for the construction of hammerhead ribozyme escorted by pRNA (Hoeprich et al., Gene Therapy, 10(l5):1258-1267 (2003)). We have successfully constructed chimeric pRNA containing aptamers that bind to CD4 and to gp120 of HIV. Each of these aptamers will be selected for incorporation into a circularly permuted pRNA monomer individually.

### Example 9

### Biotin/Streptavidin Interactions to Form Chimeric pRNA

We have developed procedures to add biotin to either the 5′ or 3′ end of the RNA. For 5′-end RNA labeling, we use a special promoter for T7 RNA polymerase that utilizes biotin-adenosine monophosphate as a substrate for the initiation of RNA transcription (Huang, Nucleic Acids Res, 31:e8 (2003)). For 3′ end labeling, the pRNA complex was annealed with a synthetic biotinylated DNA oligo that is complementary to the 3′ end of the pRNA. In this manner, the following exemplary particles can be incorporated into the deliverable complex: 1) fluorescent streptavidin beads with a size of 50-200 nm, incorporated into the RNA complex by biotin-streptavidin interaction; 2) phi29 procapsid (40 nm) labeled with fluorescence and biotin, then linked to the RNA complex by a streptavidin molecule and purified; 3) biotinylated GFP (green fluorescent protein), linked to the RNA complex by a streptavidin molecule and then purified; and 4) streptavidin nanogold particles with a size of 5-10 nm, incorporated into the RNA complex by biotin-streptavidin interaction. In addition, the RNA can be labeled directly with fluorescence, for example 5′-labeling with Bodipy TMR-C5 (Molecular Probe) (Homann et al., Bioorg. Med Chem, 9:2571-2580 (2001)). Internalization of the chimeric pRNA can be examined by either a fluorescence microscope or a con-focal microscope. Alternatively, the cells can be examined by flow cytometry. For the gold particle, the result is analyzed by electron microscopy.

### Example 10

### Targeting HIV-Infected Cells

CD4 is a receptor displayed on the surface of certain T-helper lymphocytes, and is thus a unique target for the specific delivery of the deliverable RNA complex to the cell. We have constructed chimeric pRNA A-b′ and B-a′ monomers harboring RNA aptamers that bind CD4 (and gp120 as well). These chimeric pRNA with A-b′ loops forms dimer with pRNA B-a′ efficiently.

This chimeric pRNA can be incorporated into one of the subunits of pRNA dimer, trimer or hexamer. The pRNA multimers carrying a CD4-binding RNA aptamer will preferentially enter CD4⁺ cells via interaction with CD4 and endocytosis. Ribozymes or siRNAs that specifically cleave mRNA for cellular CCR5 (Feng et al., Virology, 276:271-278 (2003) and Goila et al., FEBS, 436:233-238 (2003)), or HIV mRNAs for gag, tat (Jackson et al., Biochem Biophys Res Commun, 245:81-84 (2003) and Wyszko et al., International Journal of Biological Macromolecules, 28:373-380 (2003)), rev, env, LTR (Bramlage et al., Nucleic Acids Res., 28:4059-4067 (2003)), or other locations of HIV genomic RNA are fused to other subunits of the pRNA polyvalent complex. Nucleotide derivatives can be incorporated into the pRNA to enhance the stability of RNA by conferring resistance to RNase digestion. These chimeric pRNAs can be evaluated for their efficiency in inhibiting HIV replication in a number of CD4-positive cell lines (FIG. 22). Using fluorescently labeled pRNA harboring a CD4-binding aptamer, we found that this RNA complex binds to the CD4 of a T lymphocyte.

### Example 11

### Controllable Self-Assembly of Nanoparticles for Specific Delivery of Multiple Therapeutic Molecules to Cancer Cells Using RNA Nanotechnology: Use of pRNA/siRNA Chimera

Utilizing RNA nanotechnology, both therapeutic siRNA and receptor-binding RNA aptamer were engineered into individual pRNAs of phi29's motor. The RNA building block harboring the therapeutic molecule was subsequently fabricated into a trimer through the interaction of engineered right and left interlocking RNA loops. The incubation of the free nanoscale particles containing receptor-binding aptamer or other ligands resulted in the binding and co-entry of the trivalent therapeutic particles into cells, subsequently modulating the apoptosis of cancer cells and leukemia model lymphocytes. The use of such antigenicity-free 20-nm particles holds promise for repeated long-term treatment of chronic diseases.

### Introduction

A bacteriophage phi29-encoded small RNA has been shown to play an original and essential role in packaging DNA into procapsids (Guo et al., Science 1987;236: 690-694). This RNA is termed packaging RNA or “pRNA.” pRNA forms dimers, trimers and hexamers with sizes of 20 nm (FIG. 23) via hand-in-hand interaction through the base-pairing of two interlocking left- and right-hand loops (Chen et al., RNA 1999; 5:805-818; Shu et al., J Nanosci and Nanotech (JNN) 2003; 3:295-302; Guo et al., Mol. Cell. 1998; 2:149-155; Zhang et al., Mol. Cell. 1998; 2:141-147).

A computer model of the three-dimensional structure of pRNA building blocks has been constructed (Hoeprich et al., J Biol. Chem. 2002; 277(23):20794-20803) based on experimental data derived from photo-affinity cross-linking (Garver et al., RNA. 1997; 3:1068-1079) and chemical modification interference (Trottier et al., RNA 2000; 6:1257-1266; Mat-Arip et al., J Biol Chem 2001; 276:32575-32584), complementary modification (Zhang et al., RNA 1995; 1:1041-1050; Zhang et al., RNA 1997; 3:315-322) nuclease probing (Reid et al., J Biol Chem 1994, 269, 5157-5162), oligo targeting (Zhang et al., Virology 1995; 211:568-576), competition assays (Trottier et al. J. Virol. 1997; 71:487-494; Trottier et al., J. Virol. 1996; 70:55-61) and cryo-atomic force microscopy (Trottier et al., RNA 2000, 6, 1257-1266; Mat-Arip et al., J Biol Chem 2001, 276, 32575-32584; Chen et al., J Biol Chem 2000; 275(23):17510-17516). pRNA hexamer docking with the connector crystal structure reveals an impressive match with available biochemical, genetic, and physical data concerning the 3D structure of pRNA (Hoeprich et al., J Biol. Chem. 2002;277(23);20794-20803).

Recently, we used the bottom-up assembly characteristics of the building blocks of pRNA to produce a variety of structures and shapes, including rods, triangles, twins, tetramers, and three-dimensional arrays up to several microns in size. (Shu et al., J Nanosci and Nanotech (JNN) 2003; 3:295-302; Shu et al., Nano Letters 2004; 4:1717-1724). Such a process occurs as a result of the interaction of programmed helical regions and loops. Arrays produced by this method have been shown to be resistant to a variety of environmental stresses, including a wide range of salt concentrations, temperatures, and pH levels. (Shu et al., J Nanosci and Nanotech (JNN) 2003; 3:295-302; Shu et al., Nano Letters 2004; 4:1717-1724).

The 117-nucleotide pRNA monomer contains two functional domains: the intermolecular-interacting domain and the double stranded helical DNA-packaging domain (FIG. 23). The intermolecular-interacting domain is located in the central section of the pRNA molecule and contains two interlocking left and right loops that can be engineered for bottom-up assembly, whereas the double-stranded helical DNA-packaging domain is located at the 5′/3′ paired ends. Available data suggests that these two domains fold independently of one another. Structural studies have confirmed that the 5′ and 3′ ends of pRNA are proximate and pair to form a double-stranded helix. (Hoeprich et al., J Biol. Chem. 2002; 277(23):20794-20803; Shu et al., Nano Letters 2004; 4:1717-1724). This double-stranded region (with more than 30 nucleotides) is an independent domain, and the addition or deletion of nucleotides at the 5′ end preceding nucleotide #23 and at the 3′ end following nucleotide #97 does not affect the correct folding of the procapsid-binding domain. Complementary modification studies have revealed that altering the primary sequence of any nucleotide of the helix does not impact pRNA structure and folding if the two strands are paired.

Numerous studies have indicated that siRNA is a double-stranded (ds) RNA helix (Li, et al., Science 2002; 296:1319-1321; Brummelkamp et al., Science 2002; 296:550-553; Jacque et al., Nature 2002; 418:435-438; Carmichael, Nature 2002, 418, 379-380; Elbashir et al., Nature 2001; 411:494-498). To test whether it is possible to replace the pRNA helical region with double-stranded siRNA, a variety of chimeric pRNAs were engineered. For this report, we found that replacement of pRNA's 5′/3′ helical region with siRNA or connection of the CD4-binding RNA aptamer, folate or other chemical components in this region did not interfere with the folding of the pRNA and siRNA or the function of the inserted moiety, nor did it impact the formation of trimers. It was found that three reagents engineered into the trimer were co-delivered simultaneously to specific cancer cells, guided by one of the chimeric pRNA building blocks that harbored the CD4-binding RNA aptamer or folate that bound the cell surface receptor.

Being able to use nanoparticles with sizes of 30-40 nm avoids the problem of the short half-life of small molecules in vivo due to short retention time. In addition, the problem of having molecules larger than 100 nm that are poorly delivered to cells is solved. It is well-accepted in the scientific community that RNA has a very low or undetectable level of immunogenicity except when complexed with protein. (Goldsby et al. In Immunology, 5th ed.; W. H. Freeman and Company: New York, 2002; pp 57-61; Madaio et al., J. Immunol. 1984; 132:872-876). Our system does not contain protein or peptides, and thus the use of such protein-free nanoparticles to avoid immune response would allow for long-term administration in the treatment of chronic diseases.

### Materials and Methods

Engineering of RNA building blocks and bottom-up assembly of pRNA trimer. RNAs were prepared as previously described (Shu et al., J Nanosci and Nanotech (JNN) 2003; 3:295-302). DNA oligos were synthesized with the desired sequences and used to produce double stranded DNA by PCR. The DNA products containing the T₇ promoter were cloned into plasmids or used as a substrate for direct in vitro transcription. All pRNA chimera produced by T7 RNA Polymerase were treated by Calf Intestinal Alkaline Phospatase to remove the 5′-phosphate. (Kim et al., Nat. Biotechnol. 2004; 22:321-325).

To engineer pRNA/siRNA(GFP), pRNA/siRNA(luciferase), and pRNA/siRNA(survivin), the helical region at the 5′/3′ paired ends of pRNA was replaced with double-stranded siRNA that connects to nucleotides #29 and 91.

To engineer chimeric pRNA harboring CD4-binding aptamer, the sequence of the RNA aptamer for CD4 binding (Kraus et al., J Immunol. 1998; 160(11):5209-5212) was connected to the 5′ and 3′ ends of pRNA. The pRNA was reorganized into a circularly permuted form, with the nascent 5′/3′-end relocated at pRNA nucleotides 71/75 located in a tightly-folded area. (Hoeprich et al., J Biol. Chem. 2002, 277(23);20794-20803). The chimeric pRNA with this CD4-binding aptamer retained the appropriate right and left loops, for example, loop A and b′ for pRNA A-b′, for the fabrication of trimer.

Physical characterization of the fabricated RNA nanoparticles. The structure and purity of the fabricated RNA nanoparticles were verified by: 1) 8% native polyacrylamide gel with 10 mM magnesium but without urea; 2) sedimentation by 5-20% sucrose gradient with 10 mM magnesium; and 3) AFM imaging. (Trottier et al., RNA 2000; 6:1257-1266; Mat-Arip et al., J Biol Chem 2001; 276:32575-32584; Chen et al., J Biol Chem 2000; 275(23):17510-17516).

Functional assay for engineered chimeric pRNA building blocks to cells. For proB FL5.12A cells, 10⁷ cells were resuspended in 500 μl RPMI with 10% FBS. For D1 cells, the cells were re-suspended in the hypo-osmolar buffer. Electroporation was performed with 4 mm cuvettes using an Electro Square Porator ECM 830. FL5.12A cells were electroporated at 200V, 3 pulses and D1 cells at 180V, 1 pulse. After a short incubation on ice, cells were resuspended in 10 ml complete media with cytokine and incubated for 2 hours at 37° C. and 5% CO₂. Cell viability was measured microscopically by Trypan Blue exclusion assay before transfection with Mirus reagent. 10⁶ cells were incubated with the RNA constructs (100 nM) overnight, washed twice with Hanks' balanced salt solution, re-suspended in the media with or without cytokine and incubated at 37° C.+5% CO₂ for an additional 24 to 48 hours.

For Drosophila S2 cells, GFP-expressing plasmid pMT-GFP and various siRNAs were co-transfected into cells in a 24-well plate using Cellfectin (Invitrogen) 24 hours after seeding. The expression of GFP was induced by overnight incubation with CuSO₄ at 0.5 mM 24 hours after transfection. Inhibition of GFP expression was observed by fluorescence microscopy.

Functional assays for delivery of the fabricated RNA nanoparticle to cells. For the IL-7-dependent D1 cell line, which was established from CD4− CD8− mouse thymocytes isolated from a p53−/− mouse, (Kim et al., J Immunol Methods 2003; 274:177-184) the cells were grown in complete medium containing RPMI1640 with 10% FBS (fetal bovine serum) and with penicillin and streptomycin at 50 U.I per ml, 0.1% beta-mercaptoethanol and 50 ng/ml IL-7. FL5.12A cells were grown in complete medium supplemented with 2 ng/ml IL-3. For overexpression of CD4 in the D1 cells, the L3T4 (mouse CD4) insert was subcloned into pcDNA 6/V5-HisB (Invitrogen). D1 cells were transfected with DNA by electroporation. Stable cell lines were selected by antibiotic resistance. D1-CD4^{hi} cells, expressing high levels of CD4, were further isolated by fluorescence-activated cell sorting. The D1-CD4^{hi} cell line was maintained in complete medium supplemented with 50 ng/ml IL-7 and Blasticidin HCl (2.5 mg/ml).

For assaying the delivery of folate-containing trimers, a human nasopharyngeal epidermal carcinoma KB cell line was maintained in folate-free RPMI1640 medium (Gibco BRL) supplemented with 10% FBS (fetal bovine serum) and penicillin and streptomycin in a 5% CO₂ incubator. The cells were grown into monolayers, and the serum provided the normal complement of endogenous folate for cell growth. Cell titer was determined by a hemocytometer after Trypan Blue straining.

The trimeric RNA complex was prepared by mixing pRNA(A-b′)/folate, pRNA(B-e′)/siRNA(GFP) and pRNA(E-a′)/siRNA(Firefly) in the same molar concentration. The trimer complex was then purified from gel and added into KB cells to allow the binding and entry of RNA. After washing with RPMI medium, the cells were collected and subjected to Dual reporter assay (Promega).

To assay for the delivery of chimeric pRNA complex to D1 cells, CD4^{hi}, CD4^{lo}, and CD4^{neg} cells were seeded in a 96-well flat bottom plate. 5×10⁴ cells per sample were washed once in PBS with 10 mM Mg²⁺. Cells were incubated for 30 min at 37° C. in 20 μl of PBS with 10 mM Mg²⁺ and 400 ng (100 nM) of RNA complex. After incubation, complete medium, with or without cytokines, was added to a final volume of 100 μl and cells were incubated at 37° C.+5% CO₂ for 24 or 48 hours. Cell viability was measured microscopically by Trypan Blue exclusion assay.

Chimeric pRNA/siRNA processing. The processing of RNA by cell lysate was carried out following the procedure used by Bernstein et al. (Bernstein et al., Nature 2001; 409 (6818):363-366).

For the processing of RNA by Dicer, the purified recombinant enzyme was purchased from Gene Therapy Systems.

Confocal microscopy. Coverslips coated with poly-L-lysine (200 μg/ml) were incubated overnight with cells grown in complete media supplemented with cytokines. CD4-negative pro-B cell line F15.12A (CD4^{neg}), pro-T cell line D1 (CD4^{neg}) and a D1-CD4 over-expressing cell line (CD4^{hi}) were incubated with the chimeric pRNA trimer. The trimer complex was purified from gel using the mixture containing the same molar RNA of pRNA(A-b′)/Aptamer(CD4), pRNA(B-e′)/FITC and pRNA(E-a′)/Rhodamine in the presence of 10 mM Mg²⁺. Cover slips with cells were fixed with 4% paraformaldehyde, washed in PBS with 10 mM Mg²⁺, and mounted in Gel/MountT (Biomeda, Calif.). The images were captured by Zeiss confocal microscope LSM 510 NLO.

In vivo animal trials. Five week old male athymic nude mice (Harlan Sprague Dawley) were housed in a pathogen-free environment. Animals were randomly assigned to experimental groups (n=8). Cells were maintained in antibiotic-free medium for one week before injection. On the day of injection, cells were rinsed with PBS twice and incubated with the dimeric pRNA complex at 37° C. for 3 h before being collected by trypsin digestion. 2.5×10⁵ cells in 0.1 ml of media were used for the injection of each mouse. Cells were inoculated subcutaneously at the right axilla of the forelimb. Once xenografts were visible, their size was determined two times per week by externally measuring tumors in two dimensions. Volume was calculated by the following equation: V=(L×W²)×0.5, where L is the length and W is the width of the xenograft.

### Results

Nomenclature of RNA building blocks: To simplify the description of bottom-up assembly using engineered RNA building blocks, uppercase letters will be used to represent the right hand loop of pRNA and lowercase letters to represent the left hand loop (FIG. 23). The same letters in upper and lower cases indicate complementary sequences for loop/loop interaction, while different letters indicate non-complementary loops. For example, pRNA A-b′ represents pRNA where right loop A (^{5′}G₄₅G₄₆A₄₇C₄₈) is complementary to left loop a′ (^{3′}C₈₅C₈₄U₈₃G₈₂) of pRNA E-a′(see Chen et al., RNA 1999; 5:805-818). The designation “pRNA/aptamer(CD4)” denotes a pRNA chimera that harbors an aptamer that binds CD4, while “pRNA/siRNA(survivin)” represents a pRNA chimera that harbors an siRNA targeting the anti-apoptosis factor survivin.

1. Engineering of Chimeric RNA as Building Blocks for the Fabrication of Trimers as Vehicles for the Delivery of Therapeutic Molecules.

First, the possibility of engineering chimeric pRNA building blocks harboring receptor-binding aptamer was tested. For specific delivery of therapeutic vehicles to cells, it is necessary to incorporate a chemical moiety that can recognize cell surface markers. In comparison to antibodies and phage-displaying peptides, RNA aptamer is an attractive alternative since it offers the advantage of avoiding the induction of immune responses. (Goldsby et al. Antigens. In Immunology, 5th ed.; W. H. Freeman and Company: New York, 2002; pp 57-61; Madaio et al., J. Immunol. 1984; 132:872-876). A powerful technique to obtain RNA aptamers that selectively bind to specific receptors with high affinity is based on in vitro screening of RNA molecules from a library that contains random RNA sequences (Ellington et al., Nature 1990; 346:818-822; Tuerk et al., Science 1990; 249:505-510). Using this SELEX approach, a number of aptamers have been obtained that specifically recognize a particular cell surface receptor such as CD4 (Kraus et al., J Immunol 1998; 160 (11):5209-5212). One CD4-binding RNA aptamer was incorporated into the pRNA via connection to its original 5′/3′ end (FIG. 23). The pRNA vector was engineered and reorganized into a circularly permuted form, with the nascent 5′ and 3′ end relocated to residues 71 and 75, respectively, of the original pRNA sequence. The 71/75 end has been shown to be located in a tightly-folded area (Hoeprich et al., J Biol. Chem. 2002; 277(23):20794-20803) which buries and protects the ends from exonuclease degradation (Hoeprich et al., Gene Therapy 2003; 10(15):1258-1267). The chimeric pRNA building block with this aptamer also contained the appropriate right and left loops required for the engineering and fabrication of the trimeric complex.

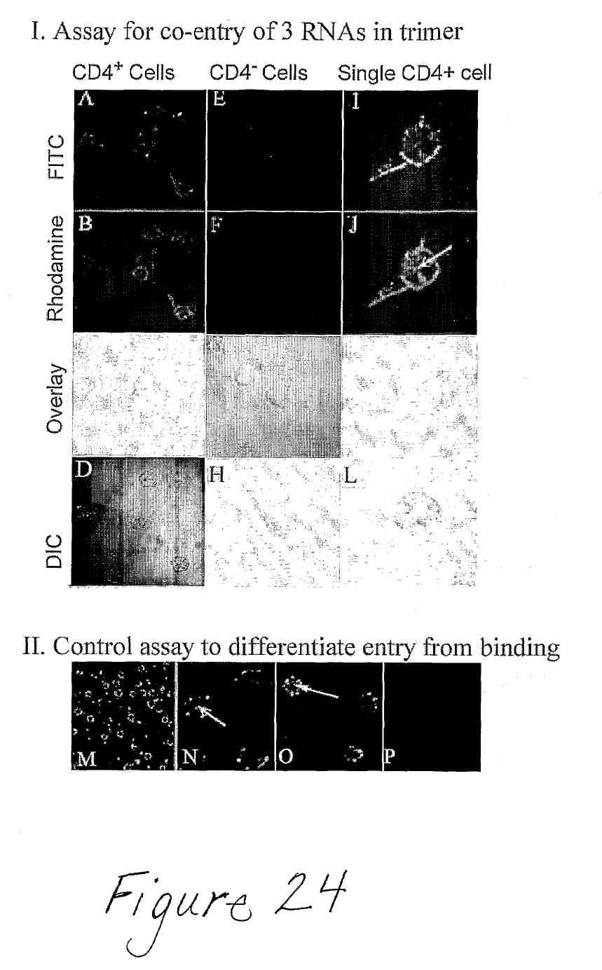

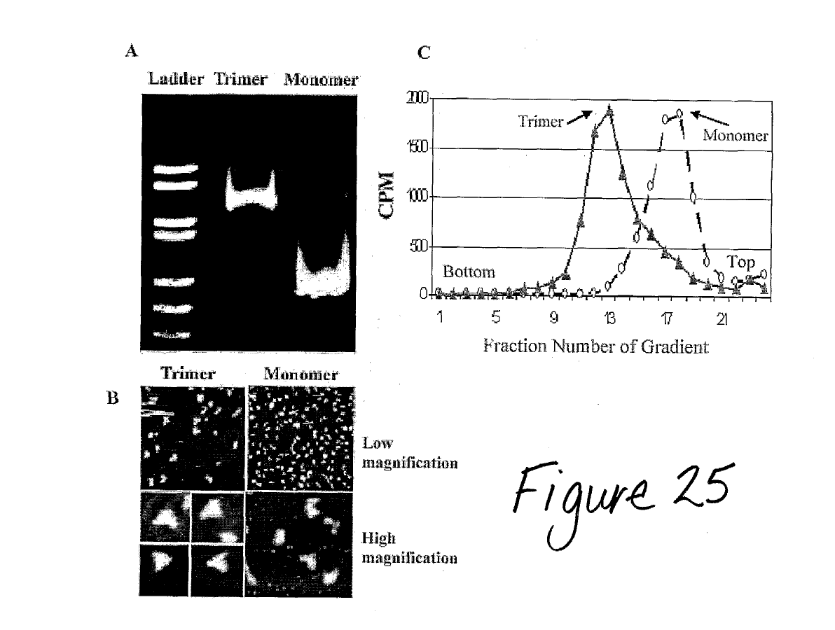

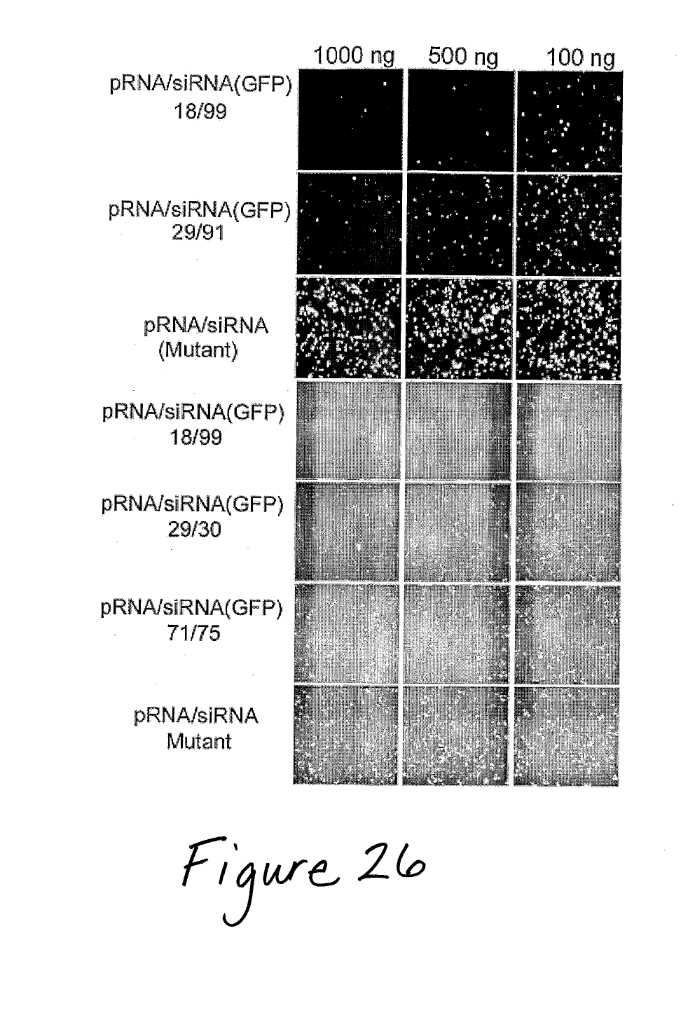

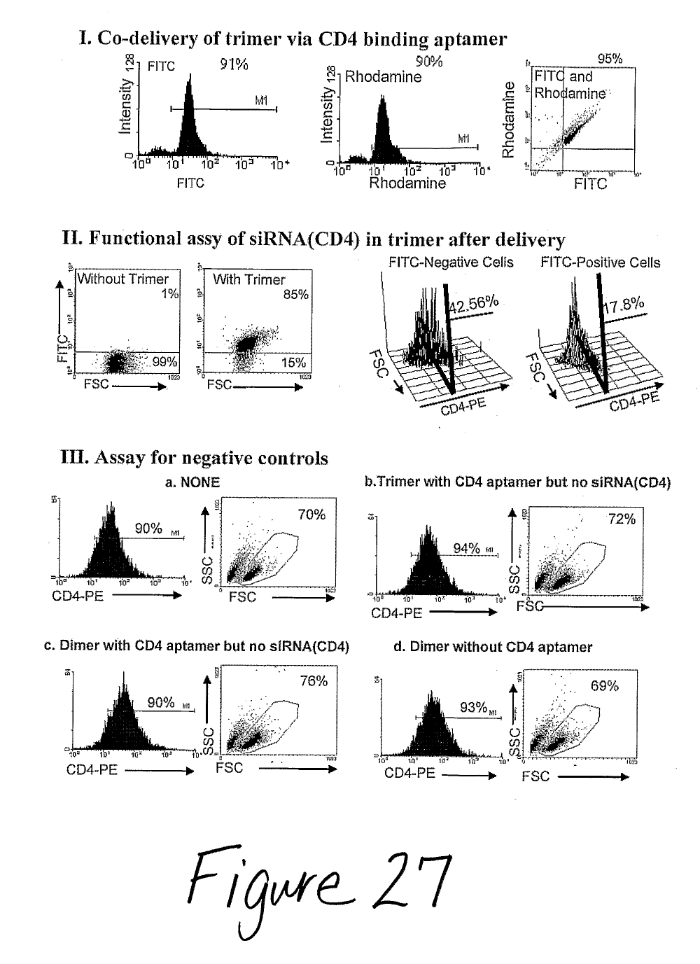

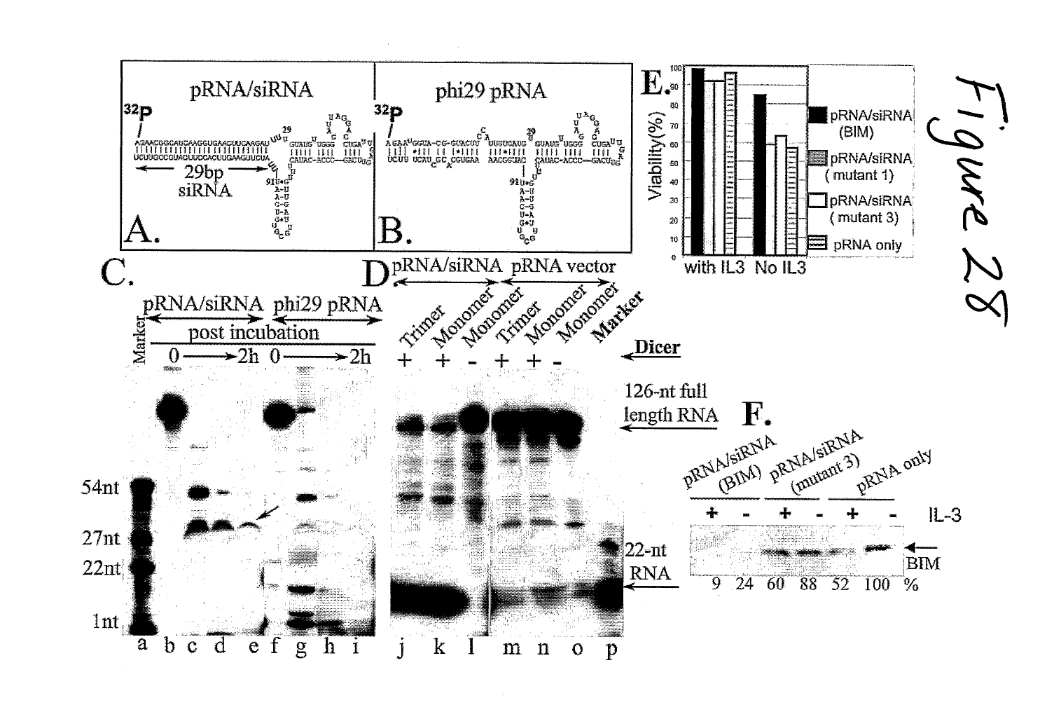

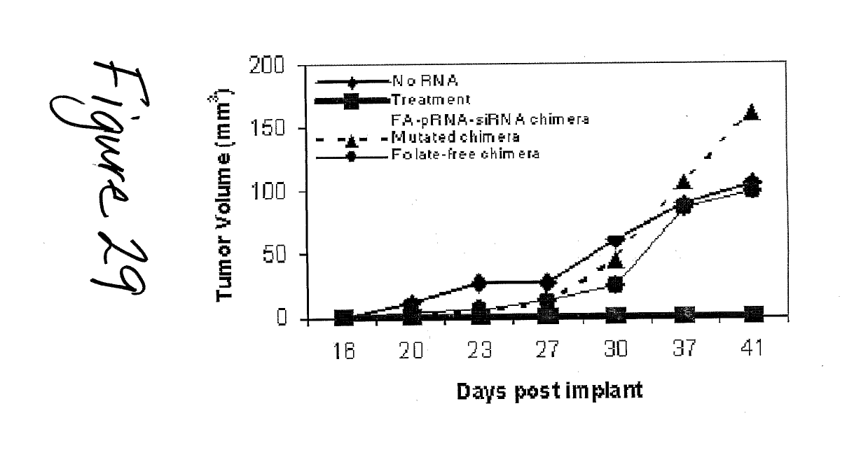

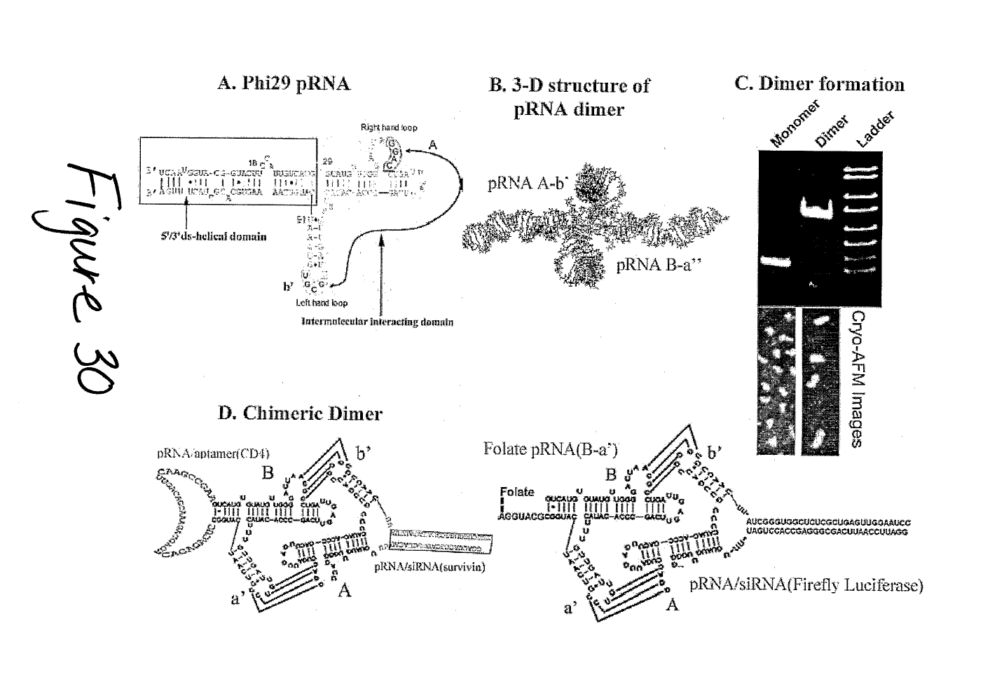

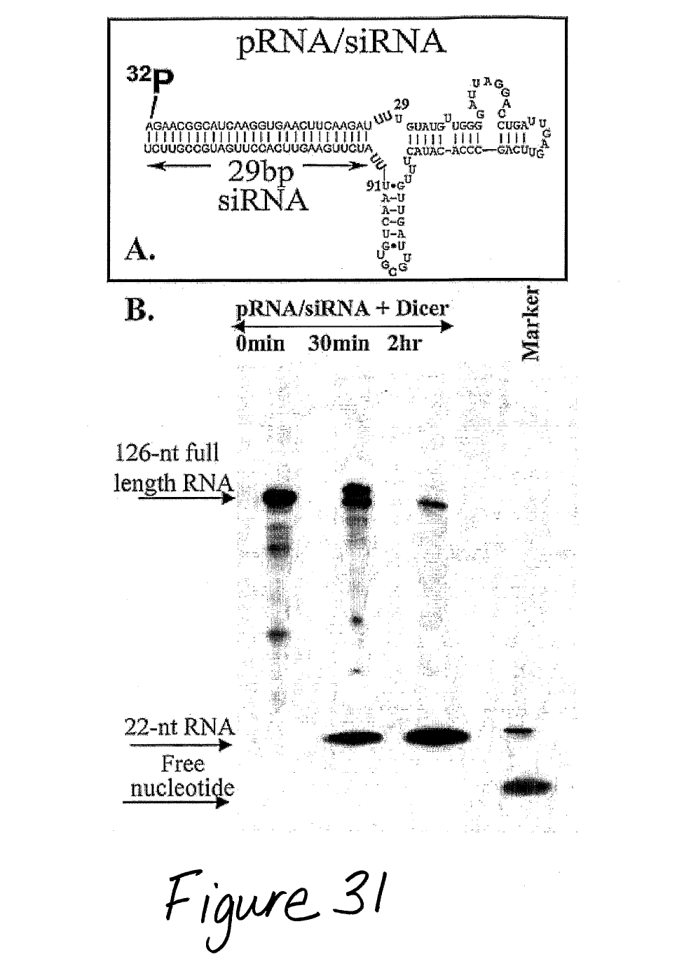

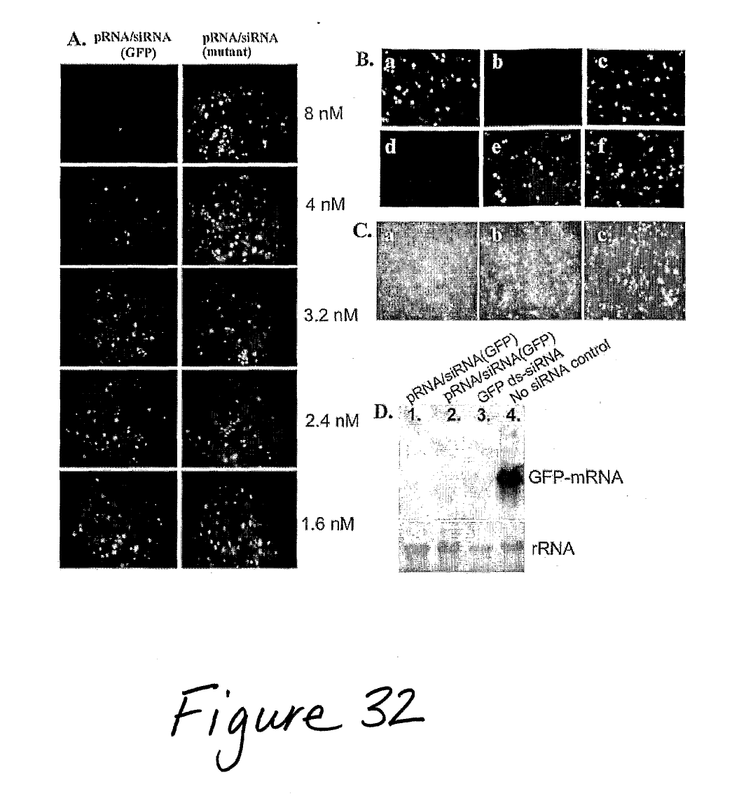

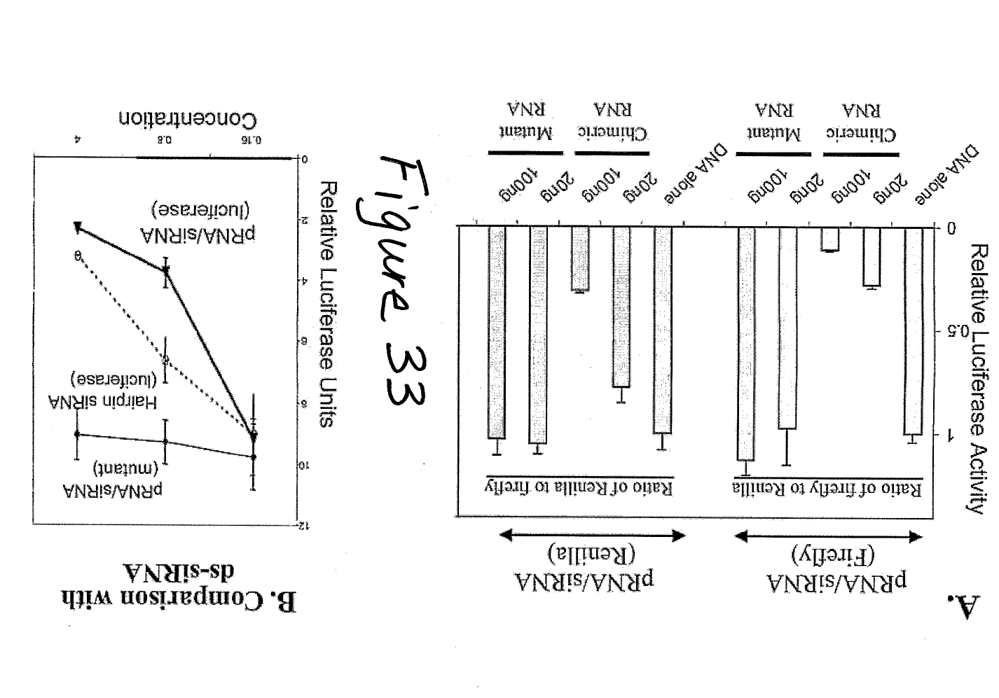

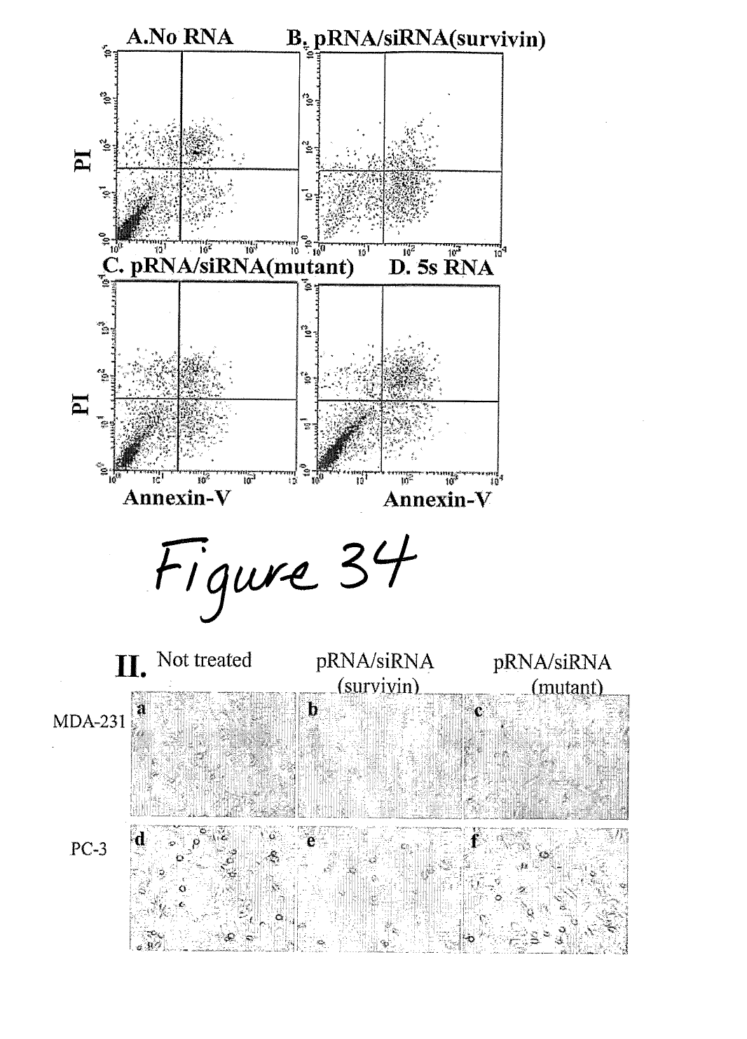

To test whether the pRNA/aptamer chimera was able to bind to specific cells, the pRNA/aptamer(CD4) was labeled with FITC and assayed for its ability to bind the CD4 receptor by fluorescent microscopy. Binding assay using a CD4-overexpressing engineered thymic T cell line, D1 (CD4^{hi}) (see below) and the CD4 negative parental line, D1 (CD4^{neg}) from which it was derived (Kim et al., J Immunol Methods 2003; 274:177-184) revealed that the chimeric pRNA-FITC/aptamer (CD4) was able to bind CD4^{hi} T cells efficiently. Binding and internalization was observed by confocal microscopy using the “Section” technique. A layer of T cells in the confocal microscope image displayed as a green circle, confirming binding of the FITC-label chimeric pRNA/aptamer(CD4) to the spherical T cells (FIG. 2-II). The binding specificity of the aptamers was investigated using a variety of controls, including CD4 receptor-negative D1 cells (FIGS. 24E, F, G, H, P), fluorescent pRNA dimer or pRNA trimer without CD4 aptamer (not shown), and fluorescent dye alone without RNA (not shown)—all of which resulted in minimal fluorescence detection. Both CD4^{hi} and CD4^{neg} internalized the transferrin—Texas Red positive control, suggesting that the cells were competent for the endocytosis of membrane-bound molecules (FIG. 24N).

Then, the engineering of chimeric pRNA building blocks harboring receptor-binding folate was also tested. Folate receptors are over-expressed in various types of tumors and have been used to deliver therapeutic reagents specifically into cancer cells (Zhao et al., Adv. Drug Deliv. Rev. 2004; 56:1193-1204; Lu et al., Adv. Drug Deliv. Rev. 2002; 54:675-693). Therefore, selective targeting of folate-conjugated pRNA nanoparticles to the folate receptor is a useful method for targeting cancer cells. A chimeric RNA harboring pRNA and folate (folic-pRNA) was engineered by covalently linking a folate to the 5′-end of the pRNA. Specific cell binding of folic-pRNA was demonstrated by flow cytometry using a folic-pRNA labeled with FITC. When folate receptor-positive human nasopharyngeal epidermal carcinoma cells were incubated with this dually labeled pRNA, nearly all of the cells were FITC-positive. Adding free folate to the incubation buffer decreased the FITC positive cells to less than 1%, suggesting that the binding of pRNA to cells is specific and folate-dependent. As a control, FITC-labeled pRNA without folate conjugation did not exhibit binding.

2. Determination of the Sequence Requirement of the Hand-in-Hand Loops in Chimeric Trimer Formation.

Bases 45-48 and 82-85 of wild type pRNA in the left- and right-hand loops, respectively, were found to engage in pRNA/pRNA interactions (Guo et al., Mol. Cell. 1998; 2:149-155; Trottier et al., RNA 2000; 6:1257-1266). Without considering tertiary interaction, in some cases only two GIC pairs between the interacting loops could allow the formation of pRNA multimers. When all four nucleotides were paired, at least one G/C pair was required. The maximum number of base pairings between the two loops to allow optimal multimer formation was five. The minimum number of nucleotides needed for pRNA/pRNA interaction in the right and left loop was five and three, respectively. Our results suggest that a 75-nucleotide RNA segment, nucleotide 23-97, is a self-folded independent domain involved in RNA/RNA interaction in pRNA trimer formation, while nucleotide 1-22 and 98-120 were dispensable for RNA/RNA interaction.

The mechanism of pRNA trimer formation by interlocking loop/loop interaction was utilized for the fabrication of the trimer of chimeric pRNA harboring receptor-binding RNA aptamer and/or therapeutic siRNA (FIG. 23). Individual chimeric pRNA building block was engineered to carry one daughter RNA molecule such as siRNA or receptor-binding aptamer. Each building block was intentionally designed to have specific right or left loops, such as A-b′ (right-left), to interact with other building block. The possibility of appropriate folding of pRNA and their competency in forming trimers were tested. Mixing of individual chimeric pRNAs with counterpart partners with appropriate interlocking loops resulted in the efficient formation of the desired trimer, as documented by gel electrophoresis, AFM imaging, and sucrose gradient sedimentation (FIG. 25). The monomer building block migrated more rapidly in native gels, whereas the trimer complex composed of three chimeric pRNA (A-b′), (B-e′), and (E-a′) (FIG. 23-III) migrated more slowly in native gels (FIG. 25A). This suggests that RNA trimers were generated from the monomeric building block despite the replacement of the 5′/3′ helix with ds-siRNA or the connection of the 5′/3′ end to a CD4-binding aptamer. The correct folding of pRNA chimera was also confirmed by phi29 in vitro assembly inhibition assay, (Guo et al., Science 1987; 236:690-694; Guo et al., Mol. Cell. 1998; 2:149-155) based on the finding that correct folding of pRNA chimeras will ensure competitive binding to phi29 procapsid for the pRNA binding site and will block phi29 DNA packaging during phi29 replication.

3. Determination of the Length Requirement of the pRNA Vector for the Construction of pRNA Chimera Harboring siRNA

As noted earlier, the intermolecular interacting domain and the double-stranded helical domain of pRNA fold independently (FIG. 23). Altering the primary sequence of any nucleotide of the helical domain does not impact pRNA structure and folding so long as the complementarity is preserved. Additional functional RNAs, such as a hammerhead ribozyme, have been conjugated to the double-stranded domain, resulting in enhanced cleavage efficiency of ribozyme. (Hoeprich et al., Gene Therapy 2003; 10(15):1258-1267)

Extensive studies reveal that siRNA is a double-stranded RNA helix (Li et al., Science 2002; 296:1319-1321; Brummelkamp et al., Science 2002; 296:550-553; Jacque et al., Nature 2002; 418:435-438; Carmichael et al., Nature 2002; 418:379-380; Elbashir et al., Nature 2001; 411L494-498). To test whether it is possible to replace the pRNA helical region with double-stranded siRNA and to determine which construct has the optimal function in gene silencing and trimer formation, several chimeric pRNA/siRNAs were constructed, and their effectiveness in gene silencing was tested. A 29-bp siRNA was connected to nucleotides 29/91 or 21/99 of pRNA, resulting in pRNA/siRNA(GFP)29/91 and pRNA/siRNA(GFP)21/99, respectively. Two additional uridines were inserted into the three-way junction to increase the flexibility at this region for RNase processing. As revealed by fluorescent microscopy, both chimeric siRNAs showed significant inhibition against GFP expression after introduction into cells by transient transfection (FIG. 26). The inhibition was highly specific since a mutant chimeric pRNA/siRNA with mutations at the siRNA sequence did not exhibit any inhibitory effects.

Circularly permuted pRNA chimeras were also constructed to carry siRNA. For these constructs, their 5′/3′ ends were relocated within a tightly folded region and therefore not easily accessed by exonuclease, thus increasing the stability of the entire RNA (Zhang et al., Virology 1995; 207:442-451). Two circularly permuted pRNA/siRNA chimera, with new 5′/3′ termini located at 71/75 or 29/30 positions of original pRNA, were constructed and referred to as pRNA/siRNA(GFP)71/75 and pRNA/siRNA(GFP)29/30. pRNA/siRNA(GFP)29/30 silenced GFP expression with lower efficiency than pRNA/siRNA(GFP)21/99, while pRNA/siRNA(GFP)71/75 had virtually no effect on silencing GFP expression (FIG. 26).

In addition, chimeric pRNA harboring siRNA for luciferase were also constructed (FIG. 23). Dual reporter assay demonstrated that pRNA/siRNA(Firefly) strongly and specifically inhibited the expression of firefly luciferase proteins without affecting renilla luciferase expression. Specific knockdown was also observed for pRNA/siRNA(Renilla).

4. Demonstrating the Co-Delivery of Three pRNA Chimeric Building Blocks into One Cell

a. Demonstrating Co-Delivery by Confocal Microscopy and Flow cytometry. CD4 is a receptor displayed on the surface of certain subsets of T lymphocytes. In T helper cells, CD4 is normally not involved in endocytosis except when overexpressed. (Pelchen-Matthews et al., J. Exp. Med. 1991; 173:575-587). A murine thymic T lymphocyte cell line D1 (Kim et al., J Immunol Methods 2003; 274:177-184) which depends on IL-7 for growth, was used as a model system for testing the effects of specific gene delivery via CD4. Since D1 cells are immature thymic cells that minimally express CD4, we overexpressed murine CD4 in D1 cells by electroporating them in hypotonic buffer in the presence of a mammalian expression vector containing L3T4 (mouse CD4). Antibiotic selection was used to screen out integrations, and the cells that expressed high levels of CD4 (CD4^{hi}) were further selected by fluorescence-activated cell sorting (FACS). As a result, more than 99% of the CD4^{hi} cells expressed the CD4 receptor.

To test whether this RNA nanoparticle could serve as a vehicle to concurrently and specifically deliver multiple therapeutic molecules, a trimeric complex composed of pRNA(A-b′)/aptamer(CD4), pRNA(B-e′)/FITC and pRNA(E-a′)/Rhodamine (FIG. 23, III-C) was fabricated and assayed by the section technique using confocal microscopy (FIG. 24). Binding of the trimer and the co-entry of three chimeric building blocks into the cell via CD4 binding was demonstrated by detection of fluorescence within CD₄^{hi} cells (FIG. 24, A-D & I-L)). Such binding and entry was specific since no fluorescence was observed on CD4^{neg} cells (FIG. 24, E-H). When an FITC filter was used, the green fluorescent label was visible in the CD4^{hi} T lymphocytes (FIGS. 24 A & I). When a rhodamine filter was used, the red label was visible on the image that overlaps with the FITC label in the CD4^{hi} T lymphocytes (FIGS. 24, B & J). The similarity and overlap of the FITC image with the rhodamine image, (FIGS. 24, C & K) and the co-appearance of both green and red, representing FITC and rhodamine, respectively, on the same location of the cell confirmed the co-delivery of three chimeric pRNA building blocks into specific cells with the aid of CD4-binding RNA aptamer. These results indicate that all of the pRNA building blocks were co-delivered as a trimer, and that the phi29 pRNA trimer can serve as an effective vehicle for the delivery of multiple therapeutic components.

The co-delivery of three components in the fabricated trimer was further confirmed by analyzing a large population of cells (30,000 cells) using flow cytometry (FIG. 27-I). Dual incorporation of both FITC and Rhodamine in 90-95% of the CD4^{hi} T cells indicates successful uptake of the pRNA trimer by almost all of the CD4-expressing cells in the population.

b. Functional assay for the co-delivered trimer harboring pRNA/siRNA(CD4), pRNA/aptamer(CD4) and pRNA/FITC. Having demonstrated co-delivery of three components of the pRNA trimer (FIG. 24), it is necessary to test if uptake of the pRNA trimer resulted in a biological effect mediated by the delivered components.

CD4^{hi} and CD4^{neg} D1 T cells were incubated, but not transfected, with pRNA trimer containing the building blocks of pRNA/siRNA(CD4), pRNA/aptamer(CD4) and pRNA/FITC (FIG. 23). Inhibition of CD4 expression by siRNA(CD4) incorporated in the trimer was demonstrated by measuring surface expression of CD4 with a PE-labeled CD4 antibody. CD4^{neg} D1 cells did not take up detectable amounts of the pRNA trimer when assessed for FITC uptake. This was confirmed by flow cytometry (data not shown). In contrast, 85% of CD4^{hi} D1 cells treated with the pRNA trimer were FITC-positive (FIG. 27-II), demonstrating that the CD4 aptamer was selectively targeting CD4-overexpressing cells and delivering the pRNA trimer complex.

Next, to determine whether the siRNA(CD4) delivered by the pRNA trimer complex resulted in the down-regulation of CD4 surface expression, CD4^{hi} D1 cells were incubated with the trimer for 24 hours, and then stained with anti-CD4 antibody conjugated with PE and subjected to flow cytometry analysis. FITC-positive and FITC-negative cells were gated from the total cell population (FIG. 27-II). The CD4 level in FITC-negative cells was determined to be 42.56%, but the level was reduced to 17.8% in FITC-positive CD4^{hi} D1 cells. Therefore, only in FITC-positive cells (85% of the CD₄^{hi} D1 cells) but not FITC-negative cells (15% of the CD4^{hi} D1 cells) were the level of CD4 expression decreased to such an extent. These findings suggest that in CD4-expressing cells the pRNA trimer is internalized, as indicated by the uptake of pRNA/FITC, and is functional in silencing the CD4 gene, as indicated by the co-delivery of pRNA/siRNA(CD4) complex resulting in decreased expression of the CD4 protein in FITC-positive cells.

Extensive study (FIG. 27-III) with controls indicated that the reduction of CD4 level in PE staining seen in FIG. 27-II is specifically due to gene silencing by pRNA/siRNA(CD4) in the pRNA trimer and is not caused by CD4-binding-triggered internalization, since CD4 aptamer containing pRNA complexes did not cause CD4 reduction unless the CD4 siRNA was incorporated.

c. Functional assay for co-delivery of siRNAs targeting anti- or pro-apoptosis factors guided by CD4-binding RNA aptamer. To evaluate the effectiveness of the preferential delivery system, an RNA nanoparticle harboring siRNA targeting pro- or anti-apoptotic factors was engineered. Survivin is an anti-apoptotic factor that is not expressed in most normal adult human tissues but is expressed in most human cancers (Grossman, Proc Natl Acad Sci 2001; 98:635-640; Ambrosini et al., J Biol Chem 1998; 273 (18):11177-11182; Choi et al., Cancer Gene Ther. 2003; 10 (2):87-95). pRNA/siRNA(survivin) was tested to evaluate the usefulness of inducing apoptosis as a therapeutic means for killing cancer cells. For comparison, chimeric pRNA/siRNAs targeting pro-apoptosis factors, with BAD and BIM as examples, were designed and tested. By preventing apoptosis instead of inducing it, the pro-apoptosis assay could more clearly determine if the delivery of the chimeric pRNA complex produced a non-specific toxic response. To this end, two cytokine-dependent cell lines were employed—an IL-3 dependent pro-B cell line (FL5.12A) and the CD4^{hi} or CD4^{neg} IL-7-dependent D1 cell line. Withdrawal of IL-3 or IL-7 could promote the apoptosis of D1 or the FL5.12A cell line, respectively, and induce the expression of BAD and BIM in FL5.12A cells. (Letai et al., Cancer Cell 2002; 2:183-192; Zong et al., Genes Dev. 2001; 15:1481-1486; Liu et al., Cancer Res. 2002; 62:2976-2981; Simoes-Wust et al., Breast Cancer Res Treat. 2002; 76(2):157-166; Luzi et al., Cancer Gene Ther. 2003; 10 (3):201-208; Kumar et al., Science 2002; 297 (5585):1290-1291; Gibson et al., Clin. Cancer Res 2006: 213-222; Khaled et al., Nat. Rev. Immunol. 2002; 2: 817-830)

Extensive testing of the RNA nanoparticles, including monomer (by transfection), dimer (by transfection or incubation) and trimer (by incubation) confirmed the specific binding and entry of pRNA complexes guided by the CD4-binding RNA aptamer. An annexin V-propidium iodide (PI) double-staining method followed by flow cytometry was carried out 12, 24, 48 and 120 hrs after transfection confirmed the induction of apoptosis by pRNA/siRNA(survivin) in MCF-7 cells (data not shown). It was found that pRNA/siRNA(survivin) and pRNA/siRNA(BIM) silenced their target genes specifically and efficiently. In addition, the introduction of pRNA/siRNA(survivin) caused cancer cells and cytokine-dependent cells to die, while the introduction of pRNA/siRNA(BIM) protected IL-3-dependent cells from death in the absence of IL-3 (FIG. 28).

d. Co-delivery of pRNA/siRNA(Renilla) or pRNA/siRNA(Firefly) in trimers guided by folate-pRNA. The co-delivery of three components to specific cells conveyed by the RNA nanoparticle was further demonstrated using trimers harboring pRNA/siRNA(Firefly or Renilla). Using dual report assay, one of the luciferases, for example renilla luciferase, will actively serve as an internal control for the other luciferase, e.g. firefly luciferase. The mutual internal control could prove the specific activity of siRNA by eliminating the possible nonspecific effect. By incubation of the human nasopharyngeal carcinoma cells expressing folate receptors on their surface with trimer, reduction of the specific luciferase activity was demonstrated by the dual assay system, confirming the specific delivery of the trimer into the cells guided by folate-pRNA.

5. Mechanism of Action, Processing of Chimeric pRNA/siRNA Complex into Individual Double-Stranded siRNA by Cellular Components or Dicer.

The chimeric pRNA/siRNA complex functions within the cell in a role similar to specific siRNA in gene silencing. This raises an important question: are the chimeric complexes processed into individual siRNA? To address this question, the chimeric pRNA/siRNA monomer or the trimeric chimera was incubated with cell lysates (FIG. 28A-D) and analyzed by denatured gel. The monomer in this study harbored a 29-nucleotide double-stranded siRNA connected to the three-way junction from nucleotides 29 to 91 (FIG. 28A). Two additional uridines were added to the UUU bifurcation bulge to help enhance processing efficiency by increasing the ΔG for the folding of the loop. Incubation with cell lysates resulted in a band with a size equal to 29-nucleotide double-stranded RNA (FIG. 28A, C lane c-e), suggesting that the siRNA was released after cleavage in the cell lysates by RNase as expected, since the single-stranded bifurcation bulge is much more susceptible than the double-stranded RNA to RNase digestion.

Incubation of the purified trimeric chimera with purified Dicer, a cell component responsible for the processing of siRNA, resulted in a band with a size equal to 21-nucleotide double-stranded siRNA, suggesting that the gene silencing effect induced by the trimer is indeed caused by siRNAs after the processing of the trimer by the Dicer in the cell.

6. Animal Trials to Demonstrate Specific Delivery of the Therapeutic pRNA/siRNA Complex to Cancer Cells Conveyed by the RNA Nanoparticle.

Animal trials were conducted to test the specificity in delivery of the pRNA complex containing a pRNA building block labeled with folate and a pRNA building block carrying survivin siRNA. The potential of this RNA complex to suppress tumor formation was tested in athymic nude mice. Human nasopharyngeal epidermal carcinoma cells were incubated with a chimeric RNA complex with or without folate before being introduced into the nude mice by axilla injection. The mice receiving only cancer cells developed tumors within 3 weeks, while the group of mice that received cancer cells pre-treated with the pRNA complex containing both folate-pRNA and pRNA/siRNA(survivin) did not develop tumors (FIG. 29). The inhibition of tumor formation is specific since the control RNA complex without folate or the RNA complex containing mutations in survivin siRNA did not affect tumor development in other mice groups.

### Discussion

We have reported that phi29 pRNA, via the interaction of programmed helical regions and loops, can be engineered and fabricated at will to form a variety of structures and shapes, including twins, tetramers, rods, triangles and arrays with sizes ranging from nm to microns (Shu et al., J Nanosci and Nanotech (JNN) 2003; 3:295-302; Shu et al., Nano Letters 2004; 4:1717-1724). Such fabricated RNA nanoparticles could hold diverse RNA building blocks with controllable stoichiometries ranging from one, two, three or six copies up to thousands of copies. The beauty of this system is that the size, shape and stoichiometry of the building blocks and the final product are capable of being manipulated and controlled. The features of multiplicity and assortment make such RNA nanoparticles capable of carrying polyvalent therapeutic molecules to enhance therapeutic efficacy. Using one complex to carry out the actions of several molecules will solve the problem of developing multiple factors for a specific therapeutic strategy. We have reported here the co-delivery of three components using the mechanism of pRNA trimer formation. Co-delivery of six components in a hexameric complex is also possible, since pRNA forms hexamers as well (Guo et al., Mol. Cell. 1998; 2:149-155; Zhang et al., Mol. Cell. 1998; 2:141-147; Hoeprich et al., J Biol. Chem. 2002; 277(23):20794-20803). One building block of the deliverable RNA complex can be modified to carry an RNA aptamer that binds a specific cell-surface receptor, thereby inducing receptor-mediated endocytosis. The second building block of the hexamer will carry heavy metal, quantum dots, fluorescent beads, or radioisotopes for cancer detection. The third building block of the hexamer will be altered to carry components that will be used to enhance endosome disruption so that the therapeutic molecules are released. The fourth and fifth building blocks of the RNA complex will carry therapeutic siRNA, ribozyme RNA, antisense RNA or other drugs to be delivered. A sixth building block of the hexamer will be designed to allow for the detection of apoptosis. Nucleotide derivatives such as 2′-F-2′ deoxy CTP, 2-F-2′ deoxy UTP or spiegelmer would be incorporated into the RNA to produce stable in vitro RNA transcripts that are resistant to RNase digestion.

In addition to cancer therapy, this polyvalent RNA complex can also be used for treating chronic viral infections such as those caused by HIV and HBV (Hepatitis B virus) through targeting at the specific virus-glycoproteins incorporated on the cell surface of infected cells. The development of specific recognition methods that do not produce serious side effects by damaging healthy cells would dramatically improve the treatment of HIV, HBV and other related chronic and latent diseases. RNA is uniquely suitable for chronic diseases since it has low or undetectable antigenicity (Goldsby et al. Antigens. In Immunology, 5th ed.; W. H. Freeman and Company: New York, 2002; 57-61; Madaio et al., J. Immunol. 1984; 132:872-876). The use of such a 30- or 40-nm RNA complex will provide a longer turnover time in the body than other small molecules would offer.

### Example 12

### Specific Delivery of Therapeutics to Cells Via the Dimerization Mechanism of Phi29 Motor pRNA

The application of small RNA in therapy has been hindered by the lack of an efficient and safe delivery system to target specific cells. The motor pRNA of bacteriophage phi29 was manipulated using RNA nanotechnology to make chimeric RNAs that form dimers via interlocking right and left hand loops. Fusing pRNA with receptor-binding RNA aptamer, folate, siRNA, ribozyme, or other chemical groups did not disturb dimer formation or interfere with the function of the inserted moieties. Incubation of the cells with the pRNA dimer, one subunit of which harbored the receptor-binding moiety and the other harboring the gene silencing molecule, resulted in their binding and entry into cancer cells, and subsequent silencing of anti-pro-apoptotic genes. The chimeric pRNA complex was found to be processed into functional double-stranded siRNA by Dicer. Animal trials confirmed the suppression of tumorigenicity of cancer cells by ex vivo delivery. Such protein-free 30 nm nanoparticles will allow for repeated long-term administration and avoid problems of short retention time of small molecules less than 30nm and undeliverability of particles larger than 100 nm.

We discovered a 117-nt bacteriophage phi29-encoded RNA (pRNA) that plays a novel and essential role in packaging DNA into procapsids (FIG. 30) (Guo et al. (1987), Science, 236, 690-694). Six copies of pRNA form a hexameric ring to drive the DNA-packaging motor (Trottier & Guo (1997), J Virol, 71, 487-494; Guo et al. (1998), Mol Cell, 2, 149-155; Zhang et al. (1998), Mol Cell, 2, 141-147). pRNA dimers are the building blocks of hexamers (Chen et al. (2000), J Biol Chem, 275(23), 17510-17516). Hand-in-hand interaction of the right and left interlocking loops can be manipulated to produce desired stable dimers, trimers or hexamers (Guo et al. (1998), Mol Cell, 2, 149-155; Zhang et al. (1998), Mol Cell, 2, 141-147; Chen et al. (1999), RNA, 5, 805-818; Shu et al. (2003), J Nanosci and Nanotech (JNN), 3, 295-302). The size of pRNA dimer is around 30 nm (Hoeprich & Guo (2002), J Biol Chem, 277(23), 20794-20803; Shu et al. (2004), Nano Letters, 4, 1717-1724). Our recent work indicates that RNA, and especially pRNA, can serve as a building block to build nanomaterials via bottom-up assembly (Shu et al. (2004), Nano Letters, 4, 1717-1724). The structural and molecular features of phi29 pRNA allow its easy manipulation, making it possible to redesign its parts as gene targeting and delivery vehicles. The pRNA molecule contains intermolecular interaction domains and a 5′/3′ helical domain (FIG. 30) (Zhang et al. (1995), RNA, 1, 1041-1050; Garver et al. (1997), RNA, 3, 1068-1079; Chen et al. (1999), RNA, 5, 805-818; Chen et al. (2000), J Biol Chem, 275(23), 17510-17516). Replacement or insertion of the 5′/3′ helical domain does not interfere with dimer formation (Chen et al. (1999),RNA, 5, 805-818).

The feasibility of these ideas was tested by the construction of chimeric pRNA dimers. One subunit of the dimer contained a receptor-binding RNA aptamer or folate for cell recognition, and the other harbored a moiety of siRNA, ribozyme or chemical groups. The dimers were delivered to specific cells to silence the genes for GFP, luciferase and pro/anti-apoptotic members of the BCL-2 family in a variety of cancer cells.

### Materials and Methods

In vitro synthesis and physical characterization of RNA RNAs were prepared as described (Zhaang et al. (1995), RNA, 1, 1041-1050) DNA oligos were synthesized with the desired sequences and used to produce double stranded DNA by PCR. The DNA products containing the T7 promoter were cloned into plasmids or used as a substrate for direct in vitro transcription. All pRNA chimera were treated by Calf Intestinal Alkaline Phosphatase (CIP) to remove the 5′-phosphate and eliminate PKR and interferon effect (Kim et al. (2004), Nat Biotechnol, 22, 321-325) or synthesized in the presence of SH-AMP, Biotin AMP, or CoA. Methods of electrophoresis and cryo-AFM have been described (Chen et al. (2000), J Biol Chem, 275(23), 17510-17516; Shu et al. (2003), J Nanosci and Nanotech (JNN), 3, 295-302). 10 mM of magnesium was included in all buffers to maintain the folding of pRNA and the formation of dimers (Chen et al. (2000), J Biol Chem, 275(23), 17510-17516; Mat-Arip et al. (2001), J Biol Chem, 276, 32575-32584).

Transfection Assay for Monomeric Chimeric pRNA Subunits

For Drosophila S2 cells, various siRNAs and GFP-coding plasmid pMT-GFP were co-transfected in a 24-well plate using Cellfectin (Invitrogen). The expression of GFP was induced by overnight incubation with CuSO₄ at 0.5 mM (Li et al. (2002), Science, 296, 1319-1321).

For luciferase assay of monomer pRNA/siRNA, various chimeric siRNA were co-transfected into mouse fibroblast PA317-PAR cells with both plasmid DNA pGL3 coding firefly luciferase and pRL-TK (Promega) coding Renilla luciferase. Luciferase activities were measured in dual reporter assay system (Promega) one day after transfection.

For survivin knockdown assay, MDA-231, PC-3, A-549, T47D and MCF-7 cells were transfected with various RNAs at 20 pmols per well in 24-well-plates using Lipofectamine2000 (Invitrogen). The following day, cells were observed under a phase contrast microscope and scored based on viability.

For proB FL5.12A cells, 10⁷ cells were resuspended in 500 μl RPMI1640 with 10% FBS. For D1 cells, the cells were re-suspended in the hypo-osmolar buffer. Electroporation was performed using an Electro Square Porator ECM 830. FL5.12A cells were electroporated at 200V, 3 pulses and D1 cells at 180V, 1 pulse. After a short incubation on ice, cells were resuspended in 10 ml complete media with cytokine and incubated for 2 hours. Cell viability was measured by trypan blue assay before transfection with Mirus reagent. 10⁶ cells were incubated with the RNA constracts(100 nM) overnight, washed twice with Hanks' balanced salt solution, and incubated for an additional 24 to 48 hours in the medium with or without cytokine.

Chimeric pRNA/siRNA Processing by Dicer

Chimeric siRNA was incubated with purified recombinant Dicer(Gene Therapy Systems, Inc.) for 2 hour at 37° C. RNA was labeled with [³²P] at 5′-end using T4 Polynucleotide Knase(NEB).

Functional Assays for Chimeric pRNA Dimer Harboring CD4 Aptamer and Survivin siRNA

CD4^{hi}, CD4^{lo} and CD4^{neg} cells were seeded in a 96-well plate. 5×10⁴ cells per sample were washed once. Cells were incubated with 100 nM of RNA dimer for 30 min. After rinse, cells were further incubated for 24 or 48 hours, with or without cytokines. Cell viability was measured by trypan blue assay.

### CD4 Receptor-Binding Assay by Confocal Microscopy

D1 cells were grown in RPMI1640 with 10% FBS, with penicillin/streptomycin 50 U.I per ml, 0.1% beta-mercaptoethanol and 50 ng/ml IL-7. FL5.12A cells were grown in complete medium supplemented with 2 ng/ml IL-3. For the expression of CD4 in D1 cells, the L3T4 (mouse CD4) insert was subcloned into pcDNA 6/V5-HisB (Invitrogen). Stable lines selected by antibiotic resistance. D1-CD4 cells, expressing high levels of CD4, were further isolated by FACS. The D1-CD4 cell line was maintained in complete medium supplemented with 50 ng/ml IL-7 and Blasticidin HCl (2.5 mg/ml). Coverslips coated with poly-L-lysine (200 μg/ml) were incubated overnight with cells. Prior to fixing, cover slips with cells were rinsed and treated for 30 minutes in a 65 nM solution of dimeric RNA complex. Cover slips with cells were fixed with 4% paraformaldehyde and mounted in Gel/MountT (Biomeda, Calif.). The images were captured by Zeiss confocal microscope LSM 510 NLO.

### Specific Gene Knockdown Mediated by Folate Recptor

Folate-dimer was prepared by mixing folate-pRNA(7-106) B-a′ and pRNA/siRNA(Firefly luciferase) A-b′ with 10 mM Mg²⁺. KB cells were seeded in a 6-well plate in folate-free medium. After being washed by PBS-supplied MgCl₂, the pre-mixed dimer RNA(1.75 uM) was then added to cells and incubated for 3 h at 37° C. RNase inhibitor SUPERRNaseIN (1 unit/ul) (Ambion) was added into the binding buffer. After incubation, free RNA was washed off and pGL3 and pRL-TK plasmids were introduced into cells using Lipofectamine 2000 (Promega). Luciferase activities were measured the next day.

### Double-Labeling and Flow Cytometry

MCF-7 cells were transfected with RNA samples at a 100 nM concentration. Cells were stained by annexin V and PI followed by flow cytometry assay. The upper left, upper right, lower left and lower right area represents cells destroyed, necrotic cells, viable cells and apoptotic cells respectively.

### Animal Studies

Pathogen-free male 5-week-old athymic nude mice (Harlan Sprague Dawley, Ind.) were housed in a specific pathogen-free environment. Food supplies and instruments were autoclaved, and all manipulations were performed in a laminar-flow hood. Animals were randomly assigned to experimental groups (n=8). Cells were inoculated subcutaneously at the right axilla of the forelimb. The size of xenografts was determined two times per week by externally measuring tumors in two dimensions. Volume was calculated by the following equation: V=(L×W²)×0.5, where L is the length and W is the width of the xenograft.

### Nomenclature of RNA Subunits:

To simplify the description in the construction of RNA complexes, uppercase and lower case letters are used to represent the right and left hand loops of the pRNA respectively, (FIG. 30A). The matched letters indicate complementarity, whereas different letters indicate non-complementary loops. For example, pRNA (A-b′) contains right hand loop A (⁵′G₄₅G₄₆A₄₇C₄₈) and left hand loop b′ (³′U₈₅G₈₄C₈₃G₈₂), which can pair with the left hand loop a′ (³′C₈₅C₈₄U₈₃G₈₂) and right hand loop B(⁵′A₄₅C₄₆G₄₇C₄₈) respectively, of pRNA(B-a′) (FIG. 30). pRNA/aptamer(CD4) denotes a pRNA chimera that harbors an aptamer that binds CD4, and pRNA/siRNA(GFP) represents a pRNA chimera that harbors a siRNA targeting green fluorescent protein (GFP). pRNA/ribozyme (survivin) represents a chimeric pRNA harboring a hammerhead ribozyme against survivin.

1. Construction of Chimeric pRNA Subunits Harboring Foreign Moieties
a. Construction of Chimeric pRNA Harboring siRNA

pRNA contains a double-stranded helical domain at 5′/3′ end and an intermolecular binding domain, which fold independently of each other. Complementary modification studies have revealed that altering the primary sequences of any nucleotide of the helical region does not affect pRNA structure and folding as long as the two strands are paired (FIG. 30)(Zhang et al. (1994), Virology, 201, 77-85). Extensive studies revealed that siRNA is a double-stranded RNA helix( Elbashir et al. (2001), Nature, 411, 494-498; Li et al. (2002), Science, 296, 1319-1321; Brummelkamp et al. (2002), 296, 550-553; Carmichael (2002), Nature, 418, 379-380). To test whether it is possible to replace the helical region in pRNA with double-stranded siRNA, a variety of chimeric pRNAs with different targets were constructed to carry siRNA connected to nucleotides #29 and 91 of the pRNA (FIG. 30), resulting in pRNA/siRNA(GFP), pRNA/siRNA(Luciferase), pRNA/siRNA(BAD), and pRNA/siRNA(Survivin).

b. Construction of Chimeric pRNA Harboring Receptor-Binding Aptamer or Ribozyme

To achieve specific delivery of therapeutic complexes, it is often necessary to incorporate a moiety that recognizes signature molecules on cell surfaces. In comparison to antibodies and phage-displayed peptides, RNA aptamer is an attractive alternative since it avoids the induction of immune responses (Goldsby et al. (2002), Antigens. Immunology, W. H. Freeman and Company, New York, pp. 57-61). Using the SELEX approach, a number of RNA aptamers were obtained that specifically recognize a particular cell surface receptor such as CD4 (Kraus et al. (1998), J Immunol, 160, 5209-5212). One CD4-binding RNA aptamer was chosen to construct chimeric pRNA/aptamer(CD4) via a mutual 5′/3′ end connection (FIG. 30D). The pRNA vector was reorganized into a circularly permuted form, with the nascent 5′ and 3′ ends relocated to residues #71 and 75, respectively, of the original pRNA sequence. The 71/75 end is located in a tightly-folded area (Hoeprich & Guo (2002), J Biol Chem, 277(23), 20794-20803) to bury and protect the ends from exonuclease degradation in vivo (Hoeprich et al. (2003), Gene Therapy, 10(15), 1258-1267). Similar rules were followed to construct the chimeric pRNA/ribozyme(survivin).

c. Construction of Chimeric pRNA Harboring Folate

Folate receptors are overexpressed in various types of tumors such as human nasopharyngeal epidermal carcinoma but are generally absent in normal adult tissues. Many therapeutic reagents such as low molecule weight drugs, antisense oligonucleotides and protein toxins have been conjugated to folate and then delivered to tumor cells (Sudimack et al. (2000), Adv Drug Deliv Rev, 41, 147-162; Lu et al. (2003), J Control Release, 91, 17-29). The same strategy was employed in this study to deliver siRNA to folate receptor-overexpressing tumor cells. The folate molecule was incorporated into the 5′ end of RNA and formed a dimer with a pRNA/siRNA chimera to achieve specific delivery (FIG. 30D). In order to increase the accessibility of the folate molecule to folate receptor on the cell surface, folate-labeled RNA was designed to be a 5′ overhang, in which nucleotides #107 to 117 of pRNA were truncated.

2. Processing of Chimeric pRNA/siRNA Complex into Double-Stranded siRNA by Dicer

To answer the question of whether the chimeric complexes could be processed into functional siRNA, chimeric pRNA/siRNA was subjected to treatment by purified recombinant Dicer, which is well-known for its function of processing long double-stranded RNA into 22 bp siRNA in vitro and in vivo. The chimeric pRNA/siRNA complex used as the substrate in this study harbored a 29-bp double-stranded siRNA connected to the pRNA inter-molecular interaction domain from nucleotides 29 to 91. Two additional uridines were used to link the siRNA to the pRNA domain to help enhance processing efficiency by increasing the AG for the folding of the loop. Purified chimeric pRNA/siRNA complex was labeled at the 5′ end with [³²P] and incubated with Dicer, and the digestion product was then analyzed by denatured PAGE/Urea. As shown in FIG. 31, digestion of pRNA/siRNA by Dicer for 30 minutes to 2 hours resulted in the production of 22-base siRNA with high efficiency. This result confirms that the chimeric pRNA/siRNA was cleaved and released the functional double-stranded siRNA located at the 5′/3′ ends.

3. Functional Assay of Chimeric pRNA Subunits by Transfection
a. pRNA/siRNA(GFP)

To test the function of pRNA/siRNA(GFP), GFP-expressing plasmid was co-transfected with various RNA chimeras into cells. Fluorescent microscopy revealed that pRNA/siRNA(GFP) effectively inhibited GFP gene expression in a dose dependent manner (FIG. 32A). In contrast, such inhibitory effects were not observed with a control construct containing site-directed mutations within siRNA sequences. Nonspecific inhibition by pRNA vector was ruled out through a control (FIG. 32B) with the vector alone (nucleotides #18-99). The effect of gene silencing on mRNA level was further demonstrated by Northern blot (FIG. 32D). In both assays, chimeric pRNA/siRNA(GFP) exhibited equivalent or superior inhibitory effects on GFP gene expression compared to chemically synthesized double-stranded siRNA(GFP) (FIGS. 32C and D).

b. pRNA/siRNA(Luciferase)

In addition to the GFP-specific chimeric siRNA, pRNA/siRNA constructs against luciferase were also constructed and tested. Two chimeric pRNA/siRNA constructs targeting either firefly luciferase or renilla luciferase were introduced into cells by transient transfection in separate experiments, and the expression levels of both luciferases were then measured simultaneously by a Dual reporter assay. When the targeted luciferase was examined, the non-targeted luciferase served as the internal control. As shown in FIG. 33A, each construct was found to suppress its target gene efficiently and specifically. No silencing of the luciferase genes occurred when mutations were introduced into the siRNA of the pRNA complexes. In addition, the silencing effect of pRNA/siRNA(firefly) was found to be more efficient than hairpin siRNA(firefly) alone (FIG. 33B).

c. pRNA/siRNA(Survivin) and pRNA/Ribozyme(Survivin) Knocked Down Anti-Apoptosis Factor Survivin and Initiated Cell Death

To evaluate the effectiveness of therapeutic RNA molecules in treating cancer, it is necessary to suppress genes involved in tumor development and progression. Survivin was chosen as a target because it inhibits apoptosis and is detected only in cancer cells and not in normal adult cells. It has been shown that the suppression of survivin induces the apoptosis of cancer cells (Grossman et al. (2001), Proc Natl Acad Sci, 98, 635-640).

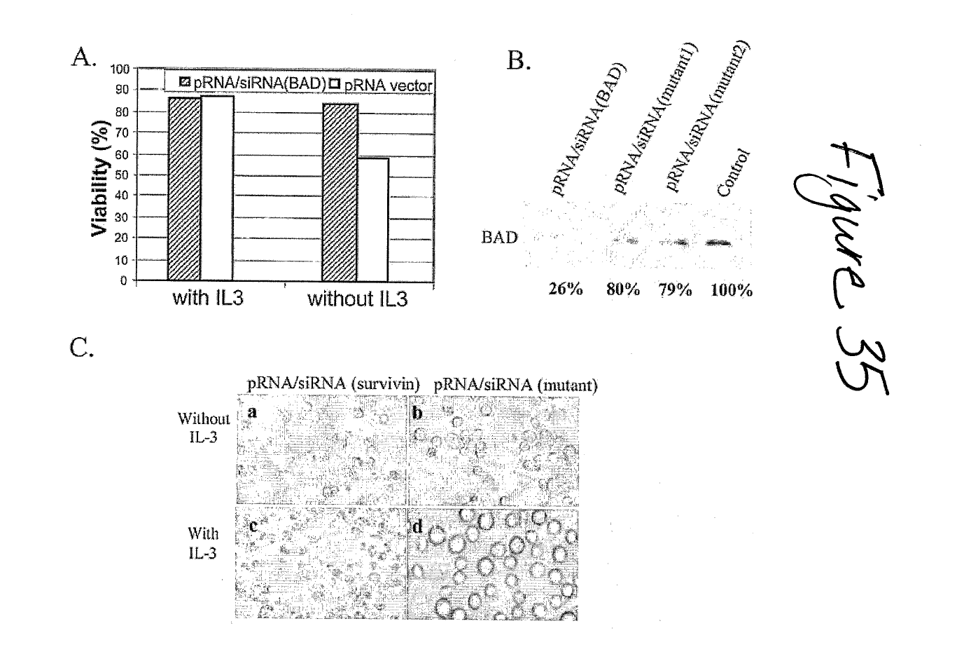

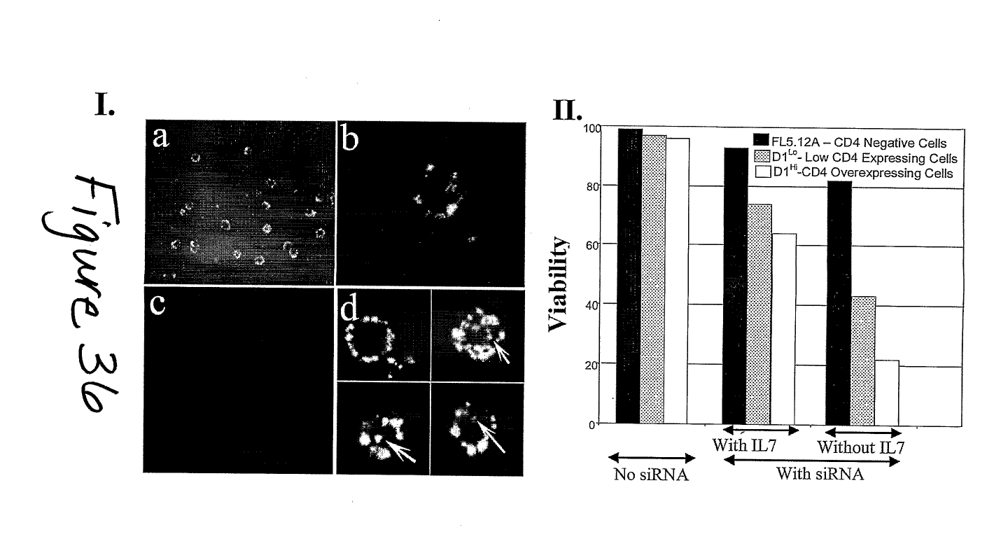

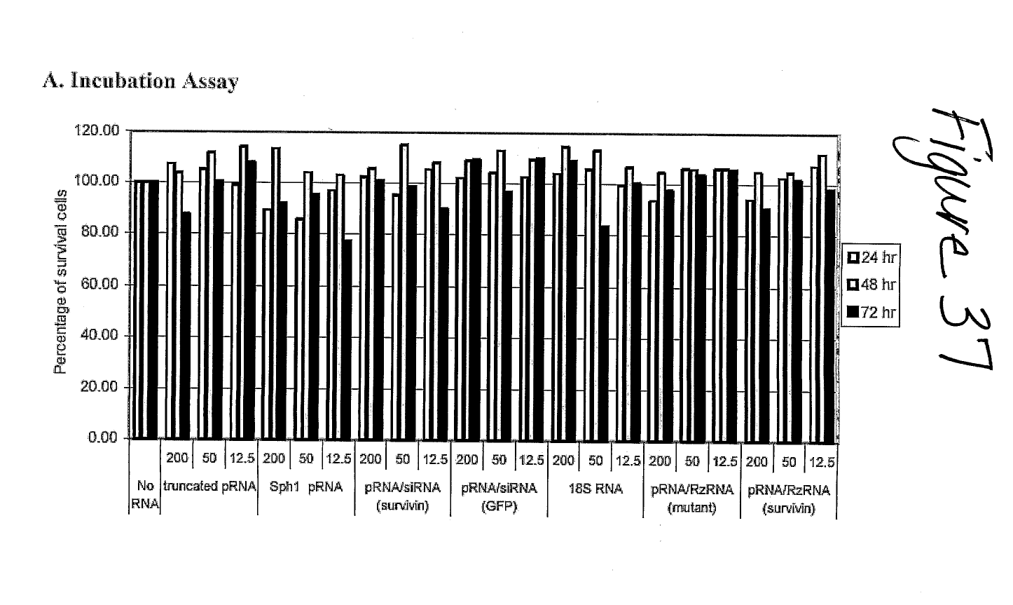

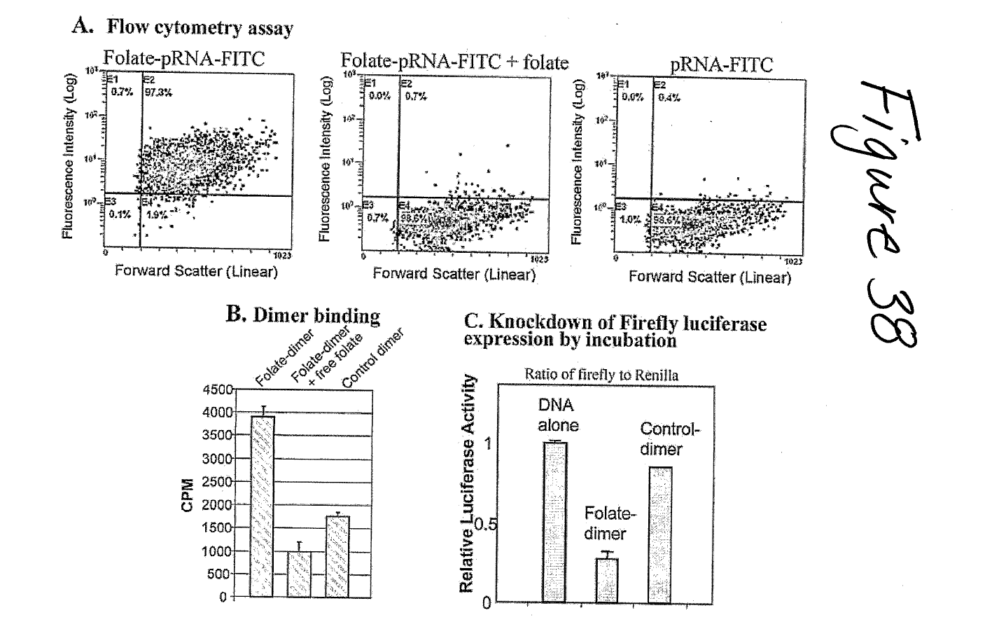

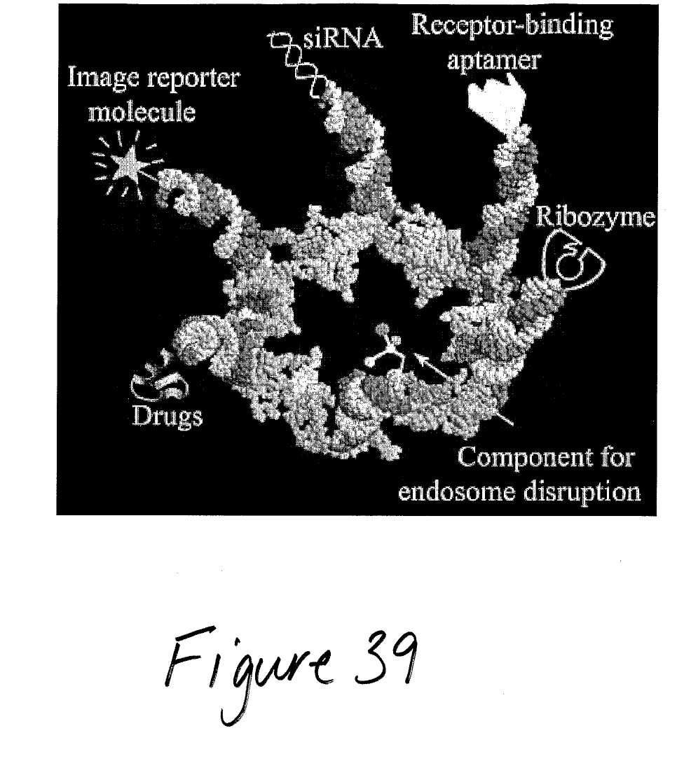

The function and specificity of pRNA/siRNA(survivin) was first examined in breast and prostate cancer cells. The apoptosis of breast cancer cells transfected with pRNA/siRNA(survivin) was assessed with annexin V-propidium iodide (PI) double-staining followed by flow cytometry analysis. As shown in FIG. 34-I-B, cells transfected with pRNA/siRNA(survivin) were shown at a much higher percentage in the lower right area representing apoptotic cells, compared to those treated with mutant chimeric siRNA or 5S RNA as negative controls (FIGS. 34-I-A, C and D). The effects of chimeric siRNA on cell survival were also evaluated by cell morphology studies in breast cancer cell line MDA-231 and prostate cancer cell line PC-3. After transfection, the majority of cells shrank and were detached from the cell culture plate, while the control pRNA/siRNA(mutant) did not induce cell death (FIG. 34-II).

The knockdown effect by pRNA/ribozyme(survivin) was also tested. When several human cancer cell lines were transfected with the PRNA/ribozyme(survivin), more than 80% of the cells were dead 24 hours after transfection (FIG. 35A). Western blot assay revealed that survivin protein expression was effectively knocked down by chimeric RNA after transfection (FIG. 35B). In contrast, when simply incubated with cancer cells, pRNA/siRNA(survivin) or pRNA/ribozyme(survivin) did not cause cell death for up to 72 hours in the absence of transfection reagent (FIG. 35C). This indicates that the chimeric pRNAs did not show non-specific cytotoxicity upon incubation. However, when introduced into cells by transfection, chimeric siRNA or ribozyme against survivin could silence the survivin gene specifically and cause cell death.

d. pRNA/siRNA (BAD) Silenced Pro-Apoptosis Factor and Prevented Cell Death

Inducing cell death by knocking down anti-apoptosis factors would be ideal in cancer therapy, but it might not be sufficient to demonstrate the specificity of the RNA delivery strategy, because cell death can potentially be caused by nonspecific effects, if any occur. To rule out the possibility of non-specific cytotoxicity, pro-apoptosis factor BAD in the BCl-2 family (Khaled et al., (2001), J Biol Chem, 276, 6453-6462) was selected as the target, since silencing pro-apoptotic factors would prevent apoptosis instead of causing cell death (Kumar et al. (2002), Science, 297, 1290-1291). Thus, the knockdown effect from siRNA could be distinguished from the nonspecific toxicity of the RNA components. To this end, IL-3 dependent pro-B cell line FL5.12A was employed. Withdrawal of IL-3 could induce the expression of BAD, leading to apoptosis of FL5.12A cells (Khaled et al. (2002), Nat Rev Immunol, 2, 817-830). pRNA/siRNA(BAD) was constructed and introduced into the pro-B cell line, and Western blot indicated that there was a significant decrease in BAD protein (FIG. 36B), while the mutant controls and pRNA vector alone resulted in only minor decreases in BAD protein compared to the cells not treated. Viability assay revealed that the transfection of pRNA/siRNA(BAD) protected FL5.12A cells from death upon IL-3 removal, and did not cause cell death in the presence of IL-3 (FIG. 36A). These results demonstrate that chimeric pRNA/siRNA can specifically silence the expression of targeted pro-apoptotic genes and prevent growth factor withdrawal-induced cell death. In contrast, cell death was induced by pRNA/siRNA(Survivin) in the presence of IL-3, and exacerbated upon IL-3 withdrawal, compared to the mutant chimeric siRNA control (FIG. 36C).

4. Assembly of pRNA Dimers Composed of Chimeric Subunits Harboring Individual Functional Subunits

Effective gene therapy requires at least two features: specific cell recognition and the silencing of specific gene(s) in cells. Construction of a RNA molecule with both functionalities would satisfy these requirements, but direct fusion or conjugation of one RNA with two or more moieties could lead to misfolding and loss of function. Construction of RNA dimer is an alternative approach for achieving these two goals.

Extensive studies of phi29 pRNA revealed that two pRNA subunits form a dimer through the interaction of the complementary left and right hand loops of pRNA monomers. The extendable 5′/3′ ends of dimer provide two separated sites to carry extra sequences without altering the secondary structure of the pRNA vectors or the inserted sequences (Shu et al. (2004), Nano Letters, 4, 1717-1724). The dimer formation mechanism was therefore utilized to construct the RNA complex to deliver functional moieties for 1) specific recognition mediated by receptor-binding RNA aptamer or folate; and 2) regulation of cell functions (growth, death, physiology, etc.) mediated by siRNA or ribozyme.

The sequence responsible for intermolecular pRNA/pRNA interaction is located between residues 23-97 (Chen et al. (1999),RNA, 5, 805-818). Chimeric monomer subunits were intentionally designed to possess either A-b′ or B-a′ to match with each other. When pRNA A-b′ and B-a′ were mixed in a 1:1 molar ratio in the presence of 10 mM Mg²⁺, pRNA dimers were produced with high efficiency, as confirmed by gel electrophoresis and cryo-AFM imaging (FIG. 30C). In AFM images, the monomer exhibits a checkmark shape and the dimer is twice as large as the monomer. The data indicated that dimers were generated from monomer subunits despite the replacement of the 5′/3′ helix with double-stranded siRNA or connection of the 5′/3′ end to an aptamer or ribozyme. The correct folding of pRNA chimera was also confirmed by phi29 in vitro assembly inhibition assay as previously reported (Guo et al. (1987), Science, 236, 690-694; Guo et al. (1998), Mol Cell, 2, 149-155).

5. Functional Assay of Chimeric pRNA Dimer by Incubation
a. Binding Assay of Chimeric pRNA Containing CD4 Aptamer

CD4 is a receptor displayed on the surface of certain T lymphocytes. A CD4-overexpressing T cell line (referred to as CD4^{Hi}) was developed from a murine IL-7-dependent proT cell line D1 (referred to as CD4^{Low}) (Kim et al. (2003), J Immunol Methods, 274, 177-184) that normally expresses undetectable levels of endogenous CD4. Incubation of CD4^{Hi} cells with RNA dimer composed of pRNA(A-b′)/aptamer(CD4) and pRNA(B-a′)/FITC revealed strong and specific binding (FIG. 37I), since binding was not detected in FL5.12A cells with no CD4 expression (FIG. 37-I-c) or FITC-dimer without aptamer(CD4) (data not shown).

b. Incubation with Dimer Harboring both Aptamer(CD4) and siRNA(Survivin)

The strategy of RNA dimer-mediated gene delivery is that the receptor-binding moiety mediates cell recognition and subsequent internalization, and the siRNA is then released to down-regulate specific genes. Dimers containing both pRNA/siRNA(survivin) and pRNA/aptamer(CD4) were incubated with cells with different levels of CD4 expression. The CD4^{Hi} cells responded most strongly to the pRNA dimeric complex, which showed more than 30% reduction in cell viability in the presence of IL-7. Upon IL-7 removal, both D1 CD4^{Low} and CD^{Hi} exhibited severe cell death compared to CD4-negative FL5.12A cells. The level of cell death was correlated with the expression level of CD4. These results suggest that CD4-mediated entry of survivin siRNA led to the suppression of cell viability and that such effects were dependent upon the level of CD4 molecules on the cell surface.

c. Binding Assay of Chimeric pRNA Conjugated with Folate

Specific cell binding of pRNA/folate monomer was demonstrated by flow cytometry (FIG. 38A). When human nasopharyngeal epidermal carcinoma I(B cells with endogenous over-expressed folate receptors were incubated with FITC-pRNA/folate, 97.3% of the cells were FITC-positive. Adding free folate as a blocking reagent to the incubation buffer decreased the FITC-positive cells to only 0.7% (FIG. 38A), suggesting that the binding of pRNA to cells is specific and dependent upon folate receptor. FITC-labeled pRNA without folate conjugation did not exhibit binding.

The possibility of using folate to carry dimeric siRNA chimera was also demonstrated through the use of radiolabeled RNA. First, the RNA dimer was generated by mixing equal amounts of pRNA(A-b′) and [H³]-pRNA(B-a′) in the presence of Mg²⁺. The folate-dimer showed much stronger binding compared to the control dimer without folate labeling (FIG. 38B). When free folate was included as a blocking reagent, the binding of folate-labeled heterodimer RNA to cells diminished.

d. Functional Assay for pRNA/siRNA(Firefly or Renilla) after Delivery to Cells by Incubation

After the specific knockdown of chimeric siRNA against luciferase (Result 3-b) and the specific binding of folate-labeled dimer (Result 5-c) were demonstrated, the gene silencing effects by incubation were farther investigated. To determine whether folate-mediated targeting of chimeric siRNA to folate receptor-overexpressing cells can lead to the entry of the RNA complex and knockdown of a specific gene, dimers composed of pRNA/siRNA(firefly) and pRNA/folate were incubated with folate-receptor positive KB cells, to allow the binding and entry of RNA mediated by folate. Dual assay revealed a dramatic decrease in firefly luciferase expression after incubation (FIG. 38C). A control RNA without folate labeling, on the contrary, did not significantly interfere with target protein expression. The specificity of pRNA/siRNA(firefly) in dimers was demonstrated when renilla luciferase was used as an internal control.

6. Animal Trials to Demonstrate Specific Suppression of Tumorigenicity of Cancer Cells by Ex vivo Delivery of Chimeric siRNA Against Survivin

Animal trials were conducted to test the specificity by ex vivo delivery using a dimer containing both pRNA(A-b′)/folate and pRNA(B-a′)/siRNA(survivin). The potential of this RNA dimer to suppress tumor formation was tested in athymic nude mice. Nasopharyngeal epidermal carcinoma (KB) cells were incubated with dimeric RNA with or without the blocking reagent, free folate, before being introduced into the nude mice by axilla injection. The mice receiving cells alone developed tumors within 3 weeks, while none of the mice receiving cells pretreated with the dimers with pRNA(A-b′)/folate and pRNA(B-a′)/siRNA(survivin) developed tumors (Table 3). The inhibition of tumor formation is specific since the control dimer RNA without folate conjugation used in control mice groups did not affect tumor development.

TABLE 3
Animal Trial in Epidermal Cancer Therapy^{a}

| | | No. of cancerous animals/ |
|---|---|---|
| Treatment group | | no. of animals tested. |
| 1. | No pRNA | 4/8 |
| 2. | Dimer [folate-pRNA(B-a′) + | 0/7 |
| | pRNA/siRNA(survivin) (A-b′)] | |
| 3. | Dimer [pRNA(B-a′) + pRNA/mutant- | 7/8 |
| | siRNA(A-b′] | |
| 4. | Dimer [pRNA(B-a′) + | 6/8 |
| | pRNA/siRNA(survivin)(A-b′)] | |

^{a}KB cells were maintained in folate-free medium RPMI 1640. Cells were preincubated with pRNA complex for 3 hours before being used for animal injection. After rinsing twice with PBS containing 10 mM MgCl₂ cells were collected into a centrifuge tube. Each mouse was incoulated iwth 2.5 × 10³ cells in 0.1 mL of medium. Shown are the results of in vivo testing of mice receiving tumor xenografts along with the chimeric pRNA complex. One mouse in group 2 produced a plaque within 1 week, much earlier than any of the other mice in any group, and therefore given these special circumstances it was treated as an outlier and there are seven mice recorded for group 2 instead eight.

### Discussion

Phi29 pRNA has a tendency to form dimers, which are the building blocks of hexamers (FIG. 39), as a result of the interaction of interlocking loops of each pRNA. This manuscript demonstrated the production of dimers to deliver therapeutic RNA to specific cells. In the future, chimeric hexamers could also be assembled via hand-in-hand interaction. Thus, since there are six chimeric pRNAs in the hexamer, there would be six positions available to carry molecules for cell recognition, therapy, and detection. Besides the receptor-binding aptamers, siRNA, ribozyme and folate reported here, other materials such as fluorescent dyes, heavy metal, quantum dots, fluorescent beads or radioisotopes can also be conjugated for the detection of cancer signatures at different stages of development. The reported methods for conjugating folate and FITC could be used for the conjugation of chemical drugs, and endosome-disrupting chemicals could be added to promote the release of siRNA from the endosome after delivery to improve therapeutic efficacy (FIG. 39). Nucleotide derivatives such as 2′-F-2′ deoxy CTP, 2′-F-2′ deoxy UTP or Spiegelmer will be incorporated into the RNA to produce stable in vitro RNA transcripts that are resistant to RNase digestion (Soutschek et al. (2004), Nature, 432, 173-178).

This polyvalent RNA complex can also potentially be used for treating chronic viral infections such as those caused by HIV and hepatitis B virus through targeting at the specific virus-glycoproteins incorporated on the infected cell surface. It is well-established in the scientific community that RNAs do not induce a detectable immune response except when complexed with proteins (Madaio et al. (1984), J Immunol, 132, 872-876; Goldsby et al. (2002), Antigens. Immunology, W. H. Freeman and Company, New York, pp. 57-61). The use of RNA as a delivery vehicle could avoid the problems of immune response and the rejection of protein vectors after repeated long-term drug administration. The use of such a 30-40 nanometer RNA complex would provide a longer turnover time in the body than other small molecules would offer.

### Example 13

### Construction of Folate-Conjugated Phage Phi29 Motor pRNA for Delivery of Chimeric siRNA to Nasopharyngeal Carcinoma Cells

Nasopharyngeal carcinoma is a poorly differentiated upper respiratory tract cancer that highly expresses hFR (human folate receptors). Binding of folate to hFR triggers endocytosis. The folate was conjugated into AMP by 1,6-hexanediamine linkages. After reverse HPLC to reach 93% purity, the folate-AMP, which can only be used for transcription initiation but not for chain extension, was incorporated into the 5′-end of phi29 motor pRNA. A 16:1 ratio of folate-AMP to ATP in transcription resulted in more than 60% of the pRNA containing folate. A pRNA with a 5′-overhang is needed to enhance the accessibility of the 5′ folate for specific receptor binding. Utilizing the engineered left/right interlocking loops, polyvalent dimeric pRNA nanoparticles were constructed using RNA nanotechnology to carry folate, a detection marker, and siRNA targeting at an anti-apoptosis factor. The chimeric pRNAs were processed into ds-siRNA by Dicer. Incubation of nasopharyngeal epidermal carcinoma (KB) cells with the dimer resulted in its entry into cancer cells and subsequent silencing of the target gene. Such a protein-free RNA nano-particle with low antigenicity has a potential for repeated long-term administration for nasopharyngeal carcinoma since the effectiveness and specificity were confirmed by ex vivo delivery in the animal trial.

### Introduction

A 117-nt bacteriophage phi29-encoded RNA (pRNA) has been found to play a novel and essential role in DNA packaging(Guo et al., Science 1987;236:690-694). pRNA forms dimers, trimers and, ultimately, hexamers through hand-in-hand interaction of the right and left interlocking loops (Guo, Prog Nucl Acid Res Moe Biol 2002; 72:415-472; Shu et al., Nano Letters 2004; 4:1717-1724). The structural features of pRNA, which have been studied extensively, allow for easy manipulation and permit the conversion of pRNA into a gene targeting and delivery vehicle. The pRNA molecule contains two independent folding domains with distinct functions (Guo, Prog Nucl Acid Res Mol Biol 2002; 72:415-472; Zhang et al., Virology 1994; 201:77-85). Replacement or insertion of nucleotides preceding residue #23 or following residue #97 does not interfere with the formation of dimers as long as the strands are paired (Chen et al., RNA 1999; 5:805-818). Therefore the 5′/3′ proximate double-stranded helical region (Zhang et al.m RNA 1995; 1:1041-1050) of pRNA can be redesigned to carry additional sequences without altering its secondary structure or inter-molecular interactions (Hoeprich et al., Gene Therapy 2003; 10(15):1258-1267; Shu et al., J Nanosci and Nanotech (JNN) 2003; 3:295-302).

Being a poorly differentiated carcinoma of the human upper respiratory tract, nasopharylngeal carcinoma has human folate receptors (hFR) that are highly expressed in KB cells. Endocytosis of the ligand/receptor complex mediated by the binding of folate to hFR has been well-studied, and macromolecules conjugated to folate have been successfully recognized by folate receptors and internalized into cells (Lee et al., J Biol Chem 1994; 269:3198-3204; Mathias et al., J Nucl Med 1996; 37:1003-1008; Benns et al., J Drug Target 2001; 9:123-139). Recently, we found that phi29 pRNA can be used as a carrier for the construction of RNA nanoparticles to deliver therapeutic RNAs such as siRNAs and/or ribozymes to specific cancer cells. (Shu et al., Nano Letters 2004; 4:1717-1724; Guo et al., Human Gene Therapy 2005; 16:1097-1109 [Example 12]; Khaled et al., Nano Letters 2005; 5:1797-1808 [Example 11]) In this report, we described the methods for the synthesis and purification of folate-AMP, the procedure for the construction of folate-pRNA, and the test of the purity and function of the folate-containing products. The potential of siRNA in gene therapy mediated by folate was also investigated in animal trials.

### Materials and Methods

Preparation of RNA. RNA preparation and the characterization of dimer were described in our previous publications (Zhang et al., Virology 1994; 201:77-85; Guo et al., Mol Cell 1998; 2:149-155). Briefly, RNAs were prepared by in vitro transcription using T7-MegaShortscript Kit purchased from Ambion. DNA templates with T7 polymerase were used in the presence of 7.5 mM ATP, 7.5 mM GTP, 7.5 mM UTP, and 7.5 mM CTP. 1 μl [α-³²P] ATP or [³H] UTP was included for radioactive labeling of RNA. Transcription products were purified by 8% PAGE/Urea and eluted with 0.5 M sodium acetate, 0.1 mM EDTA and 0.1% SDS. RNAs were ethanol precipitated and resuspended in depc-treated water. To label the 5′-end of RNA with folate, both 4 mM folate-AMP and 0.25 mM ATP were included in a transcription reaction, together with 1 mM UTP, CTP, and GTP.

Synthesis and purification of folate-AMP. The conjugation of folate with adenosine 5′-monophosphate (AMP) was achieved by introducing a folate moiety to AMP through the linker molecule 1,6-hexanediamine (HDA), based on similar conjugation chemistry as published (Huang et al., RNA 2003; 9:1562-1570). 5′ Folate-AMP was then purified to 93% purity by semi-preparative reverse phase HPLC. After lyophilization, the compound was dissolved in water. Its concentration was determined by absorbance at 350 nm, with a molar extinction coefficient of ε₃₅₀=8,000 M⁻¹·cm⁻¹. The folate-AMP was then used directly for the preparation of folate-conjugated RNA under the T7 φ2.5 promoter under the published conditions.

Cell culture. KB cells were maintained in a folate-free RPMI1640 medium (Gibco) supplemented with 10% FBS and penicillin/streptomycin in a 5% CO₂ incubator. The serum provided a normal complement of endogenous folate for cell growth.

Flow cytometry analysis. 4×10⁵ KB cells were seeded into a 6-well plate and grown for 24 h. After being rinsed twice with PBS, the cells were incubated with 100 nM Folate-FITC for 20 minutes at room temperature, with or without the presence of blocking reagent. 166 μM free folate or folate-AMP was included as blocking reagents. Cells were then washed and harvested in PBS and analyzed by flow cytometry.

Binding offolate RNA to KB cells. One micromolar RNA was added to a suspension of 10⁷ cells in 0.5 ml of medium in the presence of 10 mM Mg²⁺ and incubated at 37° C. for 30 minutes. Cells were then washed twice with RPMI1640 medium, and the radioactivity of cells was measured by a liquid scintillation counter.

Dual-Luciferase assays. Gene silencing assay by transfection was performed by co-transfecting various chimeric siRNA into mouse fibroblast PA3 17-PAR cells with both pGL3 plasmid encoding firefly luciferase and pRL-TI plasmid encoding Renilla luciferase. Both luciferase activities were measured by Dual-Luciferase Reporter Assay System (Promega).

For the incubation assay, the folate-dimer was prepared by mixing folate-pRNA (7-106) B-a′ and pRNA/siRNA (Firefly luciferase) A-b′ with 10 mM Mg²⁺. KB cells were seeded in a 6-well plate in folate-free medium. After being washed by PBS-supplied MgCl₂, the pre-mixed dimer RNA (1.75 μM) was then added to cells and incubated for 3 h at 37° C. RNase inhibitor SUPERRNaseIN (1 unit/ul) (Ambion) was added into the binding buffer. After incubation, free RNA was washed off, and pGL3 and pRL-TK plasmids were introduced into cells using Lipofectamine 2000 (Promega). Luciferase activities were measured the next day.

Chimeric pRNA/siRNA processing. The method in the publication in Nature for demonstrating the processing of the double-stranded RNA into siRNA (Bernstein et al., Nature 2001; 409:363-366) was followed exactly in this study. The purified recombinant Dicer was purchased from Gene Therapy Systems.

In vivo animal studies. Five-week-old male athymic nude mice (Harlan Sprague Dawley) were housed in a pathogen-free environment. Animals were randomly assigned to experimental groups (n=8). Cells were maintained in antibiotic-free medium for one week before injection. On the day of injection, cells were rinsed with PBS twice and incubated with the dimeric pRNA complex at 37° C. for 3 hours before being collected by trypsin digestion. 2.5×10⁵ cells in 0.1 ml of media were used for the injection of each mouse. Cells were inoculated subcutaneously in the right axilla of the forelimb. Once xenografts were visible, their size was determined two times per week by externally measuring tumors in two dimensions. Volume was calculated by the following equation: V=(L×W²)×0.5, where L is the length and W is the width of the xenograft.

### Results

1. Characterization of Folate-AMP

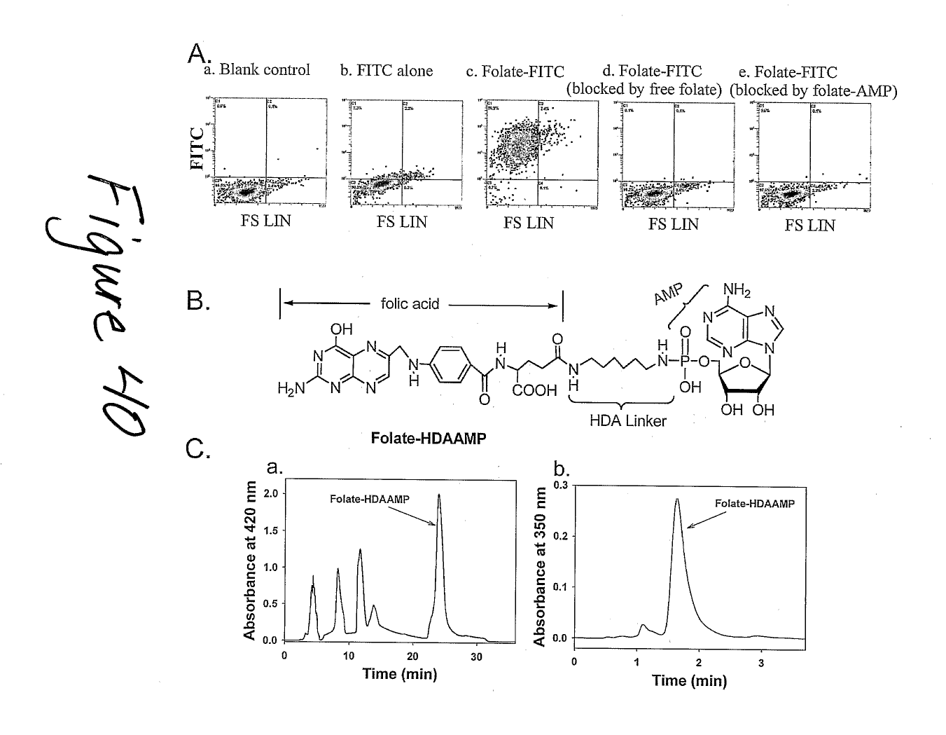

A folate-AMP complex (FIG. 40) was synthesized by conjugating folic acid with adenosine 5′-monophosphate (AMP) through the linker molecule 1,6-hexanediamine (HDA) by established chemistry (Huang et al., Biochemistry 2000; 39:15548-15555). The complex was purified by semi-preparative reverse phase HPLC (FIG. 40C). The purity of the folate-AMP complex was determined by both reverse phase HPLC and thin-layer chromatography. The compound, exhibiting 93% purity (FIG. 40C), was used for the synthesis of folate-pRNA as discussed below.

To determine whether folate-AMP is able to bind to the folate receptor on the cell surface, the capability of folate-AMP to compete with folate-FITC for binding to human nasopharyngeal carcinoma KB cells was assessed by flow cytometry. 97% of KB cells, which are folate-receptor positive, exhibited strong binding by folate-FITC (FIG. 40A). However, only 0.1% of cells were detected to contain folate-FITC when KB cells were pre-incubated with folate-AMP, which served as a competitor with folate-FITC (100 nM) for folate receptor binding (FIG. 40A). Similar blockage was observed when free folate was used. These results indicate that the folate moiety incorporated into AMP retains a high binding capacity for the folate receptor. 100nM FITC was also included as a negative control to exclude the non-specific binding between FITC and KB cells.

2. Synthesis of Folate pRNA

Previously, specific 5′-end modifications of pRNA had been achieved through use of T7 RNA polymerase with GMPS for transcription initiation (Garver et al., RNA 1997; 3:1068-1079) The main limitation in such an application is that only RNA starting with a “G” can be applied (Seelig et al., Bioconjug Chem 1999; 10:371-378). Currently, through the use of the T7 class II promoter, (Huang, Nucleic Acids Res 2003; 31:e8; Pan et al., Science 1991; 254:1361-1364) adenosine can also serve for the initiation of transcription, and so we have been able to incorporate AMP derivatives into the 5′-end of the pRNA or circular permutated pRNA (cpRNA) in one-step labeling. AMP derivatives such as folate-AMP can only be used for initiation but not for chain extension, thus ensuring that labeling occurs only at the 5′-end.

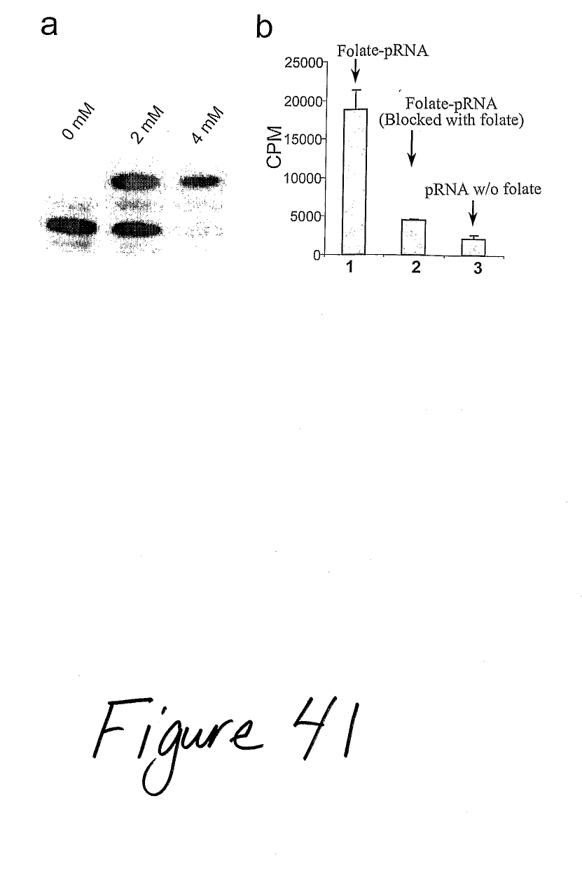

In vitro transcription of pRNA was performed in the presence of both folate-AMP and ATP, together with CTP, UTP and GTP. Different molar ratios of AMP:ATP were examined in transcription reactions. Our results show that 16:1 is the best ratio of folate-AMP to ATP in considering both total yield of the RNA production and the percentage of pRNA carrying the folate labeling. The RNA labeled with folate had a slower migration rate compared to non-labeled RNA on denaturant Urea/PAGE (FIG. 41A). Results indicate that under the optimal transcription conditions, more than 60% of the pRNA contains a folate moiety, as estimated from the gel (FIG. 41A).

3. Binding of Folate-pRNA to Nasopharyngeal Carcinoma Cells.

The size of a motor pRNA monomer was determined to be 11 nm (Hoeprich et al., J Biol Chem 2002; 277(23):20794-20803). The binding of this nanometer-scale particle with folate labeling was examined. A pRNA (7-106) with a 5′-overhang was constructed to enhance the accessibility of the 5′ folate for receptor binding (FIG. 41B). [³H]UTP was included in the transcription reaction to uniformly label the RNA. [³H]-folate-RNA exhibited strong binding to KB cells compared to the RNA without folate (FIG. 41B). Since the binding was blocked by free folate, the specificity in binding mediated by the folate receptor was demonstrated. The recessive blunt or overhanging of the 3′ end noticeably reduced the binding efficiency of the folate-pRNA to the receptor.

4. Binding of Nanoparticles Containing both Folate-pRNA and siRNA Chimera to Nasopharyngeal Carcinoma Cells

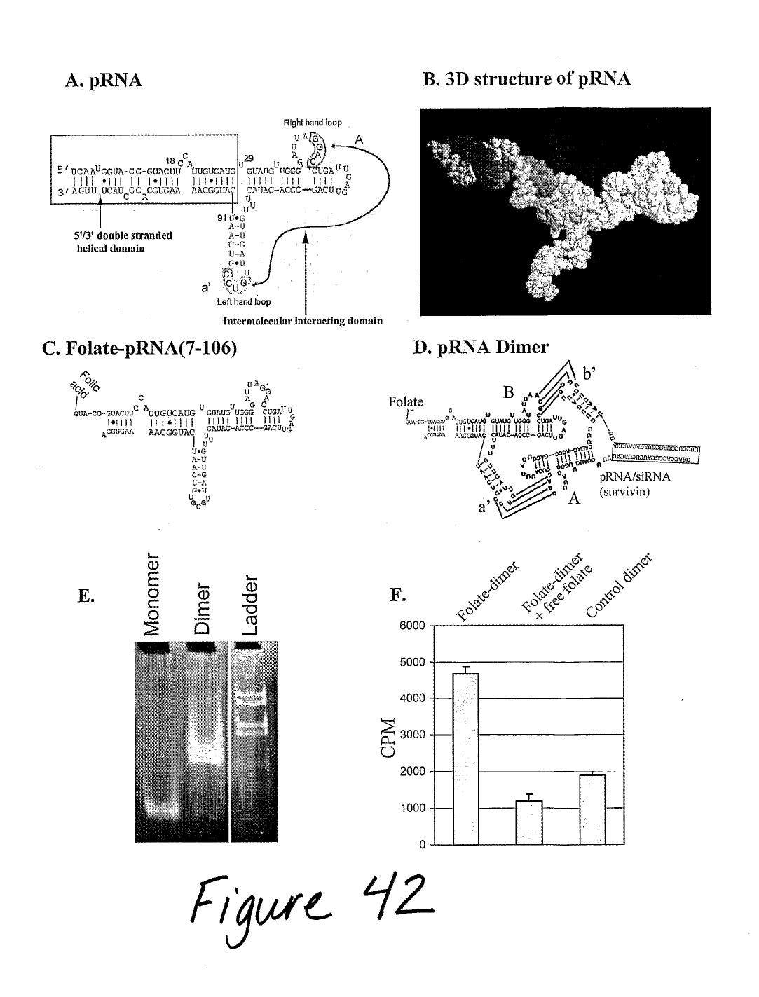

Phi29 pRNA contains two interlocking loops that can be manipulated to produce desired stable dimers approximately 20 nm in size (Hoeprich et al., J Biol Chem 2002; 277(23):20794-20803; Chen et al., J Biol Chem 2000; 275(23):17510-17516). For example, pRNA (A-b′) contains a right hand loop A (^{5′}G₄₅G₄₆A₄₇C₄₈) and a left hand loop b′ (^{3′}U₈₅G₈₄C₈₃G₈₂), which together can pair with the left hand loop a′(³′C₈₅C₈₄U₈₃G₈₂) and the right hand loop B(⁵′A₄₅C₄₆G₄₇C₄₈) of pRNA (B-a′), respectively (FIG. 42). A chimeric pRNA/siRNA monomer was constructed by replacing the double-stranded helical region of pRNA with siRNA sequences without affecting the gene silencing function and the pRNA secondary structure. (Guo et al., Human Gene Therapy 2005; 16:1097-1109 [Example 12]; Khaled et al., Nano Letters 2005; 5:1797-1808 [Example 11]). The deliverable folate containing nanoparticles was conjugated by mixing equal molar amounts of folate-pRNA (B-a′) with a chimeric pRNA/siRNA (A-b′) via the interaction of the interlocking loops (FIG. 42). The formation of the dimer was demonstrated by native-PAGE, cryo-AFM (FIG. 42), and ultracentrifugation. To assess the binding capacity of such a pRNA dimer to a folate receptor, an RNA complex composed of [³H]-(A-b′) pRNA and unlabeled folate-(B-a′) pRNA was incubated with KB cells. The folate-labeled RNA dimer showed much stronger binding compared to the control RNA dimer without folate labeling (FIG. 42). The binding specificity was demonstrated by blockage with free folate.

5. Entry of Nanoparticles Containing both Folic-pRNA and siRNA Chimera to Nasopharyngeal Carcinoma Cells

To determine whether the folate moiety on the chimeric pRNA dimer could mediate the entry of the complex into KB cells, and siRNA was conjugated to the complex for specific gene silencing. 1.75 μM RNA dimer, containing both folate and siRNA against firefly luciferase, was incubated with, rather than transfected into, KB cells. The expression level of firefly luciferase in cells treated with folate-RNA dimer decreased to 30% of cells without RNA treatment. In contrast, cells treated with a control folate-free RNA dimer retained 85% of luciferase gene expression. These results suggest that specific knockdown of the firefly luciferase gene was achieved by folate receptor-mediated internalization of chimeric pRNA dimer in the absence of transfection reagents.

6. Processing of the pRNA Chimera and the Complex of Dimer pRNA Chimera into siRNA by Dicer

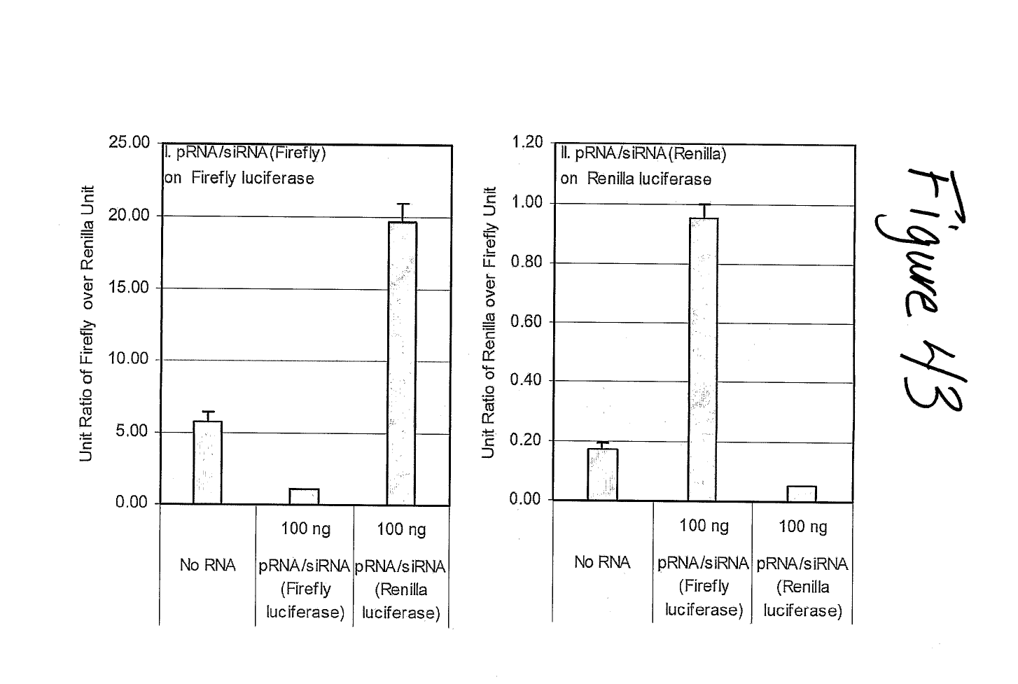

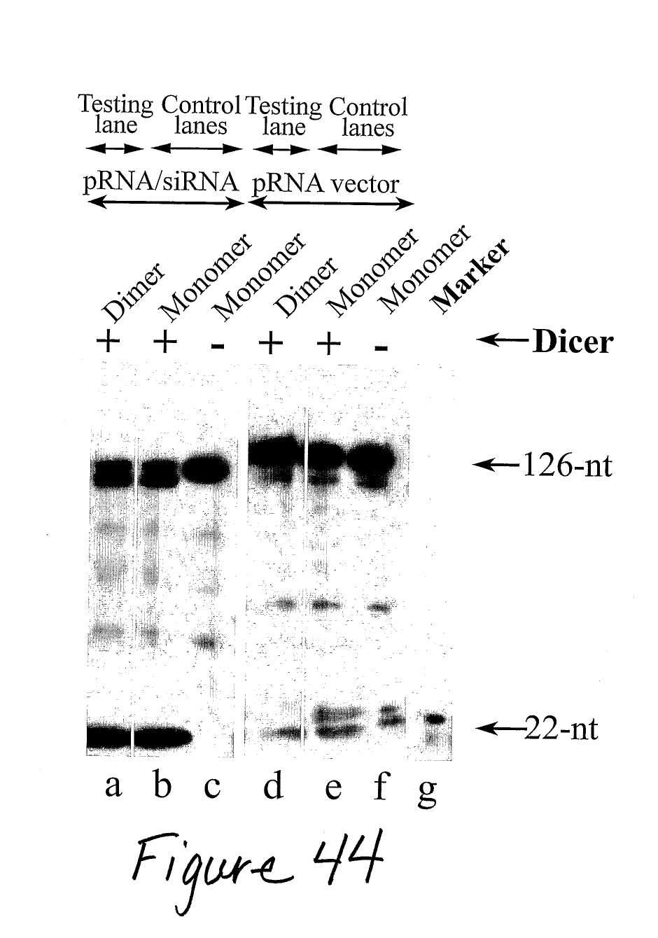

Our previous work has shown that exogenous sequences added to the 5′/3′ end of pRNA such as siRNA or hammerhead ribozyme retained biological function in cells. (Hoeprich et al., Gene Therapy 2003; 10(15): 1258-1267; Guo et al., Human Gene Therapy 2005; 16:1097-1109 [Example 12]; Khaled et al., Nano Letters 2005; 5:1797-1808 [Example 11]). In this study, a chimeric pRNA/siRNA targeting firefly or renilla luciferase was constructed, and the silencing efficiency was tested by transient transfection. The chimeric siRNA construct suppressed its target gene specifically and efficiently as demonstrated by a Dual reporter assay, in which the expression levels of two different luciferases were measured in the presence of a chimeric pRNA harboring the siRNA targeting one of the luciferases. The non-targeted luciferase served as the internal control. No silencing of the luciferase gene occurred when mutation was introduced into the siRNA of the pRNA complex (FIG. 43).

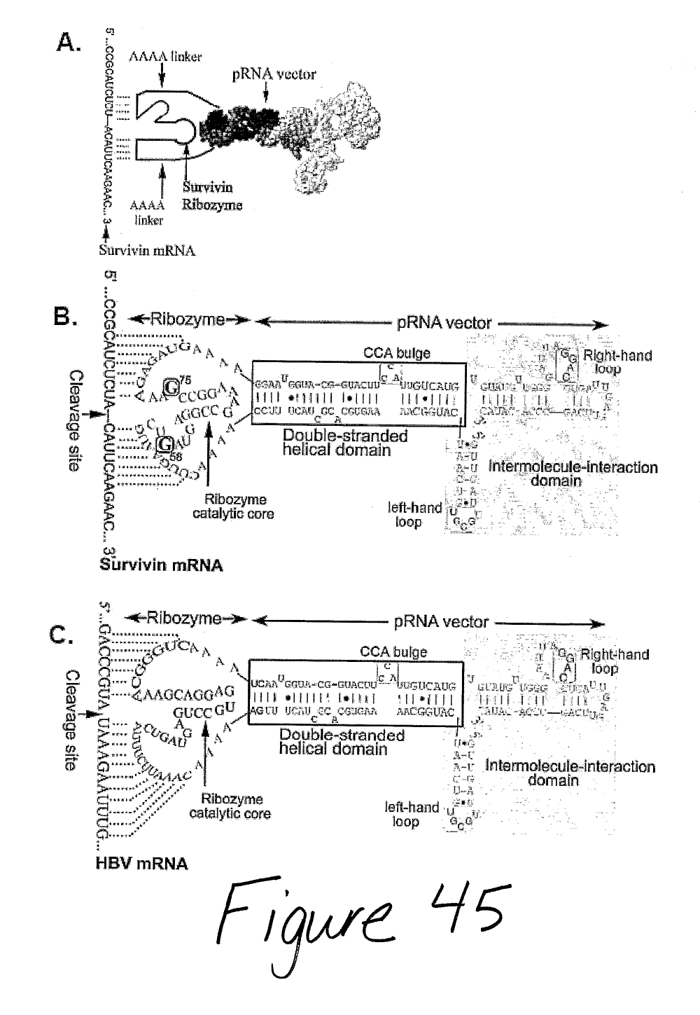

To determine whether the knockdown effects shown above were siRNA-specific, chimeric pRNA/siRNA monomers or dimers were treated with cell lysate or recombinant purified Dicer (FIG. 44), which is known for its unique function in processing long double-stranded RNA into 22-bp siRNA (Carmell et al., Nat Struct Mol Biol 2004; 11:214-218)

Incubation of the 5′-[γ³²P]-pRNA/siRNA complex with cell lysate resulted in the processing of the chimeric RNA complex into a 29-base double-stranded siRNA (not shown). Such processing is expected since we intentionally introduced two uridines at the three-way junction to increase the free energy for the folding of the junction area into a single-stranded loop. Incubation of the pRNA/siRNA complex, harboring a 29-base double-stranded siRNA, with purified Dicer resulted in the processing of the chimeric RNA complex into a 22-base double-stranded siRNA (FIG. 44), as revealed by denaturant urea PAGE. These results suggest that the chimeric pRNA/siRNA complex specifically cleaved to release the functional double-stranded siRNA at the 5′/3′ ends, and that the function of gene silencing resulted from the siRNA after the RNA complex was delivered into the cells. The dimer formation of chimeric pRNA/siRNA did not interfere with the processing and release of functional siRNA.

7. Animal Trials Demonstrate Specific Suppression of Tumorigenicity of Cancer Cells by Ex vivo Delivery of Chimeric siRNA Against Survivin

Animal trials were conducted to test the specificity by ex vivo delivery using a dimer containing both pRNA(A-b′)/folate and pRNA(B-a′)/siRNA (survivin). The potential of this RNA dimer to suppress tumor formation was tested in athymic nude mice. KB cells were incubated with various dimeric RNA samples before being introduced into the nude mice by axilla injection. Four out of the eight mice receiving cells alone developed tumors within 3 weeks; six out of the eight mice receiving dimer [pRNA (B-a′)+pRNA/siRNA (survivin) (A-b′)] without folate labeling developed tumors, and seven out of the eight mice receiving dimer [pRNA (B-a′)+pRNA/mutant siRNA (A-b′)] developed tumors. Each group of these mice exhibited an average tumor size in excess of 100 mm³ within forty-one days of injection. In contrast, only one of the eight mice receiving cells pretreated with dimers with pRNA(A-b′)/folate and pRNA (B-a′)/siRNA (survivin) developed a tumor. This single mouse produced a plaque within a week, much earlier than the mice in any of the other groups, and therefore, given these special circumstances, it was treated as an outlier. The inhibition of tumor formation is specific since the control dimer RNA without folate conjugation used in control mouse groups did not affect tumor development.

### Discussion

The goal of this work was to construct folate-conjugated phi29 pRNA for delivery of chimeric siRNA to nasopharyngeal carcinoma cells via folate receptor. Folate-labeling was achieved by utilizing folate-AMP as an initiator of RNA transcription with a T711 promoter; although folate-AMP might have some inhibitory effects on transcription yields when used at high concentrations. Phage phi29 pRNA was used as a vector to carry siRNA sequences. In addition, both breast cancer cells and ovary cancer cells were specifically stained by folate-FITC, indicating that this delivery method can also apply to at least two additional kinds of cancer cells.

The pRNA/siRNA were processed by Dicer and released double-stranded siRNA duplex, which led to specific suppression of gene expression. A stable pRNA dimer was generated by mixing two pRNAs, one of which carried folate labeling while the other carried siRNA sequences. When this RNA complex was mixed with KB cells in the absence of transfection reactions, the folate moiety was shown to (1) mediate the binding of dimeric complex and (2) mediate the knockdown of targeted luciferase gene expression. Furthermore, the suppression of tumor growth was achieved in mouse trials by incubating the folate-siRNA complex against the survivin gene, which plays an important role in tumor development.

Phi29 pRNA forms dimers as a result of the interaction of interlocking loops of each pRNA. In the future, chimeric trimers or even hexamers will be assembled by manipulating the sequence of interlocking loops. The polyvalent nature of pRNA will facilitate carrying multiple components with various functions including cell recognition, detection, endosome escape and gene suppression. Nucleotide derivatives will be utilized to produce stable RNase-resistant RNA to improve the silencing efficiency (Soutschek et al. Therapeutic silencing of an endogenous gene by systemic administration of modified siRNAs. Nature 2004; 432:173-178). This polyvalent RNA complex could also be potentially useful in treating chronic viral infectious diseases caused by HIV or HBV by targeting the specific virus-glycoproteins present on the infected cell surface.

One advantage of this strategy is that gene silencing can be achieved simply by mixing an RNA complex with cancer cells without the aid of transfection reagents derived from cationic lipids or CaCl₂. More importantly, since cancer cells express a variety of signature receptors at different stages of development, some endocytable receptors could be used as carriers to mediate the entry of therapeutic reagents labeled with the receptor ligand. Another advantage in using RNA as a delivery vehicle is the ability to avoid the problem of immune response and the rejection of protein vectors after repeated long-term drug administration.

### Example 14

### Bacteriophage Phi29 pRNA/Ribozyme Chimera Targeting Survivin for Induction of Apoptosis in Various Cancer Cells

As reported herein, bacteriophage phi29 motor pRNA has been engineered to build multivalent RNA nanoparticles for specific cell delivery of therapeutic RNAs via interlocking loops of chimeric pRNA that carries ligands, aptamer or siRNA. In this example we report the construction of a chimeric ribozyme as an additional subunit for the assembly of deliverable RNA nanoparticles. The gene silencing effects of this chimera was demonstrated in mRNA and protein level. The chimera caused the cell death of various human cancer cell lines, including breast cancer, prostate cancer, cervical cancer, nasopharyngeal cancer, and lung cancer, without causing significant level of non-specific cytotoxicity. The chimera retained its competency to form a deliverable multi-subunit complex.

To overcome the barriers in therapeutic ribozyme application concerning low in vivo efficiency caused by degradation, misfolding after fusion to carrier, and aberrant cell trafficking, the ribozyme was connected to the tightly folded 5′/3′ nascent ends of the circularly permuted pRNA to ensure appropriate folding and to enhance the stability.

### Introduction

pRNA or packaging RNA is a 117 nt small RNA encoded by bacteriophage phi29. We discovered that this small RNA plays a novel and essential role in viral genome DNA packaging (Guo et al., Science 1987; 236:690-694). Six copies of wild type pRNA form a hexameric ring (Guo et al., Mol. Cell. 1998; 2:149-155; Trottier et al., J. Virol. 1997; 71:487-494), which in concert drives the DNA-packaging motor in an ATP-dependent manner (see Guo, Prog in Nucl Acid Res & Mole Biol. 2002; 72:415-472, and Grimes et al., Adv. Virus Res. 2002; 58:255-294 for reviews). Each pRNA molecule contains two functional domains. The intermolecular interacting domain (bases #23-97 at the central region) contains a right hand loop and a left hand loop (Reid et al., J Biol Chem 1994; 269:5157-5162; Chen et al., J Biol Chem 2000; 275(23):17510-17516; Garver et al., RNA. 1997; 3:1068-1079; Chen et al., RNA 1999; 5:805-818). The sequence specific interaction between these two interlocking loops is essential for the pRNA multimer formation. In addition, the sequences of these two loops can be manipulated at will in order to form stable dimer, trimer or hexamer in the presence of Mg²⁺. The second domain is a double-stranded helical structure located at the 5′/3′ paired ends, which is essential for the function of pRNA in DNA packaging by the phi29 motor (Zhang et al., Virology 1994; 201:77-85). These two domains fold independently of each other. We found that removal of the DNA-packaging domain does not alter the properties of pRNA's intermolecular interactions. For example, replacement or insertion of nucleotides preceding nucleotide #23 or following nucleotide #97 does not interfere with dimer, trimer, and hexamer formation (Hoeprich et al., Gene Therapy 2003; 10(15):1258-1267; Chen et al., RNA, 1999; 5:805-818; Shu et al., J Nanosci and Nanotech (JNN) 2003; 3:295-302). Therefore, the 5′/3′ double-stranded helical domain of pRNA can be utilized to carry foreign sequences (Zhang et al., RNA 1995; 1:1041-1050).

Three dimensional computer models of the pRNA monomer, dimer, trimer and hexamer have been constructed in our lab (Hoeprich et al., J Biol. Chem. 2002; 277(23):20794-20803). The construction of these 3D model was based on a variety of experimental data, including chemical modification (Zhang et al., Virology 2001; 281:281-293; Trottier et al., RNA 2000; 6:1257-1266), photoaffinity crosslinking (Garver et al., RNA 1997; 3:1068-1079), complementary modification (Zhang et al., Virology 1994; 201:77-85; Zhang et al., RNA 1995; 1:1041-1050; Zhang et al., RNA 1997; 3:315-322; Wichitwechkarn et al., J Mol. Biol 1992; 223:991-998; Reid et al. J Biol Chem 1994; 269:18656-18661) chemical modification interference (Mat-Arip et al., J Biol Chem 2001; 276:32575-32584), nuclease probing (Reid et al., J Biol Chem 1994; 269:5157-5162; Chen et al., J. Virol. 1997; 71:495-500), competition assays (Trottier et al., J. Virol. 1997; 71:487-494), oligo targeting (Zhang et al., Virology 1995; 211:568-576), and cryo-atomic force microscopy (Chen et al., J Biol Chem 2000; 275(23):17510-17516; Trottier et al., RNA 2000; 6:1257-1266)(Mat-Arip et al., J Biol Chem 2001; 276:32575-32584). Our understanding of the unique structural features of phi29 pRNA allows for to further manipulation of the pRNA molecule, converting it into a vehicle to carry additional RNA sequences that also could serve as scaffold of gene targeting RNA complexes.

We found recently that fusing pRNA with receptor-binding RNA aptamer, folate (Guo et al. Gene Ther. May 2006; 13(10):814-20) [Example 13]), small interfering RNA (siRNA) (Guo et al., Human Gene Therapy 2005; 16:1097-1109 [Example 12])(Khaled et al., Nano Letters 2005; 5:1797-1808 [Example 11]) and ribozyme (Hoeprich et al., Gene Therapy 2003; 10(15):1258-1267) did not disturb the dimer formation or interfere with the function of the inserted moieties. pRNA ribozyme chimera targeting HBV poly A signal exhibited enhanced inhibitory effects of HBV replication, compared with regular ribozyme (Hoeprich et al., Gene Therapy 2003; 10(15):1258-1267). In addition to the superiority of the chimeric pRNA subunit, we also reported that pRNA can be used as a building block for bottom-up assembly in nanotechnology (Shu et al., Nano Letters 2004; 4: 1717-1724). The incubation of trivalent nanoscale particles containing the receptor-binding motif resulted in the binding and co-entry of the therapeutic particles into cells, subsequently modulating the apoptosis of the targeted cells. The usage of such protein-free nanoparticles assembled from pRNA would greatly reduce the antigenicity, allowing for repeated long-term administration. The size of pRNA based nanostructures would avoid the problems of short retention time of small molecules and the difficulties in delivering particles larger than 100 nm. In this report, we extend the choice of therapeutic RNA from siRNA to hammerhead ribozyme targeting survivin, which shows specific down-regulation of the targeted gene and induces programmed cell death without causing non-specific toxicity.

### Materials and Methods

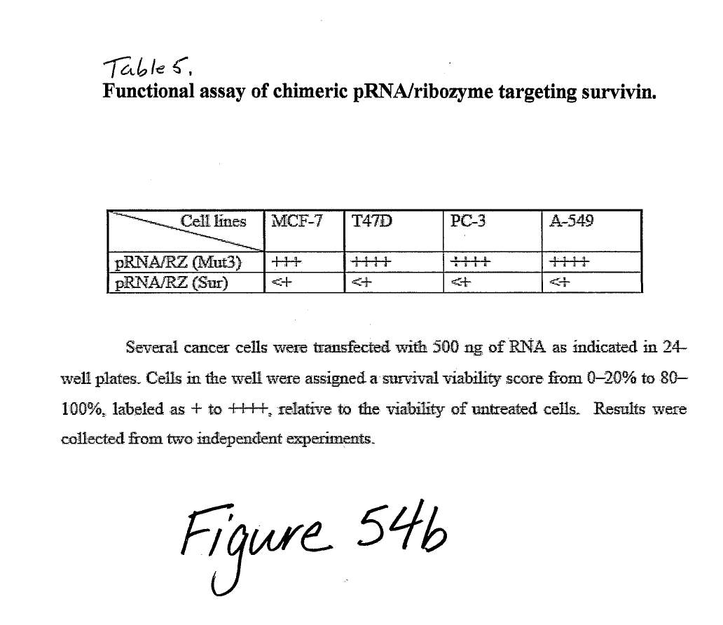

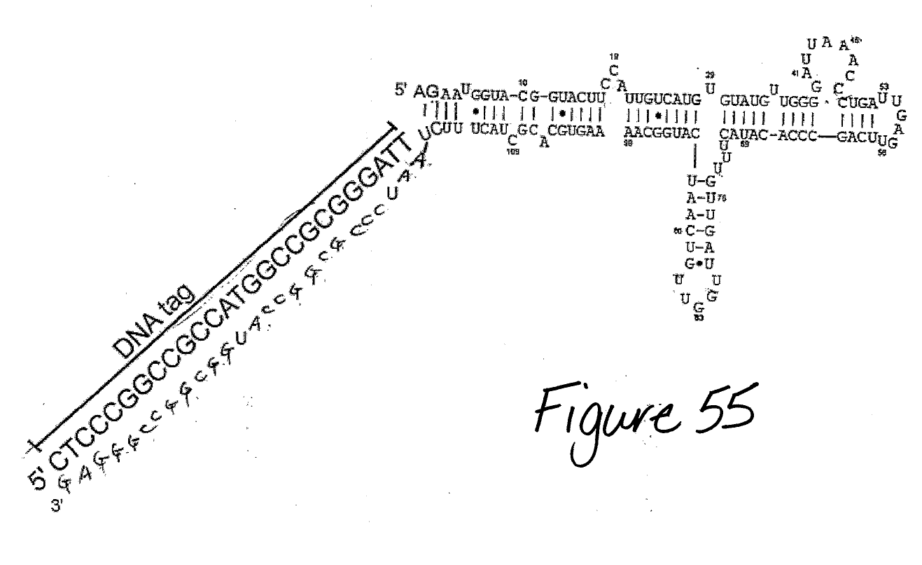

In vitro synthesis of RNA. The synthesis of RNA was described previously (Zhang et al., Virology 1994; 201:77-85). In brief, DNA oligos (Table 4; FIG. 54A) purchased from IDT were used to produce a dsDNA template by PCR. The DNA products containing the T₇ promoter were used as a template for direct in vitro RNA transcription. All RNA was gel purified and resuspended in DEPC treated H₂O. 10 mM of magnesium was included in all buffers to maintain the intermolecular interaction and folding of pRNA (Chen et al., J Biol Chem 2000; 275(23):17510-17516; Mat-Arip et al., J Biol Chem 2001; 276:32575-32584). The nomenclature of pRNA and the resulting chimeric pRNA subunits for the construction of deliverable RNA nanoparticles have been reported (Chen et al., RNA 1999; 5:805-818; Khaled et al., Nano Letters 2005; 5:1797-1808 [Example 11]). pRNA/RZ(Sur) represents a pRNA chimera that harbors a hammerhead ribozyme targeting survivin, following the same strategy for the construction of pRNA/ribozyme (HBV), a chimeric RNA with a pRNA-based vector to carry a hammerhead ribozyme for successful cleavage of the hepatitis B virus (HBV) polyA signal (Hoeprich et al. Gene Therapy 2003; 10(15):1258-1267). pRNA/ribozyme(HBV)(Hoeprich et al., Gene Therapy 2003; 10(15):1258-1267) is also used in this study, and referred as pRNA/RZ(mut3). Assay for gene silencing efficiency of pRNA/RZ (Sur) by transfection.

The methods for the transfection of cells with pRNA chimera have been reported (Khaled et al., Nano Letters 2005; 5:1797-1808 [Example 11]). Human cervical cancer cells Hela T4 were plated in a 24-well culture dish and incubated overnight at 37° C./5% CO₂. The following morning, the medium was replaced with an antibiotic-free medium and the cells were transfected separately with pRNA constructs at 0.5 μg, 0.1 μg or 0.02 μg using Lipofectamine 2000 (Invitrogen) with three duplicates per treatment. After six hours, the transfection solution was replaced with a standard medium supplemented with 10% FBS and antibiotics. Tetrazolium-based MTT assays were performed to determine the cell viability. Similar assays were performed for the KB, LNCaP and MDA-MB-231 cells.

Western Blot assay. T47D human breast cancer cells were seeded in 60 mm dishes and grown to 70% confluency in DMEM supplemented with 10% FBS and penicillin/streptomycin. Prior to transfection, cells were switched to antibiotic free medium and then transfected with pRNA chimera targeting survivin, or mutant chimeric ribozyme, as negative control. Lipofectamine 2000 was used according to the manufacturer's instructions. Cells were rinsed and harvested in lysis buffer at 12, 16, 20, and 24 hours after transfection. Protein concentrations were determined and equal amounts of protein were loaded into a 12% polyacrylamide gel. Proteins were resolved and transferred to a nitrocellulose membrane using semi-dry transfer (BioRad). Membranes were blocked, incubated with primary antibody peroxidase, and conjugated to a secondary antibody according to the manufacturer's instructions (Amersham ECL kit). Following activation of the chemiluminescent probe, membranes were exposed to film.

Assay for apoptosis of MCF-7 induced by pRNA/RZ(Sur) using PI/annexin V double staining in flow cytometry. The methods for apoptosis assay have been reported (Guo et al., Human Gene Therapy 2005; 16:097-1109 [Example 12]). Briefly, human breast cancer cells MCF-7 were grown in DMEM medium supplemented with 10% FBS and penicillin/streptomycin, and plated into 24-well plates at a density of 0.5×10⁵ cells per well. Transfections were performed with a 0.5 μg ribozyme per well and three duplicates per treatment. 48 hours after transfection, apoptosis in breast cancer cell MCF-7 was assessed with the annexin V-propidium iodide (PI) double staining method.

Real-time PCR assay. MCF-7 cells were seeded into 24-well plates at a density of 0.5×10⁵ cells per well. Transfections were performed with a 0.5 μg RNA per well and three duplicates per treatment. Cells were harvested 48 hours after transfection and total RNA was extracted with a QIAamp RNA kit (Qiagen). Reverse transcription was carried out on 1 μg of RNA with RevertAid™ First Strand Synthesis Kit (Fermentas).

Equal amounts of cDNA were submitted to PCR, in the presence of SYBR green dye with the QuantiTect SYBR Green RT-PCR Kit (QIAGEN) and the ABI PRISM 6700 Real time PCR detection machine (Fengling Biotechnology Inc.). Primers for survivin were 5′-AAA GAG CCA AGA ACA AAA TTG C-3′ and 5′-GAG AGAGAA GCA GCC ACT GTT AC-3′, which were published previously (Weikert et al., Fertil. Steril. 2005 April 1983; 1100-1105). PCR was performed by 40 cycles of 0.5 seconds at 95° C., 10 seconds at 60° C. and 10 seconds at 72° C. PCR without template was used as a negative control. The β-actin endogenous housekeeping gene was used as an internal control. Both β-actin and negative control were amplified on the same plate as the experimental gene of interest. Each sample was normalized by using the difference in critical thresholds (CT) between survivin and B-actin. The following equation was used to describe the result: ΔΔCTₛᵤᵣᵥᵢᵥᵢₙ=ΔCTₛᵤᵣᵥᵢᵥᵢₙ-ΔCT_{β-actin} where ΔCTₛᵤᵣᵥᵢᵥᵢₙ was the difference in CT between survivin and negative control, and ΔCT_{β-actin} was the difference between β-actin and negative control. The mRNA levels of each sample were then compared using the expression 2ΔΔCTₛᵤᵣᵥᵢᵥᵢₙ. The results of each group were averaged. The expression level for non-transfected sample was arbitrarily assigned value 1 and the final results were expressed as fold number compared to non-transfected sample.

In vitro cleavage by hammerhead ribozyme. The survivin mRNA targeting ribozyme pRNA/RZ (Sur) and control ribozyme pRNA/RZ (Mut2) cleavage reactions were performed at 37° C. for 60 min in the presence of 20 mM Tris pH 7.5, 20 mM MgCl₂, and 150 mM NaCl. Ribozyme RNA (5 ug) was used to cleave partial sequence of survivin mRNA (200 ng). The 127 nt [³²P] label RNA substrate is expected to be cleaved into a 77 nt and a 50 nt fragment. The cleavage products were separated by 8% PAGE/8 M urea-denaturing gel.

### Results

1. Construction of pRNA Chimera Harboring Ribozyme

To evaluate the effectiveness of therapeutic RNA molecules in treating cancer, it is necessary to suppress genes involved in tumor development and progression. Survivin was chosen as a target since it inhibits apoptosis and is detected only in cancer cells and not in normal adult cells. It has previously been reported that the suppression of survivin induces the apoptosis of cancer cells (Grossman et al., Proc Natl Acad Sci 2001; 98:635-640; Frey et al., J Struct Biol 1999; 127:94-100).

A pRNA/RZ(Sur) RNA chimera based on pRNA vector sequences was constructed to target the survivin mRNA (FIG. 45). A survivin targeting ribozyme was connected to the 5′/3′ ends of pRNA and the pRNA was reorganized as the circularly permuted form (Pennati et al., J Clin. Invest 2002; 109:285-286). As mentioned previously, phi29 pRNA contains an intermolecular interacting domain and a double-stranded helical domain. These two domains fold independently of each other and the addition or deletion of nucleotides at the 5′ end preceding base #23 and at the 3′ end following base #97 does not affect the correct folding and overall structure of pRNA (Zhang et al., RNA 1995; 1:1041-1050). Circularly permuted pRNAs were constructed without affecting its folding (Zhang et al., Virology 1995; 207:442-451). Two linker sequences were used to link the ribozyme with the pRNA vector. The approach of circular permutation is to ensure the independent and collect folding of both the ribozyme and the pRNA vector, and to relocate the nascent 5′/3′ end of the RNA chimera into a tightly folded region, and protect the pRNA from exonuclease digestion. Three mutative chimeric ribozymes were used in this study (FIG. 45).

2. pRNA/RZ(Sur) Chimera Induced Apoptosis and Cell Death Specifically in all Tested Cancer Cells

a. The Effects of Chimeric pRNA/RZ (Sur) in Human Breast Cancer Cells

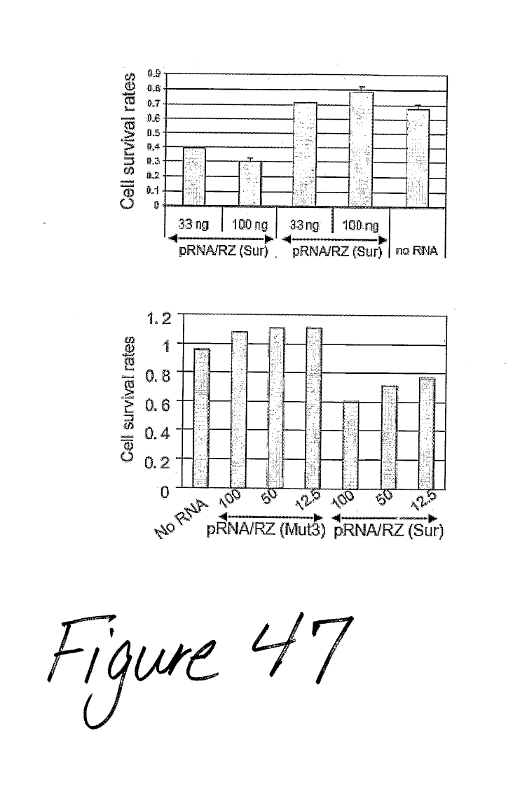

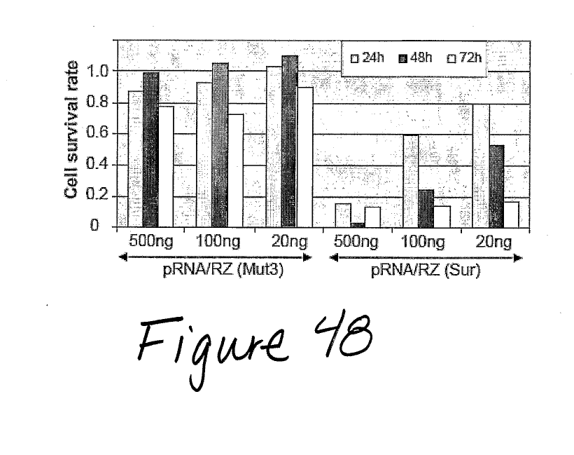

The pRNA/RZ(Sur) was tested on four breast cancer cell lines, MCF-7, T47D, MDA-MD-231 and MDA-MB-453. After transfection, the majority of cells shrank and were detached from the cell culture plate, while the control pRNA/RZ (mut3) did not cause the change of the cells (FIG. 46, and Table 5, FIG. 54B). When the effects of various chimeric ribozymes were measured by an MTT assay in MDA-MD-231 and MDA-MB-453 cells, pRNA/RZ (Sur) showed strong, dose-dependent inhibition of cell viability, while pRNA/RZ (mut3) showed no effects (FIG. 47).

The result indicates that the chimeric pRNAs did not exhibit non-specific cytotoxicity. However, when introduced into breast cancer cells by transient transfection, pRNA/RZ(Sur) could induce cell death specifically in a dose-dependent manner.

b. The Effects of Chimeric pRNA/RZ (Sur) in Human Cervix Cancer Cells

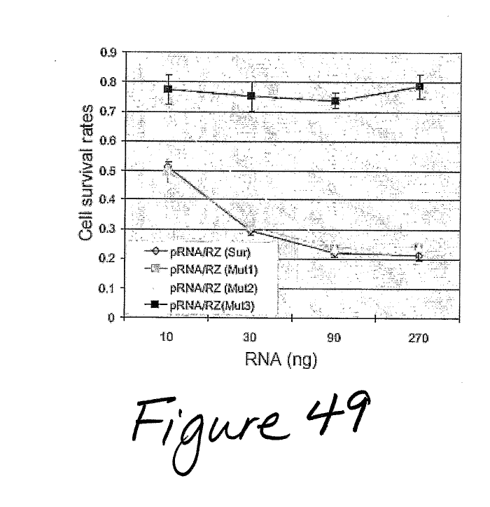

pRNA/RZ(Sur) was introduced into the human cervical cancer Hela T4 cells by transfection in an effort to evaluate its function in inducing apoptosis of human cervix cancer cells. Cell viability was measured at various time points after transfection. As shown in FIG. 48, treatment with pRNA/RZ(Sur) resulted in a dose-dependent decreasing of cell viability compared to no RNA control. For pRNA/RZ(mut3) treated cells, no significant difference in survival rates were observed when different RNA doses were used, indicating that pRNA/RZ(Sur) acts on Hela T4 cells without causing non-specific toxicity.

c. The Effects of Chimeric pRNA/RZ (Sur) in Nasopharyngeal Cancer Cells

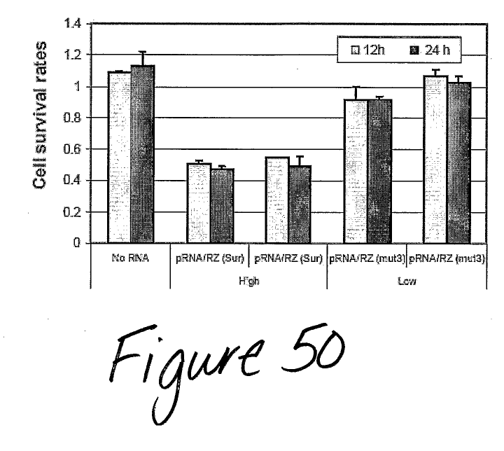

Human nasopharyngeal cancer KB cells were exposed to pRNA/RZ(Sur), as well as pRNA/RZ(mut3) as negative control. As shown in FIG. 49, KB cells responded significantly to pRNA/RZ(Sur) after transfection. The nonspecific toxicity induced by the mutant ribozyme was not significant, even as the dose increased. This indicates that the cell death caused by chimeric survivin ribozyme is specific, as found in other cancer cells.

d. The Effects of Chimeric pRNA/RZ(Sur) in Prostate Cancer Cells

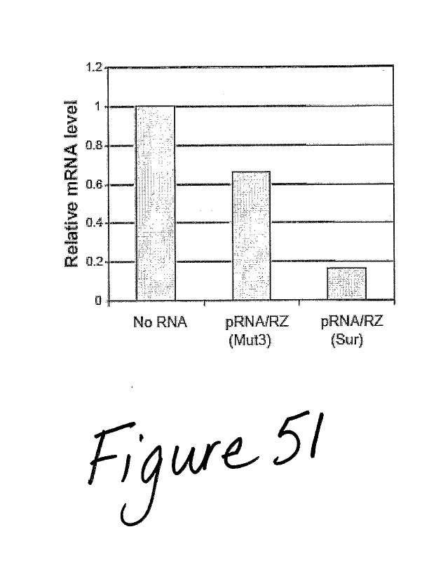

Human prostate cancer cell lines LNCaP were transfected with different doses of pRNA/RZ (Sur) or pRNA/RZ(mut3), with the latter serving as negative control. Twelve hours or 24 hours after transfection, prostate cancer cells reacted strongly only to the treatment of pRNA/RZ(Sur), while the control RNA did not affect cell survival rate significantly (FIG. 50). It suggests that the reduction of cell viability depended on the sequence corresponding to the survivin ribozyme, instead of being caused by the non-specific RNA toxicity.

e. The Effects of Chimeric pRNA/RZ (Sur) on Human Lung Cancer Cells

Human lung cancer line A-549 were transfected with different doses of pRNA/RZ(Sur) or pRNA/RZ(mut3). After transfection, A-549 cells reacted strongly to the treatment of pRNA/RZ(Sur) and fell off the well of the culture plate on second, while the control pRNA/RZ(mut3) did not affect cell survival rate significantly (Table 2). It suggests that the reduction of cell viability depended on the sequence corresponding to the survivin ribozyme, instead of being caused by the non-specific RNA toxicity.

3. Specific Inhibition of Survivin Expression by pRNA/RZ(Sur) in mRNA and Protein Level

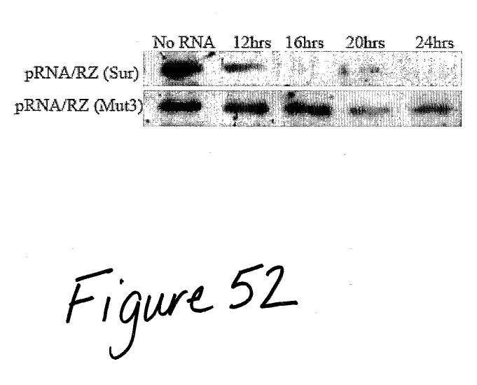

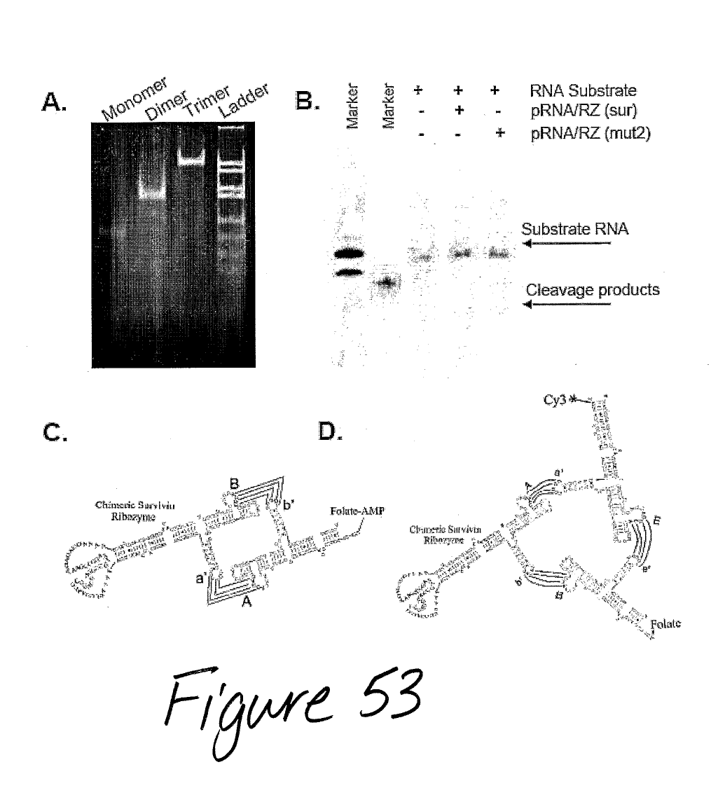

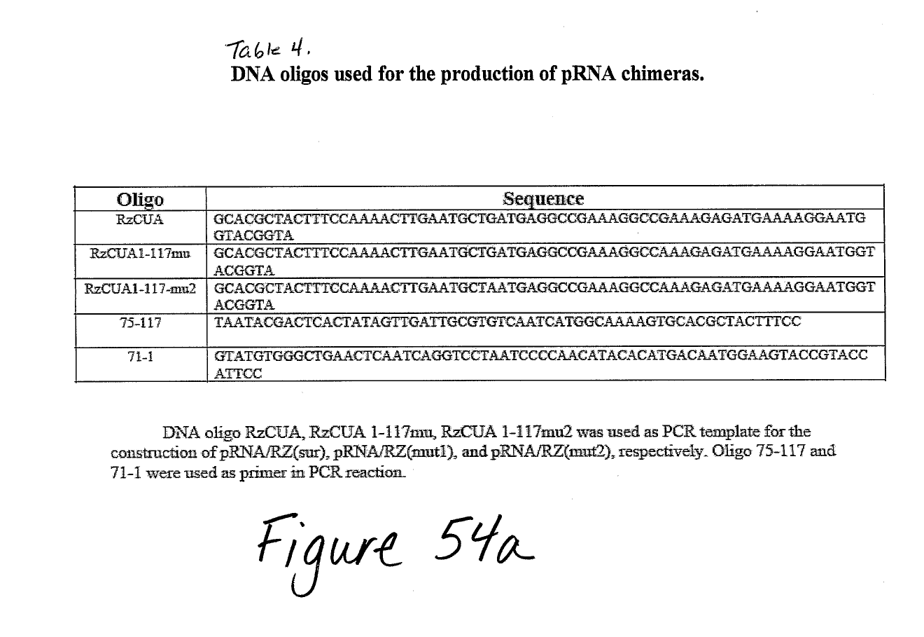

To test its predicted function in suppressing the expression of survivin, pRNA/RZ(Sur) was introduced into MCF-7 and T47D human breast cancer cells in which survivin was abundantly expressed. Both Real-time PCR and immuno-blotting analysis revealed that the mRNA and protein expression of survivin were significantly reduced and almost totally eliminated 16 hours after transient transfection (FIGS. 51 and 52). In contrast, neither nonspecific mutant control treated cells nor untreated cells were shown to significantly decrease survivin expression, further demonstrating the specificity with which the pRNA/RZ(Sur) acted. As shown in FIG. 52, treatment with pRNA/RZ(Sur) resulted in a time-dependent reduction of the survivin protein compared to the control groups. The specific cleavage of survivin mRNA by pRNA/RZ(Sur) was shown in FIG. 53B. The specificity was demonstrated since the mutant pRNA/RZ (mut2), which contains a two-base mutation in the catalytic core, did not produce RNA cleavage product.

### 4. The Chimera Cause Apoptosis Instead of Necrosis

To determine whether pRNA/RZ(Sur) induces apoptosis caused by the silence of the anti-apoptosis factor survivin, or promotes the necrosis nonspecifically, annexin V-propidium iodide (PI) double-staining was performed, followed by flow cytometry analysis on breast cancer cells transfected with pRNA/RZ(Sur). As shown in FIG. 46B, 25%±8.6 of MCF-7 cells underwent apoptosis after RNA treatment, as shown in the cell population in the lower right quadrant representing apoptotic cells. On the contrary, cells treated with mutant 3 show only a slight increase (3.6%±0.2) of apoptotic cells, compared to (2.1%±0.3 ) of cells treated with pRNA vector alone. The necrotic cells, as indicated in the upper right quadrant, did not show marked increase after RNA treatment, which indicates the RNA/ribozyme caused apoptosis instead of necrosis in cancerous cells in the early stage of RNA treatment.

5. Testing of the Safety of the pRNA Chimera.

The safety of the pRNA chimera was tested by using a high dose of pRNA chimeric in both the incubation and transfection experiment. Incubation of cells with varied concentrations of pRNA chimera did not cause noticeable toxicity to cells (see FIG. 37; Example 12). Incubation of cancer cells with pRNA/siRNA (survivin) or pRNA/RZ(Sur) did not cause cell death for up to 72 hours in the absence of transfection reagent (Guo et al. Human Gene Therapy 2005; 16:097-1109 [Example 12]. This indicates that the chimeric pRNAs did not show nonspecific cytotoxicity on incubation. However, when introduced into cells by transfection, the pRNA chimera caused the death in cells derived from breast cancer, prostate cancer and lung cancer. But only chimeric pRNA containing survivin ribozyme sequence caused significant inhibition of cell viability. As shown in FIG. 48, 49, 50 and 51, the control pRNA/RZ(mut3), which inhibits the replication of hepatitis B virus and contains vector sequences identical to pRNA/RZ(Sur) except the ribozyme sequence, did not show marked inhibitory effect to cell growth even in high RNA concentration.

6. Competence of pRNA/RZ(Sur) in the Assembly of Dimer and Trimers Nanoparticles

Specific cell recognition and specific gene silencing are required for effective RNA-based targeted therapy. However, fusion or conjugation of functional RNA molecules may lead to misfolding and loss of function. pRNA dimer construction is an alternative approach for achieving these two aspects. Ideally, the additional RNA motif or chemicals can be incorporated into the delivery complex to carry out other tasks such as endosome disruption, detection of the cell fate following the treatment, and enhancement of therapeutic effect by multiple targeting. Recently, multivalent RNA complex has been constructed using phi29 pRNA chimera. Dimer or trimer was assembled by interlocking loop/loop interaction of the engineered chimeric pRNA harboring receptor-binding RNA aptamer or siRNA (Guo et al. Human Gene Therapy 2005; 16:097-1109 [Example 12]; Khaled et al., Nano Letters 2005; 5:1797-1808 Example 11]). The major goal of constructing pRNA/RZ(Sur) is to design one subunit building block for dimer or trimers of pRNA chimeras as delivery nanoparticles. pRNA/RZ(Sur) was found to be competent in dimer and trimer formation, as documented by native gel electrophoresis (FIG. 53) and other physical approaches (data not shown) such as ultracentrifugation and single molecule counting. Formation of dimer was achieved by mixing pRNA/RZ(Sur)(A-b′) with pRNA(B-a′). Formation of trimer was achieved by mixing pRNA/RZ(Sur)(A-b′) with pRNA(B-e′) and (E-a′) (Guo et al., Mol. Cell. 1998; 2:149-155; Chen et al., RNA 1999; 5:805-818; Zhang et al., Mol Cell 1998; 2:141-147). It shows that stable RNA dimer or trimer is generated from chimeric RNA monomeric building blocks, despite the addition of survivin ribozyme to pRNA vector sequence. Therefore, pRNA/RZ(Sur) can be used to assemble the dimeric/trimeric RNA nanoparticles and will be an additional member of the polyvalent RNA delivery system.

### Discussion

RNA therapeutics has been thought to be one of the most promising approaches in modern medicine. As in other therapeutics, toxicity and specificity are two major issues in the development. We have put our effort into the quest for low toxicity therapeutic RNA complex. Previously, we found that phi29 pRNA can be a vector to escort the ribozyme for inhibition of hepatitis virus B replication (Hoeprich et al., Gene Therapy 2003; 10(15):1258-1267). The pRNA/RZ(Sur) was found to be efficient in inducing specific cell death. Since each therapeutic RNA contains a unique sequence, the safety issue depends on the type of cells, and is a case-by-case issue. For example, breast cancer cell lines MCF-7 are far more fragile and more sensitive to RNA transfection, compared to other breast cancer cell lines. Therefore, we have tested a variety of cancer cell lines, including breast cancer, prostate cancer, cervix cancer, nasopharyngeal cancer, and lung cancer. The pRNA/RZ(Sur) was found to be very efficient at inducing cancer cell apoptosis. However, we cannot say this RNA chimera is safe for all cell types. We have also tested this RNA chimera on the most fragile and sensitive MCF7 cell by real time PCR to evaluate the silence efficiency on the transcript of survivin gene. We found that even though the molecule can silence the expression of survivin gene specifically, the expression of survivin mRNA is also slightly reduced by a mutant (FIG. 51). Obviously, such reduction is caused by nonspecific RNA effect. Thus, the safety issue must be addressed again. Since molecular therapy involves the delivery of therapeutic agents into the cell, specificity is more critical than the toxicity, since the goal in cancer therapy is to eliminate cancer cells. The nonspecific inhibition of cancer cells might, in some cases, be desirable as long as the pRNA can enter the cancer cell specifically and as long as the un-entered pRNA is not toxic to the cells. From our previous report, we found that incubation of cells with high concentration of pRNA chimera did not cause noticeable cell death. Thus, pRNA chimera is promising in that it can enter the cell specifically by being engineered into multimer. Our effort will focus on the specificity of cell entry. The advantage of using phi29 pRNA chimera is to develop a powerful method of specific delivery of RNA chimera to target cells; thus, a balance between the effect in cell killing and the efficiency of cell entry will be assessed.

As we know, phi29 pRNA has a tendency to form dimers (a linking of 2 pRNA), trimers (3 pRNA), and hexamers (6 pRNA) as a result of the interaction of interlocking loops. Thus, two to six pRNA chimeras can be incorporated into the RNA nanocomplex, with multiple positions available to carry RNA molecules for targeting, therapy, or detection. For example, one subunit of the complex could be altered to carry an RNA aptamer that binds the cell surface receptor, or a ligand such as folate (Guo et al. Gene Ther. 2006. May; 13(10):814-20) Example 13), thereby helping to carry the RNA complex for cellular entry. The remaining subunits could be modified to carry specific therapeutic siRNAs, ribozymes, antisense RNAs, chemotherapy drugs, fluorescent dyes, heavy metals, quantum dots, or radioisotopes for cancer cell elimination or detection. Endosome-disrupting chemicals may also be incorporated into the RNA complex to promote the release of RNA from the endosome. In addition, the use of these RNA nanoparticles (with sizes of 30-40 nanometers) avoids the problem of a short half-life encountered in vivo by smaller molecules due to short retention times and also avoids the problem of poor delivery efficiency encountered by larger molecules (greater than 100 nanometers). It has been well accepted that immunogenicity of RNA is in a very low level, except when complexed with protein (Goldsby et al. Immunology, pp. 57-61 (W. H. Freeman and Company, New York, 2002). The construction of protein/peptide-free nanoparticles can avoid the immune response, which could allow long-term administration.

### Example 15

### Incorporation of Nucleotide Derivatives into Chimeric pRNA to Increase Stability

Nucleotide derivatives such as 2′-NH₂—, 2′-CH₃—, or 2′-F-2′ deoxy CTP; 2′-F-2′ deoxy UTP or spiegelmer (L-nucleotide aptamers) can be incorporated into chimeric pRNA to produce stable transcripts that are resistant to RNase digestion in serum. The stabilizing modification can be made at the 2′ position or at other positions. Stabilization of pRNA is advantageous for in vivo therapeutic applications, where it is important that the pRNA nanoparticles are stable under a variety of biological conditions and are resistant to digestion by RNases in serum. Native phage phi29 pRNA is susceptible to degradation by various nucleases upon contact with biological samples. pRNA analogs can be constructed with modified nucleotides that are expected to give rise to enhanced bio-stability. More generally, functional pRNA with 2′-F-NTP, 2′-NH₂-NTP or 2′-CH₃-NTP represent examples of modified pRNAs that can be constructed.

From a dsDNA template containing a T7 promoter and the 117-nt minimal wild-type pRNA sequence, pRNA analogs can be prepared using, for example, 2′-F-NTP or 2′-NH₂-NTP (Trilink Biotechnologies). In particular, nucleotide derivatives such as 2′-NH₂-2′-CH₃— or 2′-F-2′ deoxy CTP; 2′-F-2′ deoxy UTP; or spiegelmer can be incorporated into RNA to produce stable in vitro RNA transcripts that are resistant to RNase digestion. An example of such a stabilized pRNA is one that contains one or two types of unmodified (naturally occurring) nucleotides and one type of 2′-F—; 2′-NH₂—; or 2′-CH₃— spiegelmer nucleotides. Stabilized chimeric pRNAs are expected to enter cancer cells when they are present as a component of a pRNA dimer, trimer or hexamer that carries a cell receptor binding ligand.

Stabilized pRNA chimera that exhibit favorable receptor binding affinities can be further analyzed by in vitro functional tests—such as the ability for dimer, trimer or hexamer formation, the specificity for entering cell, and the activity of specific gene silencing. Chimeric pRNA can be tested for effectiveness in inhibiting survivin or Bcl-2 expression in a number of breast cancer cell lines.

Synthetic 2′-NH₂, 2′-CH₃, or 2′-F-2′-deoxy CTP, 2′-F-2′-deoxy UTP or spiegelmer modified RNA pieces can also be used to re-constitute functional pRNA. 2′-F-2′-deoxy nucleotides are available from Epicentre.

### Example 16

### pRNA Chimera with Reduced Toxicity

Toxicity and specificity are two major concerns that need to be addressed for all therapeutic technology, including technology based on the use of RNA.

### 1. Circular Permutation

We propose that by identifying the optimal circular permutation for a circularly permuted pRNA chimera, specificity can be increased and/or toxicity reduced.

The toxicity of a pRNA may be affected by its primary sequence, the secondary structure, and three-dimensional structure, and the location for the opening of the location of the 5′ and 3′ ends. Using circular permutation, a variety of pRNA with different 5′ and 3′ termini can be produced, but with identical sequence and three-dimensional structure. Thus, chimeric pRNA that carry a biologically active moiety (e.g., ribozyme, antisense, or aptamer) in the spacer region can be fashioned into variety of circularly permuted RNAs. For a chimeric pRNA with 120 nucleotides in the pRNA region, 120 varieties of circularly permuted RNAs with identical sequences, function, and three dimensional structures will be produced. These species can be screened to identify the lower toxicity species that exhibit full gene silencing function and the capability to form dimer or trimer in nanoparticles construction.

2. pRNA with DNA Annealed to 3′ End

Another method for reducing toxicity involves identifying chimeric pRNAs that are less toxic than their counterparts. pRNA chimera sph1-pRNA was formed by attaching a 26 nucleotide single-stranded RNA fragment (5′ AAUCCCGCGGCCAUGGCGGCCGGGAG 3′) to the 3′ end of a pRNA. The pRNA chimera was then contacted with a DNA oligonucleotide that was complementary to the RNA fragment, under conditions to allow annealing of the DNA oligonucleotide to the RNA fragment. The resulting pRNA construct (FIG. 55), which includes the annealed oligonucleotide, was found to have reduced toxicity.

Similarly, sph1-pRNA/siRNA is a chimeric pRNA/siRNA with an extra 26 nucleotide RNA single strand fragment at its 3′ end. The extra RNA fragment was annealed with a 26 nucleotide complementary DNA oligonucleotide. The resulting sph1-pRNA/siRNA (including the 26 nucleotide complementary DNA oligonucleotide) was found to have (1) reduced toxicity and (2) retained gene silencing function. Reduced toxicity was demonstrated by MTT assay, and the gene silencing function was demonstrated using GFP and dual-luciferase assays.

### Stability Studies

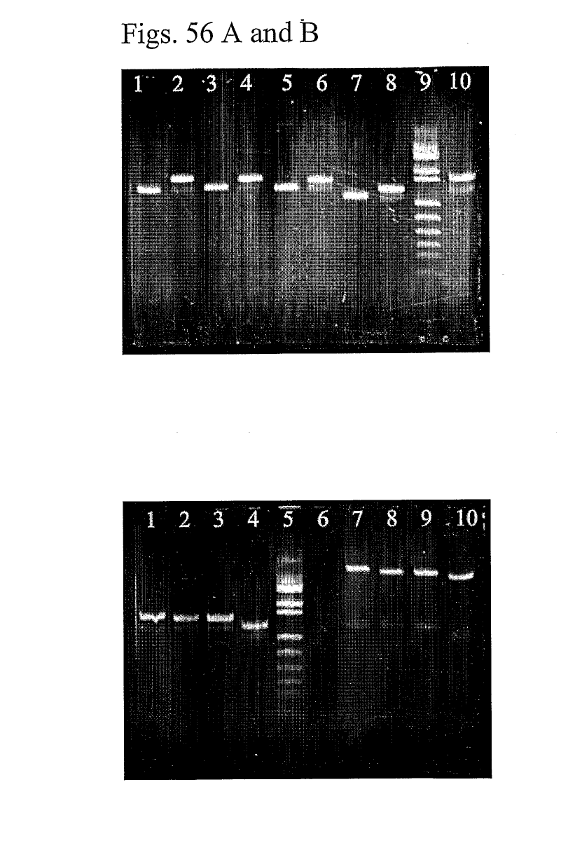

The pRNAs used in the toxicity studies contained the 3′ RNA fragment extension and the annealed complementary DNA oligonucleotide (FIG. 55). These pRNAs were first analyzed for stability and for annealing efficiency, using gel electrophoresis (8%PAGE/Urea).
Legend for gel shown in FIG. 56A
   1. Sph1-pRNA
   2. Sph1-pRNA annealed with 117-143 oligonucleotide
   3. Sph1-pRNA/siRNA(luciferase)
   4. Sph1-pRNA/siRNA(luciferase) annealed with 117-143 oligonucleotide
   5. Sph1-pRNA/siRNA(GFP)
   6. Sph1-pRNA/siRNA(GFP) annealed with 117-143 oligonucleotide
   7. Sph1-pRNA/siRNA(survivin)
   8. Sph1-pRNA/siRNA(survivin) annealed with 117-143 oligonucleotide
   9. Ladder
   10. Sph1-pRNA annealed with 117-143 biotinylated oligonucleotide
      No significant degradation of RNA was found, as revealed by FIG. 56A. It can also serve as a loading control of the RNA toxicity assay.
Legend for gel shown in FIG. 56B
   1. Sph1-pRNA annealed with 117-143 biotinylated oligonucleotide
   2. Sph1-pRNA/siRNA(luciferase) annealed with 117-143 biotinylated oligonucleotide
   3. Sph1-pRNA/siRNA(GFP) annealed with 117-143 biotinylated oligonucleotide
   4. Sph1-pRNA/siRNA(survivin) annealed with 117-143 biotinylated oligonucleotide
   5. Ladder
   6. (empty)
   7. #1+streptavidin
   8. #2+streptavidin
   9. #3+streptavidin
   10. #4+streptavidin

The annealing efficiency was determined by incubating four biotin RNAs with streptavidin and look at the migration rate change. FIG. 56B shows that the majority of the PAGE-purified RNAs carried 3′ end DNA oligonucleotide.

### Toxicity Studies

To determine whether the annealing of a DNA oligonucleotide at 3′ end of a pRNA can lower the RNA toxicity, toxicity studies were performed in cell line PC3, using RNA concentrations of 40 nM, 13.3 nM, 4.44 nM, and 1.48 nM. The MTT assay (available from Promega) was used to determine the cytotoxicity of the various constructs. Briefly, the various pRNA constructs are transfected into the cells. The viability of the cells is measured using different concentrations of pRNA construct. At a given concentration, the RNA construct with highest toxicity will kill most of the cells. RNA constructs with no or low cytotoxicity will have no or low effect on cell viability, and the cell viability reading is similar as the untreated control group.

### Results

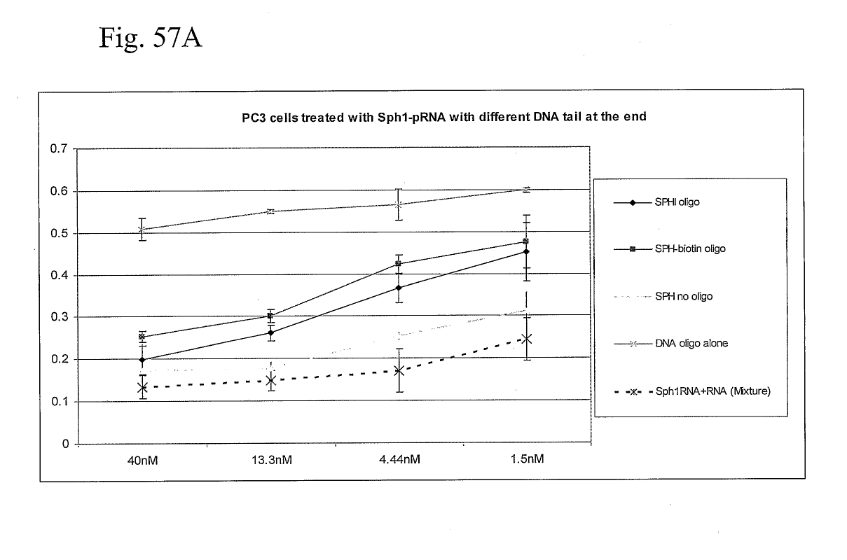

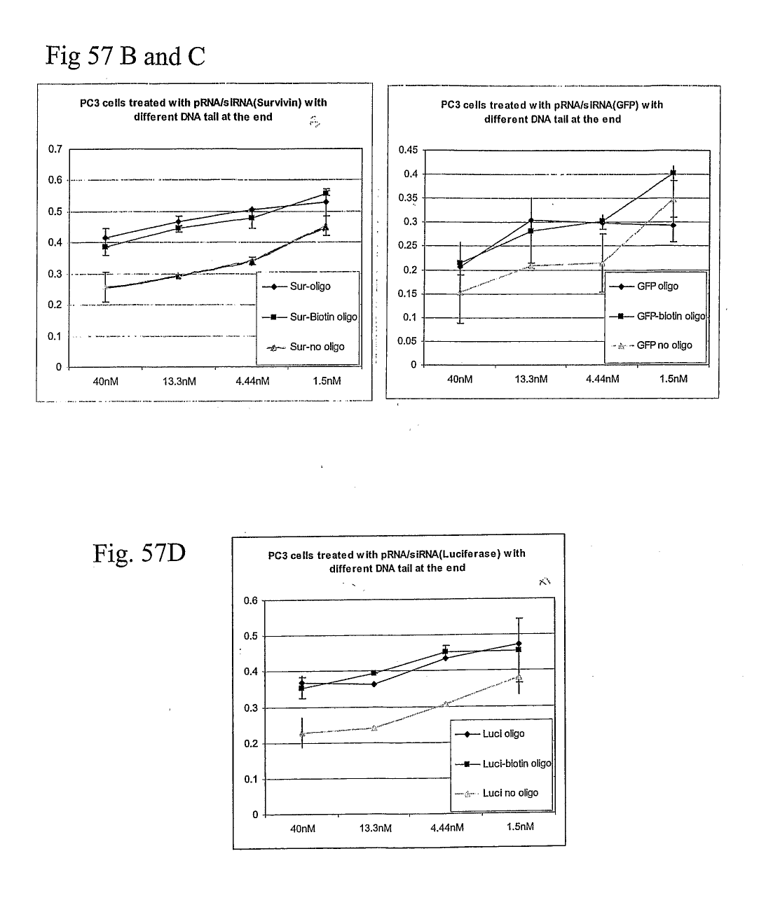

FIG. 57A shows toxicity assay results for Sph1-pRNA (triangle); Sph1-pRNA annealed with a 117-143 DNA oligonucleotide, PAGE purified (diamond); Sph1-pRNA annealed with a 117-143 biotinylated DNA oligonucleotide, PAGE purified (square); 117-143 DNA oligonucleotide alone (X); and Sph1-pRNA mixed with 117-143 DNA oligonucleotide, NOT purified (star/dashed line).

Annealing of the 117-143 DNA or 117-143 biotinylated DNA oligonucleotide was found to reduce the cytotocity of sph1-pRNA. The decreased toxicity was achieved by annealing the DNA oligonucleotide with sph1-RNA, rather than using a mixture of DNA oligonucleotide and sph1-pRNA (non-PAGE purified). The DNA oligonucleotide alone had little effect on cell viability. The integrity of RNA is assured by the PAGE gel, as indicated in above. The results are shown in FIG. 57A.

The other three sets of RNA toxicity experiments (FIGS. 57B, C and D) also indicate that the toxicity of Sph-pRNA/siRNA can be reduced by annealing with a DNA oligonucleotide.

### Gene Silencing

The ability of the sph1-pRNA/siRNA chimera to silence gene expression was evaluated. Sph1-pRNA/siRNA(GFP) and sph1-pRNA/siRNA(lucifierase) were used to test the ability of the dimeric pRNAs to silence green fluorescent protein (GFP) and luciferase (Luci), respectively. pRNA/siRNA(GFP) and pRNA/siRNA(luciferase) were used as controls. It was found that both sph1-pRNA/siRNA(GFP) and sph1-pRNA/siRNA(Luci) pRNA chimeras silenced their respective target genes in a dose dependent manner, regardless of whether the pRNA chimera contained the heterologous oligonucleotide at the 3′ end.

### Material and Methods

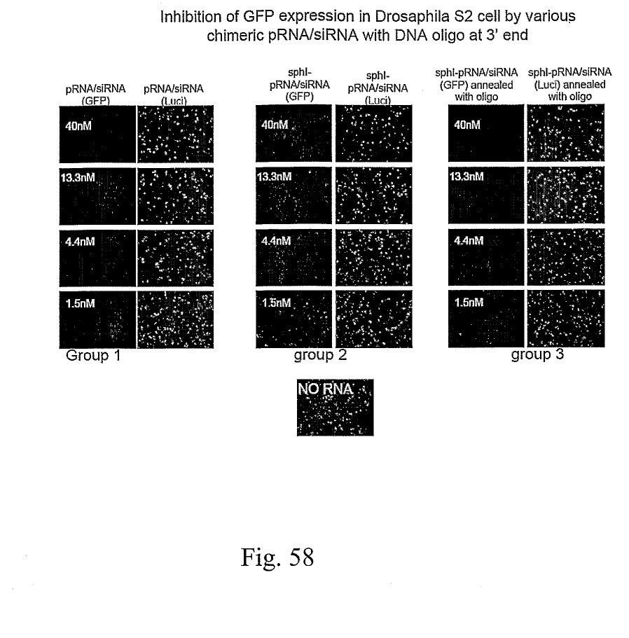

Drosophila S2 cell were seeded in 24 well plate at 0.6×10(6) per well. The next day, plasmid pMT-GFP was co-transfected with the various chimeric pRNA complexes at the concentrations indicated in FIG. 58. GFP expression was induced by GuSO4. Images were taken using fluorescence microscopy.

### Results

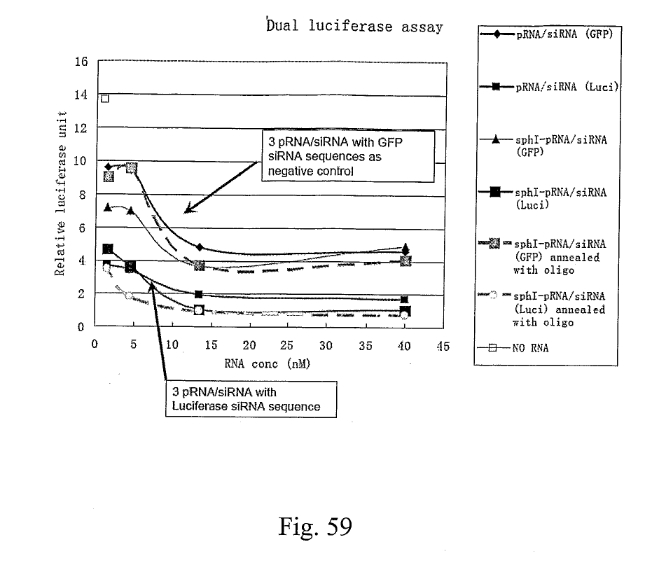

Both sph1-pRNA/siRNA and sph1-pRNA/siRNA annealed with DNA oligo were effective to silence GFP gene expression in a dose depend manner (FIG. 58; left panels of groups 2 and 3, respectively; FIG. 59). The knockdown effects were sequence specific, since all three RNA containing luciferase siRNA sequences did not show any inhibitory effect on GFP expression (FIG. 58; right panels).

### Sequence Listing Free Text

1 organism name: Bacteriophage phi29/PZA
2 circularly permuted pRNA from bacteriophage phi29 (short loop)
3 RNA chimera containing phi29 pRNA and hammerhead ribozyme
4 U7snRNA substrate
5 anti-12-Lox ribozyme
6 Lox substrate RNA
7 pRNA chimera
8 linking loop
9 U7 substrate
10 RzU7 hammerhead ribozyme
11 organism name: Bacteriophage SF5′
12 organism name: Bacteriophage B103
13 circularly permuted pRNA from bacteriophage phi29 (long loop)
14 organism name: Bacteriophage M2/NF
15 organism name: Bacteriophage GA1
16 aptRNA
17 RNA chimera containing phi29 pRNA and hammerhead ribozyme
18 RzA hammerhead ribozyme
19-22 3′ pRNA extension
23 hammerhead ribozyme
24 Hepatitis B virus polyA substrate
25 RNA chimera containing phi29 pRNA and hammerhead ribozyme
26 Wild-type pRNA with base pair change at base of stem structure.
27 Wild-type pRNA
28 Synthetic permuted SF5 pRNA CHIMERA
29 Synthetic permuted B103 pRNA CHIMERA
30 Synthetic permuted SF5 pRNA CHIMERA
31 Synthetic permuted M2/NF pRNA CHIMERA
32 Synthetic permuted GA1 pRNA CHIMERA
33 Synthetic permuted aptamer pRNA CHIMERA
34 Synthetic circularly permuted pRNA
35 Synthetic circularly permuted pRNA
36 Synthetic circularly permuted pRNA
37 Synthetic cpRNA transcript
38 Synthetic DNA template
39 Synthetic cpRNA transcript
40 Synthetic phi29 viral particle
41 Synthetic chimeric pRNA
42 Synthetic chimeric pRNA
43 Synthetic chimeric pRNA
44 Synthetic chimeric pRNA
45 Synthetic pRNA chimera

The complete disclosures of all patents, patent applications including provisional patent applications, and publications, and electronically available material (e.g., GenBank amino acid and nucleotide sequence submissions) cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

1. A pRNA chimera comprising a paired double-stranded helical domain and an intermolecular interaction domain, wherein the paired double-stranded helical domain comprises a heterologous component comprising an siRNA.

2. The pRNA chimera of claim 1 wherein the siRNA is effective to silence a gene expressed in a cancer cell.

3. The pRNA chimera of claim 2 wherein the gene encodes survivin.

4. The pRNA chimera of claim 1 wherein the siRNA is effective to silence a viral gene.

5. A pRNA chimera comprising a paired double-stranded helical domain, an intermolecular interaction domain, and a heterologous component linked to the 5′ or 3′ end of the pRNA.

6. The pRNA chimera of claim 5, wherein the heterologous component comprises a targeting moiety.

7. The pRNA chimera of claim 6, wherein the targeting moiety comprises folate.

8. The pRNA chimera of claim 7, wherein the folate is linked to a 5′ overhanging end of the pRNA.

9. The pRNA chimera of claim 5, wherein the heterologous component comprises a detectable label.

10. A pRNA chimera comprising a paired double-stranded helical domain, an intermolecular interaction domain, and an oligonucleotide annealed to the 5′ or 3′ end of the pRNA.

11. The pRNA chimera of claim 10 wherein the oligonucletoide comprises a DNA oligonucleotide.

12. The pRNA chimera of claim 10 wherein the oligonucleotide comprises a detectable label.

13. The pRNA chimera of claim 12 wherein the detectable label comprises biotin or a radiolabel.

14. The pRNA chimera of any of claim 10 wherein the pRNA comprises a 3′ overhanging end, and wherein the oligonucleotide is annealed to the 3′ overhanging end.

15. A polyvalent multimeric pRNA complex comprising a plurality of pRNA chimeras, said pRNA chimeras each independently comprising a paired double-stranded helical domain and an intermolecular interaction domain, wherein at least one pRNA chimera is selected from the group consisting of:
(a) a pRNA chimera wherein the paired double-stranded helical domain comprises a heterologous component comprising an siRNA;
(b) a pRNA chimera further comprising a heterologous component linked to the 5′ or 3′ end of the pRNA; and
(c) a pRNA chimera further comprising an oligonucleotide annealed to the 5′ or 3′ end of the pRNA.

16. The polyvalent multimeric pRNA complex of claim 15 wherein at least one PRNA chimera comprises a targeting moiety.

17. The polyvalent multimeric pRNA complex of claim 16 wherein the targeting moiety comprises an RNA aptamer.

18. The polyvalent multimeric pRNA complex of claim 16 wherein the targeting moiety comprises an antibody.

19. The polyvalent multimeric pRNA complex of claim 16 wherein the targeting moiety comprises a receptor ligand.

20. The polyvalent multimeric pRNA complex of claim 19 wherein the receptor ligand comprises folate.

21. The polyvalent multimeric pRNA complex claim 15 wherein least one pRNA chimera is a circularly permuted pRNA chimera.

22. The polyvalent multimeric pRNA complex of claim 16 wherein the targeting moiety is conjugated to the 5′ or 3′ end of a non-circularly permuted pRNA.

23. The polyvalent multimeric pRNA complex of claim 15 wherein at least one pRNA chimera comprises a heterologous component comprising a therapeutic agent.

24. The polyvalent multimeric pRNA complex of claim 15 wherein at least one pRNA chimera comprises a heterologous component comprising an endosome disrupting agent.

25. The polyvalent multimeric pRNA complex of claim 15 wherein at least one pRNA chimera comprises at least one nonnative polynucleotide or polynucleotide bond.

26. The polyvalent multimeric pRNA complex of claim 25 wherein the nonnative polynucleotide comprises a nucleotide selected from the group consising of a 2′-NH₂-2′-deoxy CTP, 2′-CH₃-2′-deoxy CTP, 2′-F-2′ deoxy CTP, 2′-F-2′ deoxy UTP, and a spiegelmer.

27. The polyvalent multimeric pRNA complex of claim 15 comprising at least one circularly permuted pRNA chimera and at least one non-circularly permuted pRNA chimera.

28. The polyvalent multimeric pRNA complex of claim 15 which is a dimer, a trimer or a hexamer.

29. The polyvalent multimeric pRNA complex of claim 15 wherein at least one pRNA chimera comprises a biologically active moiety selected from the group consisting of a ribozyme, a siRNA, an RNA aptamer, an antisense RNA and a peptide nucleic acid (PNA).

30. A pRNA chimera comprising a paired double-stranded helical domain and an intermolecular interaction domain, the pRNA chimera further comprising at one nonnative polynucleotide or polynucleotide bond.

31. The pRNA chimera of claim 30 wherein the nonnative polynucleotide comprises a nucleotide selected from the group consisting of a 2′-NH₂-2′-deoxy CTP, 2′-CH₃-2′-deoxy CTP, 2′-F-2′ deoxy CTP, 2′-F-2′ deoxy UTP, and a spiegelmer.

32. A method for delivering a therapeutic agent to a host cell comprising:
contacting the cell with the polyvalent multimeric pRNA complex of claim 15, wherein said polyvalent multimeric pRNA complex comprises a first pRNA chimera comprising therapeutic agent and a second pRNA chimera comprising a targeting moiety, such that the polyvalent multimeric complex is taken up by the host cell.

33. The method of claim 32 wherein the targeting moiety binds to a receptor, and wherein the polyvalent multimeric complex is taken up by the cell via receptor-mediated endocytosis.

34. The method of claim 32 wherein the targeting moiety comprises folate.

35. The method of claim 32 wherein the therapeutic agent comprises an siRNA, a ribozme or an antisense RNA.

36. The method of claim 32 wherein the cell is present in a cell, a cell culture, a tissue, an organ or an organism.

37. The method of claim 32 wherein the cell is a mammalian cell.

38. The method of claim 36 wherein the cell is a human cell.


SEQUENCE LISTING (78 sequences)

SEQ ID NO: 1 - RNA, 120 nt, Bacteriophage phi-29
ucaaugguacgguacuuccauugucauguguauguuggggauuaaacccugauugaguucagcccacauacuuuguugauugguugucaaucauggcaaaagugcacgcuacuuugauaa

SEQ ID NO: 2 - DNA, 15 bp, Artificial Sequence
  Description of Artificial Sequence Synthetic DNA template
ttcctaaaggaatgg

SEQ ID NO: 3 - RNA, 168 nt, Artificial Sequence
  Description of Artificial Sequence Synthetic pRNA chimera
guugauugguugucaaucauggcaaaagugcacgcuacuuugaaaaacaaauucuaaaacugaugaguccgugaggacgaaagcuguaacacaaaaucaaugguacgguacuuccauugucauguguauguuggggauuaaacccugauugaguucagcccacauaca

SEQ ID NO: 4 - RNA, 24 nt, Artificial Sequence
  Description of Artificial Sequence Synthetic portion of U7 snRNA substrate
guguuacagcucuuuuagaauuug

SEQ ID NO: 5 - RNA, 46 nt, Artificial Sequence
  Description of Artificial Sequence Synthetic anti-12-LOX ribozyme
acgcgguuguaccugaugaguccgugaggacgaaacccggagaaga

SEQ ID NO: 6 - RNA, 25 nt, Artificial Sequence
  Description of Artificial Sequence Synthetic RNAsubstrate
ucuucuccgggucguacaaccgcgu

SEQ ID NO: 7 - RNA, 55 nt, Artificial Sequence
  modified_base (25)..(32): any nucleotide and this region may be 4-8 nucleotides, preferably 4 or 5 nucleotides
uugucauguguauguuggggauuannnnnnnncugauugaguucagcccacauac

SEQ ID NO: 8 - DNA, 17 bp, Artificial Sequence
  Description of Artificial Sequence Synthetic DNA loop
taatacgactcactata

SEQ ID NO: 9 - RNA, 94 nt, Artificial Sequence
  Description of Artificial Sequence Synthetic U7 template
gggaaagcuuauaguguuacagcucuuuuagaauuugucuagcagguuuucugacuucggucggaaaacgccuaacguugcaugccugcagguc

SEQ ID NO: 10 - RNA, 47 nt, Artificial Sequence
  Description of Artificial Sequence Synthetic RzU7 template
ggcaaauucuaaaacugaugaguccgugaggacgaaagcuguaacac

SEQ ID NO: 11 - RNA, 68 nt, Artificial Sequence
  Description of Artificial Sequence Synthetic permuted SF5 pRNA chimera
gugagacaucguauuuacuauaauguaugugugucggguuguuuucggauugaguuccgccgacagca

SEQ ID NO: 12 - RNA, 72 nt, Artificial Sequence
  Description of Artificial Sequence Synthetic permuted B103 pRNA chimera
gacaaggugcaacacuuccuauaguauggugcgugauugggguauauccugauugaguucagcccacacgcc

SEQ ID NO: 13 - RNA, 71 nt, Artificial Sequence
  Description of Artificial Sequence Synthetic permuted phi29/PZA pRNA chimera
ucaaugguacgguacuuccauugucauguguauguuggggauuaaacccugauugaguucagcccacauac

SEQ ID NO: 14 - RNA, 72 nt, Artificial Sequence
  Description of Artificial Sequence Synthetic permuted M2/NF pRNA chimera
gacaaggugcaacacuuccuauaguauggcacaugauugggguauauccugauugaguucagcccacauguc

SEQ ID NO: 15 - RNA, 68 nt, Artificial Sequence
  Description of Artificial Sequence Synthetic permuted GA1 pRNA chimera
uaaggcauugagguuaaaauauauaggcugugcaacgggcauccuacuguaaaaaguaccguugacag

SEQ ID NO: 16 - RNA, 75 nt, Artificial Sequence
  Description of Artificial Sequence Synthetic permuted aptamer pRNA
ggaaugguacgguacuuccauugucauguguaggguugggaagaaacuguggcacuucggugccagcaacccuac

SEQ ID NO: 17 - RNA, 188 nt, Artificial Sequence
  Description of Artificial Sequence Synthetic ribozyme pRNA-RzA transcript
gcuaguucuagaguugauugguugucaaucauggcaaaagugcacgcuacuuugcaaaacaaauucuuuacugaugaguccgugaggacgaaacgggucaaaagcaaugguacgguacuuccauugucauguguauguuggggauuaaacccugauugaguucagcccacauacgguaccucgacguc

SEQ ID NO: 18 - RNA, 66 nt, Artificial Sequence
  Description of Artificial Sequence Synthetic ribozyme RzA transcript
gcuaguucuagacaaauucuuuacugaugaguccgugaggacgaaacgggucgguaccucgacguc

SEQ ID NO: 19 - RNA, 10 nt, Artificial Sequence
  Description of Artificial Sequence Synthetic 3′ overhang oligonucleotide
aagccgaauu

SEQ ID NO: 20 - RNA, 59 nt, Artificial Sequence
  Description of Artificial Sequence Synthetic 3′ overhang oligonucleotide
aagccgaauuccagcacacuggcggccguuacuaguggauccgagcucgguaccaagcu

SEQ ID NO: 21 - RNA, 54 nt, Artificial Sequence
  Description of Artificial Sequence Synthetic 3′ overhang oligonucleotide
ccuuuuacaugcgacacagacgaagcgcuaaaacgugggauucugugucguuuu

SEQ ID NO: 22 - RNA, 120 nt, Artificial Sequence; identical to SEQ ID NO: 1
  Description of Artificial Sequence Synthetic 3′ overhang oligonucleotide

SEQ ID NO: 23 - RNA, 40 nt, Artificial Sequence
  Description of Artificial Sequence Synthetic hammerhead ribozyme
caaauucuuuacugaugaguccgugaggacgaaacggguc

SEQ ID NO: 24 - RNA, 19 nt, Artificial Sequence
  Description of Artificial Sequence Synthetic HBV polyAtarget
gacccguauaaagaauuug

SEQ ID NO: 25 - RNA, 162 nt, Artificial Sequence
  Description of Artificial Sequence Synthetic ribozyme pRNA-RZA
guugauugguugucaaucauggcaaaagugcacgcuacuuugaaaaacaaauucuuuacugaugaguccgugaggacgaaacgggucaaaaucaaugguacgguacuuccauugucauguguauguuggggauuaaacccugauugaguucagcccacauac

SEQ ID NO: 26 - RNA, 119 nt, Bacteriophage phi-29
gcaaugguacgguacuuccauugucauguguauguuggggauuaacgccugauugaguucagcccacauacuuuguugauuguccgucaaucauggcaaaagugccgcuacuuugcuaa

SEQ ID NO: 27 - RNA, 117 nt, Bacteriophage phi-29
ucaaugguacgguacuuccauugucauguguauguuggggauuaaacccugauugaguucagcccacauacuuuguugauugguugucaaucauggcaaaagugcacgcuacuuuga

SEQ ID NO: 28 - RNA, 44 nt, Artificial Sequence
  Description of Artificial Sequence Synthetic permuted SF5 pRNA chimera
gggagcguaaaacacucuugcauuaaaaauaugacugauuucgc

SEQ ID NO: 29 - RNA, 43 nt, Artificial Sequence
  Description of Artificial Sequence Synthetic permuted B103 pRNA chimera
ggggauugauagcccucuuacuaaaagugauuguuucuuuguc

SEQ ID NO: 30 - RNA, 43 nt, Artificial Sequence
  Description of Artificial Sequence Synthetic permuted phi29/PZA pRNA chimera
guugauugguugucaaucauggcaaaagugcacgcuacuuuga

SEQ ID NO: 31 - RNA, 43 nt, Artificial Sequence
  Description of Artificial Sequence Synthetic permuted M2/NF pRNA chimera
ggggauugauaacccucuuacuaaaagugauuguuucuuuguc

SEQ ID NO: 32 - RNA, 42 nt, Artificial Sequence
  Description of Artificial Sequence Synthetic permuted GA1 pRNA chimera
uguugcgugguagcaauacuauauauacucucucucguuuua

SEQ ID NO: 33 - RNA, 43 nt, Artificial Sequence
  Description of Artificial Sequence Synthetic permuted aptamer pRNA
guugauugguugucaaucauggcaaaagugcacgcuacuuucc

SEQ ID NO: 34 - RNA, 71 nt, Artificial Sequence
  Description of Artificial Sequence Synthetic circularly permuted pRNA
gcaaugguacgguacuuccauugucauguguauguuggggauuaaacccugauugaguucagcccacauac

SEQ ID NO: 35 - RNA, 43 nt, Artificial Sequence
  Description of Artificial Sequence Synthetic circularly permuted pRNA
guugauugguugucaaucauggcaaaagugcacgcuacuuugc

SEQ ID NO: 36 - RNA, 157 nt, Artificial Sequence
  modified_base (29)..(31): a, c, t, g, unknown or other
guacuguuaccuucauggcaugguaacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncguuucaucgcacgugaaaacgg

SEQ ID NO: 37 - RNA, 119 nt, Artificial Sequence
  Description of Artificial Sequence Synthetic cpRNA transcript
ggaaugguacgguacuccauugucauguguauguuggggauuaaacccugauugaguucagcccacauacuuuguugauugguugucaaucauggcaaagugcacgcuacuuuccuaaa

SEQ ID NO: 38 - DNA, 29 bp, Artificial Sequence
  Description of Artificial Sequence Synthetic DNA template
ttccttaatacgactcactataggaatgg

SEQ ID NO: 39 - RNA, 133 nt, Artificial Sequence
  Description of Artificial Sequence Synthetic cpRNA transcript
ggaaugguacgguacuccauugucauguguauguuggggauuaaacccugauugaguucagcccacauacuuuguugauugguugucaaucauggcaaagugcacgcuacuuuccuuaauacgacucacuaua

SEQ ID NO: 40 - RNA, 120 nt, Artificial Sequence
  Description of Artificial Sequence Synthetic phi29 viral particle
ucaaugguacgguacuuccauugucauguguauguuggggauuaggaccugauugaguucagcccacauacuuuguugauugcgugucaaucauggcaaaagugcacgcuacuuugauaa

SEQ ID NO: 41 - RNA, 120 nt, Artificial Sequence
  Description of Artificial Sequence Synthetic chimeric pRNA
guugauugcgugucaaucauggcuuuugagaagcuucugaugaguccgugaggacgaaaugagucuguuuugucauguguauguuggggauuaggaccugauugaguucagcccacauac

SEQ ID NO: 42 - RNA, 108 nt, Artificial Sequence
  Description of Artificial Sequence Synthetic chimeric pRNA
guugauuugucgucaaucauggccucagagacagagcagaaacgacaguucaagccgaagucauguguauguuggggauuaacgccugauugaguucagcccacauac

SEQ ID NO: 43 - RNA, 72 nt, Artificial Sequence
  Description of Artificial Sequence Synthetic chimeric pRNA
gucauguguauguuggggauuagacacugauugaguucagcccacauacguugauuguccgucaaucauggc

SEQ ID NO: 44 - RNA, 19 nt, Artificial Sequence
  Description of Artificial Sequence Synthetic chimeric pRNA
cagacucaucaagcuucuc

SEQ ID NO: 45 - RNA, 28 nt, Artificial Sequence
  modified_base (7)..(14): any nucleotide and this region may be 4-8 nucleotides, preferablly 4 or 5 nucleotides
guugaunnnnnnnngucaaucauggcaa

SEQ ID NO: 46 - RNA, 126 nt, Artificial Sequence
  Description of Artificial Sequence Syntheticoligonucleotide
aggaggguguuugcaaaugauuuuugucauguguauguuggggauuagccacugauugaguucagcccacauacuuuguugauuguccgucaaucauggcaauuuucauuugcuuucacccuccuu

SEQ ID NO: 47 - RNA, 108 nt, Artificial Sequence
  Description of Artificial Sequence Synthetic oligonucleotide
guugauugcgugucaaucauggccucagagacagagcagaaacgacaguucaagccgaagucauguguauguuggggauuaggaccugauugaguucagcccacauac

SEQ ID NO: 48 - RNA, 117 nt, Artificial Sequence
  Description of Artificial Sequence Synthetic oligonucleotide
ucaaugguacgguacuuccauugucauguguauguuggggauuaacgccugauugaguucagcccacauacuuuguugauuuggcgucaaucauggcaaaagugcacgcuacuuuga

SEQ ID NO: 49 - RNA, 123 nt, Artificial Sequence
  Description of Artificial Sequence Synthetic oligonucleotide
agaacggcaucaaggugaacuucaagauuuuguauguuggggauuaggaccugauugaguucagcccacauacuuuguugauugcgugucaauuuaucuugaaguucaccuugaugccguucu

SEQ ID NO: 50 - RNA, 117 nt, Bacteriophage phi-29
agaaugguacgguacuuccauugucauguguauguuggggauuaggaccugauugaguucagcccacauacuuuguugauugcgugucaaucauggcaaaagugcacgcuacuuucu

SEQ ID NO: 51 - RNA, 117 nt, Bacteriophage phi-29
ucaaugguacgguacuuccauugucauguguauguuggggauuaggaccugauugaguucagcccacauacuuuguugauugcgugucaaucauggcaaaagugcacgcuacuuuga

SEQ ID NO: 52 - RNA, 111 nt, Artificial Sequence
  Description of Artificial Sequence Synthetic oligonucleotide
guugauuguccgucaaucuaggccucagagacagagcagaaacgacaguucaagccgaagucauguguauguuggggauuaacgccugauugaguucagcccacauacuuu

SEQ ID NO: 53 - RNA, 108 nt, Artificial Sequence
  Description of Artificial Sequence Synthetic oligonucleotide
ggaccaccgcaucucuacauuuuguauguuggggauuggaccugauugaguucagcccacauacuuuguugauugcgugucaauuuauguagacaugcggugguccuu

SEQ ID NO: 54 - RNA, 82 nt, Artificial Sequence
  Description of Artificial Sequence Synthetic oligonucleotide
agguacgcgguacuaacugccuguuaguuguuucauacacccgacuugaguuaguccgcaauuagggguuguauguguacug

SEQ ID NO: 55 - RNA, 121 nt, Artificial Sequence
  Description of Artificial Sequence Synthetic oligonucleotide
ccuaagguugagucgcucucggugggcuauuuaacugugcguuaguuguuucauacacccgacuugaguuaguccagggggguuguauguuuuaguccaccgagggcgacuuaaccuuagg

SEQ ID NO: 56 - RNA, 117 nt, Bacteriophage phi-29
ucaaugguacgguacuuccauugucauguguauguuggggauuaggaccugauugaguucagcccacauacuuuguugauuguccgucaaucauggcaaaagugcacgcuacuuuga

SEQ ID NO: 57 - RNA, 100 nt, Artificial Sequence
  Description of Artificial Sequence Synthetic oligonucleotide
guacgguacuuccauugucauguguauguuggggauuaggaccugauugaguucagcccacauacuuuguugauugcgugucaaucauggcaaaagugca

SEQ ID NO: 58 - RNA, 100 nt, Artificial Sequence
  Description of Artificial Sequence Synthetic oligonucleotide
guacgguacuuccauugucauguguauguuggggauuaacgccugauugaguucagcccacauacuuuguugauuguccgucaaucauggcaaaagugca

SEQ ID NO: 59 - RNA, 108 nt, Artificial Sequence
  Description of Artificial Sequence Synthetic oligonucleotide
ggaccaccgcaucucuacauuuuguauguuggggauuggagcugauugaguucagcccacauacuuuguugauugcgugucaauuuauguagagaugcggugguccuu

SEQ ID NO: 60 - RNA, 22 nt, Artificial Sequence
  Description of Artificial Sequence Synthetic oligonucleotide
ccgcaucucuacauucaagaac

SEQ ID NO: 61 - RNA, 159 nt, Artificial Sequence
  Description of Artificial Sequence Synthetic oligonucleotide
guugauugcgugucaaucauggcaaaagugcacgcuacuuuccaaaacuugaaugcugaugaggccgaaaggccgaaagagaugaaaaggaaugguacgguacuuccauugucauguguauguuggggauuaggaccugauugaguucagcccacauac

SEQ ID NO: 62 - RNA, 19 nt, Artificial Sequence
  Description of Artificial Sequence Synthetic oligonucleotide
gacccguauaaagaauuug

SEQ ID NO: 63 - RNA, 163 nt, Artificial Sequence
  Description of Artificial Sequence Synthetic oligonucleotide
guuugauugcgugucaaucauggcaaaagugcacgcuacuuugaaaaacaaauucuuuacugaugaguccgugaggacgaaacgggucaaaaucaaugguacgguacuuccauugucauguguauguuggggauuaggaccugauugaguucagcccacauac

SEQ ID NO: 64 - RNA, 159 nt, Artificial Sequence
  Description of Artificial Sequence Synthetic oligonucleotide
guugauuguccgucaaucauggcaaaagugcacgcuacuuugaaaaacuugaaugcugaugaggccgaaaggccgaaagagaugaaaaucaaugguacgguacuuccauugucauguguauguuggggauuaacgccugauugaguucagcccacauac

SEQ ID NO: 65 - RNA, 100 nt, Artificial Sequence
  Description of Artificial Sequence Synthetic oligonucleotide
guacgguacuuccauugucauguguauguuggggauuaacgccugauugaguucagcccacauacuuuguugauuuggcgucaaucauggcaaaagugca

SEQ ID NO: 66 - RNA, 117 nt, Artificial Sequence
  Description of Artificial Sequence Synthetic oligonucleotide
ucaaugguacgguacuuccauugucauguguauguuggggauuagccacugauugaguucagcccacauacuuuguugauuguccgucaaucauggcaaaagugcacgcuacuuuga

SEQ ID NO: 67 - RNA, 159 nt, Artificial Sequence
  Description of Artificial Sequence Synthetic oligonucleotide
guugauugcgugucaaucauggcaaaagugcacgcuacuuugaaaaacuugaaugcugaugaggccgaaaggccgaaagagaugaaaaucaaugguacgguacuuccauugucauguguauguuggggauuaggaccugauugaguucagcccacauac

SEQ ID NO: 68 - RNA, 101 nt, Artificial Sequence
  Description of Artificial Sequence Synthetic oligonucleotide
aguacgguacuuccauugucauguguauguuggggauuaacgccugauugaguucagcccacauacuuuguugauuuggcgucaaucauggcaaaagugca

SEQ ID NO: 69 - DNA, 73 bp, Artificial Sequence
  Description of Artificial Sequence Synthetic oligonucleotide
gcacgctactttccaaaacttgaatgctgatgaggccgaaaggccgaaagagatgaaaaggaatggtacggta

SEQ ID NO: 70 - DNA, 72 bp, Artificial Sequence
  Description of Artificial Sequence Synthetic oligonucleotide
gcacgctactttccaaaacttgaatgctgatgaggccgaaaggccaaagagatgaaaaggaatggtacggta

SEQ ID NO: 71 - DNA, 72 bp, Artificial Sequence
  Description of Artificial Sequence Synthetic oligonucleotide
gcacgctactttccaaaacttgaatgctaatgaggccgaaaggccaaagagatgaaaaggaatggtacggta

SEQ ID NO: 72 - DNA, 60 bp, Artificial Sequence
  Description of Artificial Sequence Synthetic oligonucleotide
taatacgactcactatagttgattgcgtgtcaatcatggcaaaagtgcacgctactttcc

SEQ ID NO: 73 - DNA, 71 bp, Artificial Sequence
  Description of Artificial Sequence Synthetic oligonucleotide
gtatgtgggctgaactcaatcaggtcctaatccccaacatacacatgacaatggaagtaccgtaccattcc

SEQ ID NO: 74 - RNA, 143 nt, Artificial Sequence
  Description of Artificial Sequence Synthetic oligonucleotide
agaaugguacgguacuuccauugucauguguauguuggggauuaaacccugauugaguucagcccacauacuuuguugauugguugucaaucauggcaaaagugcacgcuacuuucuaaucccgcggccauggcggccgggag

SEQ ID NO: 75 - DNA, 26 bp, Artificial Sequence
  Description of Artificial Sequence Synthetic oligonucleotide
ctcccggccgccatggccgcgggatt

SEQ ID NO: 76 - DNA, 22 bp, Artificial Sequence
  Description of Artificial Sequence Synthetic primer
aaagagccaagaacaaaattgc

SEQ ID NO: 77 - DNA, 23 bp, Artificial Sequence
  Description of Artificial Sequence Synthetic primer
gagagagaagcagccactgttac

SEQ ID NO: 78 - RNA, 26 nt, Artificial Sequence
  Description of Artificial Sequence Synthetic oligonucleotide
aaucccgcggccauggcggccgggag